(19) United States
(12) Patent Application Publication
(10) Pub. No.: US 2018/0235931 A1
(43) Pub. Date: Aug. 23, 2018

(54) USE OF NEUROKININ-1 ANTAGONISTS AS ANTITUSSIVES

(71) Applicant: Menlo Therapeutics Inc., Redwood City, CA (US)

(72) Inventors: Steven BASTA, Redwood City, CA (US); Paul KWON, Redwood City, CA (US); Xiaoming ZHANG, Redwood City, CA (US); Cyrus K. BECKER, Redwood City, CA (US); Meenakshi S. VENKATRAMAN, Redwood City, CA (US)

(21) Appl. No.: 15/873,732

(22) Filed: Jan. 17, 2018

Related U.S. Application Data
(60) Provisional application No. 62/447,105, filed on Jan. 17, 2017.

(51) Int. Cl. A61K 31/403; A61K 45/06; A61P 11/14; A61K 9/20

(52) U.S. Cl. CPC A61K 31/403; A61K 9/20; A61P 11/14; A61K 45/06

(57) ABSTRACT

The disclosure relates to the use of neurokinin-1 (NK-1) antagonists, such as serlopitant, MK-0303 or MK-8478, in alleviating or suppressing cough (including acute, subacute and chronic cough) and urge to cough. The cough can have an unknown cause (idiopathic cough) or can be associated with any type of medical condition, such as a respiratory disorder. The NK-1 antagonist can be delivered by oral inhalation for more rapid peripheral and central antitussive action. Another antitussive agent in addition to the NK-1 antagonist can optionally be administered for the treatment of the cough or urge to cough, or the cough-associated medical condition.

### RELATED APPLICATIONS

This application claims priority to and the benefit of U.S. Provisional Patent Application No. 62/447,105 filed on Jan. 17, 2017, which is incorporated herein by reference in its entirety for all purposes.

### TECHNICAL FIELD

The present disclosure relates to the use of a neurokinin-1 (NK-1) antagonist, such as serlopitant, MK-0303 or MK-8478, to alleviate or suppress cough (including acute, subacute and chronic cough) and urge to cough.

### BACKGROUND OF THE DISCLOSURE

A cough is a sudden and often repetitively occurring reflex that helps to clear the airways, including the lungs, of fluids, irritants, foreign particles and microbes. Frequent coughing usually indicates the presence of a disease. Irregular coughing is often caused by a respiratory tract infection. Cough is the most common reason for visits to a primary care physician in the United States. About 5-10% of adults suffer from chronic cough, and about two-thirds of chronic cough sufferers are women. Chronic cough, which may not have an obvious underlying cause and may last for years, can be distressing and functionally disabling.

The respiratory tract, or airways, participates in the vital process of gas exchange in order to support the demand for oxygen intake and carbon dioxide elimination. Vagal autonomic nerves control smooth muscles of the tracheobronchial tree, and thus caliber of airways, as well as liberation and movement of secretions (e.g., mucus and fluid). Control is coordinated within brainstem nuclei that regulate voluntary and autonomic outflow, relying on a rich input of vagal sensory signals from the airway tissues that in turn convey sensations and trigger autonomic reflexes. Vagal sensory fibers arise mostly from cell bodies within jugular and nodose ganglia, and their activity is regulated by a range of chemical substances.

### SUMMARY OF THE DISCLOSURE

The present disclosure provides for the use of an antagonist (or inhibitor) of neurokinin-1 (NK-1) in relieving or suppressing cough (including acute, subacute and chronic cough) and urge to cough. The NK-1 antagonist reduces symptoms and complications of the cough condition, such as the frequency, the severity and the impact thereof. The cough can have a known cause or an unknown cause (idiopathic cough), and can be associated with any type of medical condition, such as a respiratory disorder or gastroesophageal reflux disease. In some embodiments, the NK-1 antagonist is used to treat chronic cough. In certain embodiments, the NK-1 antagonist is serlopitant, MK-0303 or MK-8478, or a pharmaceutically acceptable salt, solvate, hydrate, clathrate, polymorph, prodrug or metabolite thereof. In some embodiments, the NK-1 antagonist is administered by oral inhalation for more rapid antitussive action peripherally in the airways and centrally in the brainstem. Another antitussive agent in addition to the NK-1 antagonist can optionally be administered for the treatment of the cough or urge to cough, or the cough-associated medical condition.

### BRIEF DESCRIPTION OF THE DRAWINGS

A better understanding of features and advantages of the present disclosure will be obtained by reference to the following detailed description, which sets forth illustrative embodiments of the disclosure, and the accompanying drawings.

FIG. 1 illustrates a Franz diffusion cell for studying skin permeation of a drug in vitro.

FIG. 2 shows the cumulative release of serlopitant from topical formulations B and C into the receptor chamber of a Franz diffusion cell at various time points in an in vitro study of skin permeation.

FIG. 3 shows the amount of serlopitant (called “VPD737”) retained in the skin at the end of the Franz diffusion cell study. Each bar represents ug of serlopitant/g of skin in 250 um skin layers. For each of topical formulations B and C, the bars from left to right represent the amount of serlopitant retained in skin layers from the stratum corneum to the dermis.

### DETAILED DESCRIPTION OF THE DISCLOSURE

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein can be employed in practicing the disclosure. It is also understood that every embodiment of the disclosure can optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act, the order of the acts of the method is not necessarily limited to the order in which the acts of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

It is further understood that the present disclosure encompasses analogs, derivatives, prodrugs, metabolites, salts, solvates, hydrates, clathrates and polymorphs of all of the compounds/substances disclosed herein, as appropriate. The specific recitation of “analogs”, “derivatives”, “prodrugs”, “metabolites”, “salts”, “solvates”, “hydrates”, “clathrates” or “polymorphs” with respect to a compound/substance or a group of compounds/substances in certain instances of the disclosure shall not be interpreted as an intended omission of any of these forms in other instances of the disclosure where the compound/substance or the group of compounds/substances is mentioned without recitation of any of these forms, unless stated otherwise or the context clearly indicates otherwise.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

### Definitions

Unless defined otherwise or clearly indicated otherwise by their use herein, all technical and scientific terms used herein have the same meaning as commonly understood by one of ordinary skill in the art to which this application belongs.

As used in the specification and the appended claims, the indefinite articles “a” and “an” and the definite article “the” can include plural referents as well as singular referents unless specifically stated otherwise or the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within ±20%, 15%, 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

The abbreviation “aka” denotes “also known as”.

The term “antagonists” includes neutral antagonists and inverse agonists.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” carrier or excipient of a pharmaceutical composition is also compatible with the other ingredients of the composition.

The term “therapeutically effective amount” refers to an amount of a substance that, when administered to a subject, is sufficient to prevent, reduce the risk of developing, delay the onset of, slow the progression of, or cause regression of the medical condition being treated, or to alleviate to some extent the medical condition or one or more symptoms or complications of that condition. The term “therapeutically effective amount” also refers to an amount of a substance that is sufficient to elicit the biological or medical response of a cell, tissue, organ, system, animal or human which is sought by a researcher, veterinarian, medical doctor or clinician.

The terms “treat”, “treating” and “treatment” include alleviating, ameliorating or abrogating a medical condition or one or more symptoms or complications associated with the condition, and alleviating, ameliorating or eradicating one or more causes of the condition. Reference to “treatment” of a medical condition includes preventing (precluding), reducing the risk of developing, delaying the onset of, slowing the progression of, and causing regression of the condition or one or more symptoms or complications associated with the condition.

The term “medical conditions” (or “conditions” for short) includes diseases and disorders.

The term “subject” refers to an animal, including a mammal, such as a primate (e.g., a human, a chimpanzee or a monkey), a rodent (e.g., a rat, a mouse, a gerbil, a hamster or a guinea pig), a lagomorph (e.g., a rabbit), a swine (e.g., a pig), an equine (e.g., a horse), a canine (e.g., a dog) or a feline (e.g., a cat). The terms “subject” and “patient” are used interchangeably herein in reference, e.g., to a mammalian subject, such as a human subject.

Treatment of Cough with a Neurokinin-1 Antagonist

Coughing is an airway defensive/protective reflex for removing irritants and foreign materials from the airways. For example, the cough reflex results in the removal of foreign material from the bronchi, with successive coughs forcing the foreign material from the smaller bronchi to the larger and mainstem bronchi and toward the trachea so that the foreign material can be expelled. Coughing is most easily evoked by irritant stimulation of the larynx, trachea and larger bronchi.

Coughing is due to activation of receptors on sensory nerves in the upper and lower respiratory tracts which sends impulses via vagal afferent pathways to the brainstem respiratory center. The vagal afferent pathways associated with coughing involve receptors on endings of sensory afferent fibers terminating peripherally in and close below the epithelium of the airways. Different subtypes of myelinated (faster conducting) vagal sensory Aδ-fibers express cough receptors, rapidly adapting stretch receptors (RARs) and slowly adapting stretch receptors (SARs) that can initiate coughing upon direct stimulation by tussigenic agents (however, these receptors are insensitive to capsaicin and anesthesia) or/and can enhance and facilitate coughing. Unlike RARs and SARs, cough receptors do not respond to changes in lung volume, but rather are very sensitive to punctuate (touch-like) mechanical stimuli, rapid reduction in airway pH and hypotonic solutions. According to Mazzone and Canning, “cough receptors are ideally situated and responsive to initiate cough following aspiration, inhalation of particulate matter or in response to accumulated secretions”, which suggests that it would desirable to activate cough receptors in order to promote productive cough. Cough receptor Aδ-fibers terminate primarily in the mucosa between the epithelium and the smooth muscle of the extrapulmonary airways (including the larynx, trachea and mainstem bronchi), while RAR and SAR Aδ-fibers innervate primarily the intrapulmonary airways (including the lungs). See, e.g., LaVinka 2013, Canning, RPN 2006 and Mazzone 2009.

In addition to cough receptors, RARs and SARs, the cough reflex can be initiated by stimulation of another class of vagal afferent nerves innervating the airways—unmyelinated (slower conducting) C-fibers. Cough-evoking unmyelinated C-fibers have extensive endings superficially in and close below the epithelium of the airways (including the trachea, bronchi and lungs), whose activation by cough stimulants such as capsaicin and bradykinin evokes cough when the subject is awake but not when anesthetized. The unmyelinated C-fibers detect a wide range of potentially noxious stimuli, including exogenous chemicals and endogenous inflammatory molecules, and become activated in response to tissue irritation or inflammation. Moreover, activation of unmyelinated C-fibers (e.g., centrally in the nucleus tractus solitarius [NTS] in the dorsomedial medulla) can increase cough reflex sensitivity by sensitizing the pathways receiving input from cough receptors or RARs in the airways (e.g., by reducing the threshold for initiating the cough reflex via stimulation of cough receptors or RARs). The C-fibers initiate cough to remove an irritating or “itchy” feeling in the throat, which is more characteristic of unproductive (dry) cough and more typical of chronic cough. In airway diseases such as chronic obstructive pulmonary disease (COPD) and asthma and in extrapulmonary diseases such as allergic rhinitis and gastroesophageal reflux disease (GERD), activation of unmyelinated C-fibers by inflammatory mediators or acid can sensitize the cough reflex via central interaction (e.g., in the NTS) of the C-fibers with cough receptor and RAR myelinated Aδ-fibers. Such afferent nerve interaction and the resulting sensitization can increase coughing responses to tussigenic stimuli, so peripheral and central inhibition of C-fiber activation can suppress, e.g., unproductive (dry) cough and chronic cough. See, e.g., LaVinka 2013, Keller 2017 and Mazzone 2005.

The cough receptor-, RAR- and SAR-expressing vagal myelinated Aδ-fibers and the vagal unmyelinated C-fibers interact with each other, including centrally in the brainstem, in response to irritating stimuli to modulate the cough reflex. The cell bodies of the myelinated Aδ-fibers are located primarily in the nodose ganglion (the inferior ganglion of vagus nerve), and those of cough-evoking unmyelinated C-fibers are located primarily in the jugular ganglion (the superior ganglion of vagus nerve). Vagal afferent nerves are the primary communication pathways between the bronchopulmonary system and the central nervous system (CNS). Signals from the airway vagal sensory fibers are transmitted to brainstem sensory nuclei for initial processing. The brainstem nuclei then send signals to the brainstem respiratory central pattern generator to produce the cough motor pattern for reflexive coughing, as well as to higher brain regions for the perception of airway irritation and for behavioral modulation of coughing. See, e.g., Canning, RPN 2006, LaVinka 2013 and Keller 2017.

Tachykinin neuropeptides are involved in increased cough reflex sensitivity.

Tachykinins, including substance P (the most potent tachykinin) and neurokinins A and B, are present in the sensory nerve fibers in the upper and lower airway tracts (e.g., vagal airway unmyelinated C-fibers produce and release tachykinins, including substance P and neurokinins A and B), although cough receptor vagal afferent myelinated Aδ-fibers do not produce tachykinins. Stimulation of sensory nerves in the airways, especially in response to irritant stimuli, induces release of tachykinins, including substance P and neurokinins A and B, from these afferent nerves, and their release sensitizes the cough reflex. For instance, activation of TRPA1 or TRPV1 on vagal sensory unmyelinated C-fibers innervating the airways leads to release of inflammatory neuropeptides from the C-fibers, including the tachykinins substance P and neurokinins A and B. Furthermore, substance P stimulates bronchopulmonary RAR activity—e.g., substance P causes plasma extravasation in the airways, which can activate RARs (Bonham 1996). Moreover, degranulated mast cells release inflammatory mediators that activate unmyelinated C-fibers, which then secrete substance P. Secretion of substance P results in bronchoconstriction, vasodilation, inflammation and sensitization of nerves to the cough reflex. In the airways, tachykinins, including substance P and neurokinin A, are released from airway sensory nerves and evoke bronchoconstriction, vasodilation, microvascular leakage, plasma protein extravasation, mucus secretion, leukocyte recruitment, inflammation (neurogenic inflammation) and airway hyperreactivity, which individually or collectively cause cough (e.g., non-productive cough and chronic cough) or enhance cough sensitivity. In conscious guinea pigs, very low concentrations of inhaled substance P induce cough. Increased levels of substance P are present in nasal epithelial cells of humans with cough hypersensitivity or chronic cough and in the plasma of humans suffering from chronic cough. In addition, tachykinins induce sustained reduction in the activation threshold of spinal integrative neurons, leading to heightened reflex responses to activation of vagal afferent myelinated Aδ-fibers. Moreover, the cough reflex initiated by activation of vagal airway unmyelinated C-fibers is potentiated by release of tachykinins, including substance P, in the NTS. See, e.g., Park 2006, Chapman 1998, LaVinka 2013 and Mazzone 2005.

Neurokinin-1 (NK-1, also called tachykinin receptor 1 or substance P receptor, which binds most strongly to substance P) and neurokinin-2 (NK-2, which binds most strongly to neurokinin A) play an important role in sensitization of the cough reflex. Tachykinin receptors are present on bronchopulmonary C-fibers (Chapman 1998), and NK-1 receptors are expressed in the nucleus tractus solitarius (NTS, also called solitary nucleus or nucleus of the solitary tract) in the brainstem (Bolser 2009). NK-2 receptors are also expressed in the brainstem (Bolser 2009). The NTS is the predominant site of termination of cough-related afferent fibers and is the first synaptic contact of the primary afferent fibers. Substance P released from central terminals of, e.g., vagal C-fibers in the NTS and acting at NK-1 receptors on NTS neurons augments evoked synaptic transmission of bronchopulmonary C-fiber input and hence cough reflex output (Mutoh 2000). Neurokinin-3 (NK-3), which binds most strongly to neurokinin B, is also implicated in cough hypersensitivity (Daoui 1998). Therefore, NK-1 plays an important role in the sensitization of vagal afferent pathways mediating the cough reflex, and activation of NK-1 by substance P can trigger or sensitize the cough reflex.

Through inhibition of NK-1 or/and blockade of binding of substance P to NK-1, an NK-1 antagonist can suppress the cough reflex. By attenuating the activity of afferent nerves that ultimately trigger an urge to cough, an NK-1 antagonist addresses the root cause driving cough hypersensitivity instead of merely suppressing central modulation of the symptom perception. An NK-1 antagonist can act as a peripheral antitussive by inhibiting substance P-induced activation of RAR and C-fiber vagal afferent neurons innervating the airways, including the tracheal and bronchopulmonary epithelium. In addition, an NK-1 antagonist can act as a central antitussive by inhibiting the evoked synaptic transmission of airway sensory input at the NTS in the brainstem, which would normally be augmented by substance P there. For instance, an NK-1 antagonist acting at the NTS can suppress sensitization of the cough reflex caused by activation of tachykinin-containing unmyelinated C-fibers. Therefore, an NK-1 antagonist can block neuronal activation and sensory hyperactivity in the airways (including the trachea and the bronchopulmonary system), which is innervated by vagal afferent nerves, as well as the central cough reflex via the NTS in the medulla oblongata, where the vagal afferent pathways terminate.

The present disclosure provides for the use of an NK-1 antagonist to treat, including alleviate, attenuate and suppress, cough and urge to cough, symptoms and complications thereof, the frequency, severity and impact thereof, and neuronal hypersensitivity underlying cough and urge to cough. The cough can be any and all types of cough, whether the cough is characterized by its duration (e.g., acute cough present for less than 3 weeks, subacute cough present between 3 and 8 weeks, and chronic cough present for more than 8 weeks), its quality (e.g., non-productive [dry] cough), its timing (e.g., cough occurring only during the day [daytime cough] or awake hours [awake cough], cough occurring only at night [nocturnal cough] or during sleep [sleep cough], and cough occurring both during the day and at night or during awake hours and sleep [24-hour cough]) or its character (e.g., a barky cough [such as that associated with croup] and staccato cough [such as that associated with chlamydia pneumonia]), or by any other characterization, including without limitation cough associated with post-nasal drip, post-infectious cough (e.g., post-viral cough), atopic cough, iatrogenic cough (induced by, e.g., endobronchial sutures or a medication such as an angiotensin-converting enzyme [ACE] inhibitor), cough of unknown cause (idiopathic cough or unexplained cough), treatment-resistant (or refractory) cough, and psychogenic cough (habit cough or tic cough).

Furthermore, the cough or urge to cough can be associated with any and all types of medical conditions, including acute and chronic medical conditions and including without limitation respiratory conditions {e.g., inflammatory respiratory conditions (e.g., airway inflammation, asthma [including allergic asthma and cough-variant asthma], acute and chronic bronchitis [including bacterial bronchitis and non-asthmatic eosinophilic bronchitis (NAEB)], chronic obstructive pulmonary disease [COPD, including emphysema], pneumonia [including bacterial and viral pneumonia], pneumonitis [including hypersensitivity pneumonitis], reactive airway disease [e.g., reactive airway dysfunction syndrome (RADS), asthma, COPE), upper respiratory tract infections (including viral URTIs)], acute respiratory distress syndrome [ARDS], etc.), bronchospasm, cough hypersensitivity syndrome (CHS), croup (laryngotracheobronchitis), pulmonary aspiration, respiratory syncytial virus (RSV) infections, rhinitis (including allergic rhinitis and rhinitis due to environmental irritants), sinusitis, rhinosinusitis, tracheobronchitis, tuberculosis, upper airway cough syndrome (UACS, aka post-nasal drip syndrome [PNDS]), upper respiratory tract infections (URTIs, including common cold [viral rhinosinusitis], influenza, bacterial sinusitis and pertussis [whooping cough]), cough associated with smoking, and cough associated with air pollution}, lung tissue disorders {including bronchiectasis, cystic fibrosis, interstitial lung diseases (including pulmonary fibrosis and idiopathic pulmonary fibrosis [IPF]), benign and malignant lung tumors (including alveolar cell carcinoma, bronchogenic carcinoma and non-small cell lung cancer), and sarcoidosis}, benign and malignant airway tumors, benign and malignant tumors in the vicinity of the lungs (e.g., mediastinal tumors), gastroesophageal reflux disease (GERD), sensory neuropathic disorders (neurogenic cough) (including Tourette syndrome), cardiovascular diseases (including aortic aneurysm, heart failure [including congestive heart failure and left ventricular heart failure], and pulmonary infarction), and the other cough-associated medical conditions described herein. A lung tissue disorder can also be regarded as a respiratory condition.

An acute cough can be of sudden onset and can result from an acute disease (e.g., an acute viral URTI, a cold or a flu), and often disappears when the underlying cause (e.g., a cold or a flu) is eliminated. A subacute cough often remains after the underlying cause (e.g., an infection, such as a viral or bacterial infection) is eliminated (e.g., a post-infectious cough, such as a post-viral or post-bacterial cough as in pertussis [whooping cough]). A post-infectious cough is typically a non-productive (dry) cough that produces no phlegm and may be caused by inflammation—the repetition of coughing produces inflammation that causes discomfort, which in turn triggers more coughing. The most common cause of an acute or subacute cough is a viral respiratory tract infection (RTI). In certain embodiments, an NK-1 antagonist is used to treat acute or subacute cough associated with a viral or bacterial RTI or URTI (e.g., common cold [viral rhinosinusitis], influenza, bacterial sinusitis or pertussis).

Chronic cough often is characterized by frequent coughing (e.g., at least 5-10 coughs per hour during daytime or awake hours), and bothersome coughing during sleep. Chronic cough can last for years, including over a decade. Up to about 90% of chronic cough cases in adults are due to post-nasal drip (aka PNDS or UACS, which can be caused by, e.g., an upper airway inflammatory condition such as allergic rhinitis or chronic sinusitis), asthma (e.g., cough-variant asthma), bronchitis (e.g., NAEB) and GERD. The main causes of chronic cough in children are similar with the addition of bacterial bronchitis. Other causes or diagnoses of chronic cough in adults include without limitation foreign material in the airways, post-infectious cough, pertussis, tuberculosis, sarcoidosis, lung cancer, chronic aspiration, bronchiectasis, Zenker's diverticulum (pharyngoesophageal diverticulum), subglottic stenosis, tracheomalacia, tracheoesophageal fistula, trauma, sensory neuropathic disorders (neurogenic cough), psychogenic cough (habit cough), congestive heart failure, and cough induced by ACE inhibitors. In cases of chronic cough that is not obviously caused by an underlying disease or ailment (idiopathic chronic cough), the subject may appear normal in most other respects. In certain embodiments, an NK-1 antagonist is used to treat chronic cough associated with post-nasal drip, asthma (e.g., cough-variant asthma or allergic asthma), bronchitis (e.g., NAEB or bacterial bronchitis), or GERD.

In some embodiments, an NK-1 antagonist is used to treat non-productive (dry) cough. In further embodiments, an NK-1 antagonist is used to treat chronic cough (e.g., non-productive chronic cough, idiopathic chronic cough, refractory/treatment-resistant chronic cough, or daytime, awake or 24-hour chronic cough). Refractory chronic cough (RCC) patients can represent up to about 12% of the general population, can cough frequently while awake (e.g., about 20 or more coughs per hour), and can suffer from RCC for many years. In other embodiments, an NK-1 antagonist is used to treat cough hypersensitivity (e.g., CHS). Most sufferers of chronic cough (e.g., chronic cough associated with a disease) have cough hypersensitivity such that they experience a persistent urge to cough, and innocuous stimuli that would normally not cause coughing often trigger their coughing. In additional embodiments, an NK-1 antagonist is used to treat chronic non-productive cough, sensitized/hypersensitive non-productive cough, or chronic and sensitized/hypersensitive non-productive cough. Without intending to be bound by theory, NK-1 antagonists and the other kinds of antitussives described herein suppress, e.g., non-productive cough and chronic cough by inhibiting the pathway of the unmyelinated C-fibers primarily responsible for such undesired coughs, and permit defensive/protective cough important for maintaining airway patency and preventing pulmonary infection by not inhibiting the pathway of the cough receptor myelinated Aδ-fibers primarily responsible for productive cough.

In some embodiments, treatment with an NK-1 antagonist reduces the frequency (e.g., the number of coughs per hour during daytime, awake hours, sleep or the whole day, which can be objectively monitored with, e.g., VitaloJAK™), the severity (e.g., visual analog scale [VAS] and cough severity diary [CSD]) or the impact (e.g., Leicester cough questionnaire [LCQ] and cough-specific quality of life questionnaire [CQLQ]), or any combination or all thereof, of cough, including acute cough, subacute cough and chronic cough, and urge to cough, by at least about 20%, 30%, 40%, 50%, 60%, 70%, 80%, 90% or 95% (e.g., by at least about 30% or 50%). In some embodiments, treatment with an NK-1 antagonist reduces the frequency, severity or impact, or any combination or all thereof, of cough by about 20-40%, 40-60% or 60-80% or 80-100% (e.g., by about 20-40%).

One or more NK-1 antagonists can be used to treat cough and urge to cough. In some embodiments, the NK-1 antagonist is or comprises a selective NK-1 antagonist. Non-limiting examples of NK-1 antagonists include aprepitant (L-754030 or MK-(0)869), fosaprepitant (L-758298), befetupitant, casopitant (GW-679769), dapitant (RPR-100893), ezlopitant (CJ-11974), lanepitant (LY-303870), maropitant (CJ-11972), netupitant, nolpitantium (SR-140333), orvepitant (GW-823296), rolapitant (SCH-619734), SCH-720881 (active metabolite of rolapitant), serlopitant (MK-(0)594 or VPD-737), tradipitant (VLY-686 or LY-686017), vestipitant (GW-597599), vofopitant (GR-205171), hydroxyphenyl propamidobenzoic acid, maltooligosaccharides (e.g., maltotetraose and maltopentaose), spantides (e.g., spantide I and II), AV-608, AV-818, AZD-2624, BIIF 1149 CL, CGP-49823, CJ-17493, CP-96345, CP-99994, CP-122721, DNK-333, FK-224, FK-888, GR-82334, GR-205171, GSK-424887, HSP-117, KRP-103, L-703606, L-733060, L-736281, L-759274, L-760735, LY-686017, M516102, MDL-105212, MK-0303 (L-001182885), MK-8478 (L-001983867), NKP-608, R-116031, R-116301, RP-67580, S-41744, SCH-206272, SCH-388714, SCH-900978, SLV-317, SSR-240600, T-2328, TA-5538, TAK-637, TKA-731, WIN-51708, ZD-4974, ZD-6021, cycloalkyl (including cyclopentyl, cyclohexyl and cycloheptyl) tachykinin receptor antagonists disclosed in U.S. Pat. No. 5,750,549, hydroxymethyl ether hydroisoindoline tachykinin receptor antagonists disclosed in U.S. Pat. No. 8,124,633, and analogs, derivatives, prodrugs, metabolites and salts thereof.

In some embodiments, the NK-1 antagonist is or comprises serlopitant (described in greater detail below), or a pharmaceutically acceptable salt, solvate, hydrate, clathrate, polymorph, prodrug or metabolite thereof. In other embodiments, the NK-1 antagonist is or comprises MK-0303 (L-001182885) or a pharmaceutically acceptable salt, solvate, hydrate, clathrate, polymorph, prodrug or metabolite thereof. In yet other embodiments, the NK-1 antagonist is or comprises MK-8478 (L-001983867) or a pharmaceutically acceptable salt, solvate, hydrate, clathrate, polymorph, prodrug or metabolite thereof.

In further embodiments, the NK-1 antagonist is or comprises a cycloalkyl (e.g., cyclopentyl, cyclohexyl or cycloheptyl) tachykinin receptor antagonist disclosed in U.S. Pat. No. 5,750,549. In certain embodiments, the NK-1 antagonist is or comprises a cyclopentyl tachykinin receptor antagonist disclosed in U.S. Pat. No. 5,750,549.

In additional embodiments, the NK-1 antagonist is or comprises a hydroxymethyl ether hydroisoindoline tachykinin receptor antagonist disclosed in U.S. Pat. No. 8,124,633. In certain embodiments, the NK-1 antagonist is or comprises the compound designated “Ex. #8” or the compound designated “Ex. #10” in U.S. Pat. No. 8,124,633, or a pharmaceutically acceptable salt, solvate, hydrate, clathrate, polymorph, prodrug or metabolite thereof.

In some embodiments, the NK-1 antagonist is not, or does not comprise, aprepitant, maropitant, nolpitantium (SR-140333), orvepitant, rolapitant, SCH-720881 (active metabolite of rolapitant), CP-96345, CP-99994, DNK-333, FK-224, FK-888, MDL-105212, NKP-608, SCH-206272, SCH-900978, SSR-240600; a compound of Formula I or the specific compound of Formula II disclosed in WO 2017/011445 A1; Compound B disclosed in WO 98/27086 A1; a compound of Formula (I) disclosed in WO 96/06094 A1; a compound of Formula (I) disclosed in US 2017/0283434; a compound of Formula I disclosed in U.S. Pat. No. 8,754,216; a compound of Formula I disclosed in U.S. Pat. No. 7,709,641; a compound of Formula I disclosed in U.S. Pat. No. 7,498,438; a compound of Formula (I) disclosed in U.S. Pat. No. 7,354,922; a compound of Formula (I) disclosed in U.S. Pat. No. 7,122,677; a compound of Formula (I) disclosed in U.S. Pat. No. 7,041,682; a compound of Formula (I) disclosed in U.S. Pat. No. 6,878,732; a compound of Formula I disclosed in U.S. Pat. No. 6,635,630; a piperidinyl compound of Formula I disclosed in U.S. Pat. No. 5,789,422; an indolyl compound of Formula I disclosed in U.S. Pat. No. 5,691,362; or Compound 1, 2, 4 or 5 disclosed in U.S. Pat. No. 5,597,845.

The therapeutically effective amount and the frequency of administration of, and the length of treatment with, the NK-1 antagonist (e.g., serlopitant, MK-0303 or MK-8478) to treat cough and urge to cough may depend on various factors, including the nature and severity of the condition, the potency of the NK-1 antagonist, the mode of administration, the age, body weight, general health, gender and diet of the subject, and the response of the subject to the treatment, and can be determined by the treating physician. In some embodiments, a therapeutically effective amount of the NK-1 antagonist (e.g., serlopitant, MK-0303 or MK-8478) for the treatment of cough and urge to cough is about 1-100 mg, 1-50 mg, 1-10 mg, 10-20 mg, 20-30 mg, 30-40 mg, 40-50 mg or 50-100 mg (e.g., per day or per dose), or as deemed appropriate by the treating physician, which can be administered in a single dose or in divided doses. In certain embodiments, the therapeutically effective dose (e.g., per day or per dose) of the NK-1 antagonist (e.g., serlopitant, MK-0303 or MK-8478) for treating cough and urge to cough is about 1-5 mg (e.g., about 1 mg, 2 mg, 3 mg, 4 mg or 5 mg), about 5-10 mg (e.g., about 5 mg, 6 mg, 7 mg, 8 mg, 9 mg or 10 mg), about 10-20 mg (e.g., about 10 mg, 15 mg or 20 mg), about 20-30 mg (e.g., about 20 mg, 25 mg or 30 mg), about 30-40 mg (e.g., about 30 mg, 35 mg or 40 mg), or about 40-50 mg (e.g., about 40 mg, 45 mg or 50 mg), or about 60 mg, 70 mg, 80 mg, 90 mg or 100 mg. In some embodiments, the therapeutically effective dose of the NK-1 antagonist (e.g., serlopitant, MK-0303 or MK-8478) is administered one or more (e.g., 2, 3, 4 or more) times a day, once every two days, once every three days, twice a week or once a week, or as deemed appropriate by the treating physician. In certain embodiments, the therapeutically effective dose of the NK-1 antagonist (e.g., serlopitant, MK-0303 or MK-8478) is administered once or twice daily. In further embodiments, the therapeutically effective dose of the NK-1 antagonist (e.g., serlopitant, MK-0303 or MK-8478) is about 1-5 mg (e.g., about 1 mg, 3 mg or 5 mg) once or twice daily. In other embodiments, the therapeutically effective dose of the NK-1 antagonist (e.g., serlopitant, MK-0303 or MK-8478) is about 5-10 mg (e.g., about 5 mg, 7.5 mg or 10 mg) once or twice daily. The therapeutically effective dose of the NK-1 antagonist (e.g., serlopitant, MK-0303 or MK-8478) can also be less than 1 mg per day or per dose, such as about 0.25 mg, 0.5 mg or 0.75 mg once or twice daily.

The NK-1 antagonist (e.g., serlopitant, MK-0303 or MK-8478) can also be dosed in an irregular manner. For example, the NK-1 antagonist can be administered 1, 2, 3, 4, 5 or more times in a period of 1 day, 2 days, 3 days, 1 week, 2 weeks, 3 weeks or a month in an irregular manner. Furthermore, the NK-1 antagonist (e.g., serlopitant, MK-0303 or MK-8478) can be taken pro re nata (as needed). For instance, the NK-1 antagonist can be administered 1, 2, 3, 4, 5 or more times, whether in a regular or irregular manner, until cough and urge to cough are alleviated. Once relief from cough and urge to cough is achieved, dosing of the NK-1 antagonist can optionally be discontinued. If cough or urge to cough returns, administration of the NK-1 antagonist, whether in a regular or irregular manner, can be resumed. The appropriate dosage of, frequency of dosing of and length of treatment with the NK-1 antagonist can be determined by the treating physician.

The length of treatment of cough or urge to cough with the NK-1 antagonist can be based on, e.g., the nature of the cough or the cough-associated condition. In certain embodiments, a therapeutically effective amount of the NK-1 antagonist (e.g., serlopitant, MK-0303 or MK-8478) is administered over a period of at least about 1 (e.g., a single dose for the entire therapy), 2 or 3 days, or 1 or 2 weeks, to treat acute cough. In other embodiments, a therapeutically effective amount of the NK-1 antagonist (e.g., serlopitant, MK-0303 or MK-8478) is administered over a period of at least about 3 weeks, 4 weeks (1 month), 5 weeks, 6 weeks or 7 weeks (e.g., at least about 3 weeks or 6 weeks) to treat subacute cough. In further embodiments, a therapeutically effective amount of the NK-1 antagonist (e.g., serlopitant, MK-0303 or MK-8478) is administered over a period of at least about 8 weeks (2 months), 3 months, 4 months, 5 months, 6 months, 1 year, 2 years, 3 years, 5 years, 10 years or longer (e.g., at least about 2 months, 3 months or 6 months) to treat chronic cough.

The NK-1 antagonist (e.g., serlopitant, MK-0303 or MK-8478) can be administered via any suitable route. Potential routes of administration of the NK-1 antagonist include without limitation oral, parenteral (including intramuscular, subcutaneous, intradermal, intravascular, intravenous, intraarterial, intraperitoneal, intramedullary, intrathecal and topical), intracavitary, and topical (including dermal/epicutaneous, transdermal, mucosal, transmucosal, intranasal [e.g., by nasal spray or drop], pulmonary [e.g., by oral or nasal inhalation], buccal, sublingual, rectal [e.g., by suppository] and vaginal [e.g., by suppository]). In some embodiments, the NK-1 antagonist (e.g., serlopitant, MK-0303 or MK-8478) is administered orally (e.g., as a tablet or capsule, optionally with an enteric coating). In other embodiments, the NK-1 antagonist (e.g., serlopitant, MK-0303 or MK-8478) is administered parenterally (e.g., intravenously, subcutaneously or intramuscularly). In certain embodiments, the NK-1 antagonist (e.g., serlopitant, MK-0303 or MK-8478) is administered as an intramuscular depot, which can allow for less frequent dosing of the NK-1 antagonist, such as once every two weeks, monthly or longer.

In further embodiments, the NK-1 antagonist (e.g., serlopitant, MK-0303 or MK-8478) is administered pulmonarily (e.g., by oral or nasal inhalation). In still further embodiments, the NK-1 antagonist (e.g., serlopitant, MK-0303 or MK-8478) is administered intranasally (e.g., by nasal spray, nose drop or pipette). Pulmonary or intranasal administration can allow the NK-1 antagonist to more quickly block neuronal activation and sensory hyperactivity in the airways (including the trachea and the bronchopulmonary system), which is innervated by vagal afferent nerves, as well as the central cough reflex via the nucleus tractus solitarius in the cough center in the medulla oblongata, where vagal afferent nerves terminate. In certain embodiments, a therapeutically effective dose of the NK-1 antagonist is administered pulmonarily or intranasally one or two times daily. In other embodiments, a therapeutically effective dose of the NK-1 antagonist is administered pulmonarily or intranasally three or four times daily. In additional embodiments, the NK-1 antagonist (e.g., serlopitant, MK-0303 or MK-8478) is administered by another topical route (e.g., dermally/epicutaneously, transdermally, mucosally, transmucosally, buccally or sublingually).

For the treatment of chronic cough (e.g., idiopathic chronic cough or refractory/treatment-resistant chronic cough), in some embodiments the NK-1 antagonist (e.g., serlopitant, MK-0303 or MK-8478) is administered in a dose of about 1-5 mg (e.g., about 1, 3 or 5 mg) or about 5-10 mg (e.g., about 5, 7.5 or 10 mg) once or twice daily orally (e.g., as a tablet or capsule) or by inhalation (oral or nasal) for at least about 2 months, 3 months, 6 months, 1 year, 2 years, 3 years or longer (e.g., at least about 2 months, 3 months or 6 months).

The NK-1 antagonist (e.g., serlopitant, MK-0303 or MK-8478) can be administered at any time convenient to the patient. However, NK-1 antagonists may cause drowsiness. To avoid or minimize drowsiness or dizziness during the day, the NK-1 antagonist (e.g., serlopitant, MK-0303 or MK-8478) can be administered shortly before the patient goes to bed. Accordingly, in certain embodiments the NK-1 antagonist (e.g., serlopitant, MK-0303 or MK-8478) is administered at bedtime (e.g., once daily at bedtime). Administration of the NK-1 antagonist at bedtime can also aid with sleep and reduce nighttime coughing. In other embodiments the NK-1 antagonist (e.g., serlopitant, MK-0303 or MK-8478) is administered in the morning (e.g., once daily in the morning).

In additional embodiments, the NK-1 antagonist (e.g., serlopitant, MK-0303 or MK-8478) is administered without food. In some embodiments, the NK-1 antagonist (e.g., serlopitant, MK-0303 or MK-8478) is administered at least about 1 or 2 hours before or after a meal at any time of the day. In certain embodiments, the NK-1 antagonist (e.g., serlopitant, MK-0303 or MK-8478) is administered at least about 2 hours after an evening meal, or at least about 2 hours before or after a meal in the morning. The NK-1 antagonist (e.g., serlopitant, MK-0303 or MK-8478) can also be administered substantially concurrently with food, such as within about 1 hour, 30 minutes or 15 minutes before or after a meal, or with a meal, at any time of the day.

In some embodiments where a more rapid establishment of a therapeutic level of the NK-1 antagonist (e.g., serlopitant, MK-0303 or MK-8478) is desired, the NK-1 antagonist is administered under a dosing schedule in which a loading dose is administered, followed by (i) one or more additional loading doses and then one or more therapeutically effective maintenance doses, or (ii) one or more therapeutically effective maintenance doses without an additional loading dose, as deemed appropriate by the treating physician. A loading dose of a drug is typically larger (e.g., about 1.5, 2, 3, 4 or 5 times larger) than a subsequent maintenance dose and is designed to establish a therapeutic level of the drug more quickly. The one or more therapeutically effective maintenance doses can be any therapeutically effective dose described herein. In certain embodiments, the loading dose is about three times greater than the maintenance dose. In some embodiments, a loading dose of the NK-1 antagonist (e.g., serlopitant, MK-0303 or MK-8478) is administered, followed by administration of a maintenance dose of the NK-1 antagonist after an appropriate time (e.g., after about 12 hr or 24 hr) and thereafter for the duration of therapy—e.g., a loading dose of the NK-1 antagonist is administered on day 1 and a maintenance dose is administered on day 2 and thereafter for the duration of therapy. In some embodiments, the NK-1 antagonist (e.g., serlopitant, MK-0303 or MK-8478) is administered in a loading dose of about 3-15 mg or 15-30 mg once or twice on day 1, followed by a maintenance dose of about 1-5 mg or 5-10 mg once or twice daily for at least about 2 weeks, 1 month, 2 months, 3 months, 6 months, 1 year, 2 years, 3 years or longer (e.g., at least about 1, 2 or 3 months), where the loading dose is three times larger than the maintenance dose and the NK-1 antagonist is administered orally (e.g., as a tablet or capsule), pulmonarily (e.g., by oral or nasal inhalation) or intranasally (e.g., by nasal spray or drop).

In other embodiments, a first loading dose of the NK-1 antagonist (e.g., serlopitant, MK-0303 or MK-8478) is administered on day 1, a second loading dose is administered on day 2, and a maintenance dose is administered on day 3 and thereafter for the duration of therapy. In certain embodiments, the first loading dose is about three times greater than the maintenance dose, and the second loading dose is about two times greater than the maintenance dose.

In some embodiments, an additional antitussive agent is used in combination with an NK-1 antagonist (e.g., serlopitant, MK-0303 or MK-8478) for the treatment of cough or urge to cough, or the cough-associated medical condition, as described elsewhere herein.

Besides reducing peripheral and central neuronal cough hypersensitivity, NK-1 antagonists (including serlopitant, MK-0303 and MK-8478) can exert other beneficial effects. For example, inflammation, which exacerbates coughing and is an important underlying factor in many cough-associated respiratory disorders, is curtailed by NK-1 antagonists. In addition, NK-1 antagonists have antidepressant property, which may aid in treating, e.g., a neurogenic cough.

The disclosure provides a method of treating cough or a cough-associated medical condition, comprising administering to a subject in need of treatment a therapeutically effective amount of an NK-1 antagonist (e.g., serlopitant, MK-0303 or MK-8478), optionally in combination with an additional antitussive agent. The therapeutically effective amount and the frequency of administration of, and the length of treatment with, an NK-1 antagonist (e.g., serlopitant, MK-0303 or MK-8478) to treat cough and urge to cough described herein also apply to treatment of a cough-associated medical condition with the NK-1 antagonist. The disclosure further provides an NK-1 antagonist (e.g., serlopitant, MK-0303 or MK-8478), or a composition comprising an NK-1 antagonist, for use in the treatment of cough or a cough-associated medical condition, optionally in combination with an additional antitussive agent. In addition, the disclosure provides for the use of an NK-1 antagonist (e.g., serlopitant, MK-0303 or MK-8478) in the preparation of a medicament for the treatment of cough or a cough-associated medical condition, optionally in combination with an additional antitussive agent.

### Neurokinin-1 Antagonists

As described above, the disclosure provides for the use of one or more NK-1 antagonists in the treatment of cough (including acute, subacute and chronic cough) and urge to cough. In some embodiments, the NK-1 antagonist is or includes a selective NK-1 antagonist. Non-limiting examples of NK-1 antagonists include aprepitant (L-754030 or MK-(0)869), fosaprepitant (L-758298), befetupitant, casopitant (GW-679769), dapitant (RPR-100893), ezlopitant (CJ-11974), lanepitant (LY-303870), maropitant (CJ-11972), netupitant, nolpitantium (SR-140333), orvepitant (GW-823296), rolapitant (SCH-619734), SCH-720881 (active metabolite of rolapitant), serlopitant (MK-(0)594 or VPD-737), tradipitant (VLY-686 or LY-686017), vestipitant (GW-597599), vofopitant (GR-205171), hydroxyphenyl propamidobenzoic acid, maltooligosaccharides (e.g., maltotetraose and maltopentaose), spantides (e.g., spantide I and II), AV-608, AV-818, AZD-2624, BIIF 1149 CL, CGP-49823, CJ-17493, CP-96345, CP-99994, CP-122721, DNK-333, FK-224, FK-888, GR-82334, GR-205171, GSK-424887, HSP-117, KRP-103, L-703606, L-733060, L-736281, L-759274, L-760735, LY-686017, M516102, MDL-105212, MK-0303 (L-001182885), MK-8478 (L-001983867), NKP-608, R-116031, R-116301, RP-67580, S-41744, SCH-206272, SCH-388714, SCH-900978, SLV-317, SSR-240600, T-2328, TA-5538, TAK-637, TKA-731, WIN-51708, ZD-4974, ZD-6021, cycloalkyl (including cyclopentyl, cyclohexyl and cycloheptyl) tachykinin receptor antagonists disclosed in U.S. Pat. No. 5,750,549, hydroxymethyl ether hydroisoindoline tachykinin receptor antagonists disclosed in U.S. Pat. No. 8,124,633, and analogs, derivatives, prodrugs, metabolites and salts thereof.

MK-0303 (L-001182885) and MK-8478 (L-001983867) have the following structures:

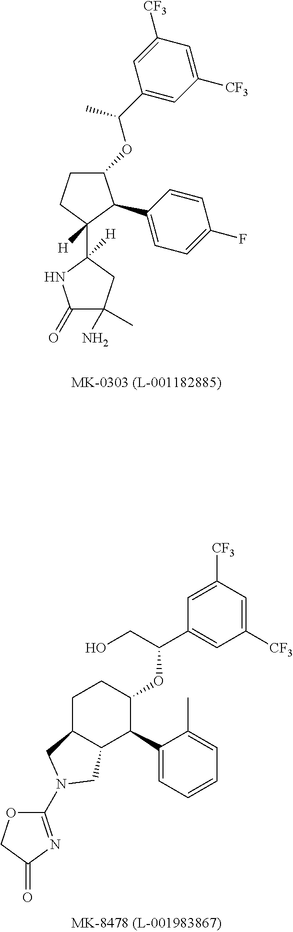 [SMILES: C[C@@H](O[C@H]1CC[C@@H]([C@H]2CC(C)(N)C(=O)N2)[C@@H]1c1ccc(F)cc1)c1cc(C(F)(F)F)cc(C(F)(F)F)c1 ; Cc1ccccc1[C@H]1[C@@H]2CN(C3=NC(=O)CO3)C[C@H]2CC[C@@H]1O[C@H](CO)c1cc(C(F)(F)F)cc(C(F)(F)F)c1]

In some embodiments, the NK-1 antagonist is or includes serlopitant, or a pharmaceutically acceptable salt, solvate, hydrate, clathrate, polymorph, prodrug or metabolite thereof. In other embodiments, the NK-1 antagonist is or includes MK-0303 (L-001182885) or a pharmaceutically acceptable salt, solvate, hydrate, clathrate, polymorph, prodrug or metabolite thereof. In yet other embodiments, the NK-1 antagonist is or includes MK-8478 (L-001983867) or a pharmaceutically acceptable salt, solvate, hydrate, clathrate, polymorph, prodrug or metabolite thereof.

In further embodiments, the NK-1 antagonist is or includes a cycloalkyl (e.g., cyclopentyl, cyclohexyl or cycloheptyl) tachykinin receptor antagonist disclosed in U.S. Pat. No. 5,750,549. In certain embodiments, the NK-1 antagonist is or includes a cyclopentyl tachykinin receptor antagonist disclosed in U.S. Pat. No. 5,750,549. Examples of cycloalkyl tachykinin receptor antagonists disclosed in U.S. Pat. No. 5,750,549 include, but are not limited to:
1-(S)-(1-(R)-(3,5-bis(trifluoromethyl)phenyl)ethoxy)-2-(S)-(4-fluorophenyl)-3-(R)-(2-methoxyethylamino)cyclopentane;
1-(S)-(1-(R)-(3,5-bis(trifluoromethyl)phenyl)ethoxy)-2-(S)-(4-fluorophenyl)-3-(R)—(N-(aminocarbonylmethyl)-N-(2-methoxyethyl)-amino)cyclopentane;
methyl 3-(S)-((2-methoxy-5-(1-tetrazolyl)phenyl)methylamino)-2-(S)-(4-fluorophenyl)cyclopentane-1-(S)-carboxylate;
1-(S)-((2-methoxy-5-(1-tetrazolyl)phenyl)methylamino)-2-(S)-(4-fluorophenyl)-3-(S)-(aminocarbonyl)cyclopentane;
1-(S)-((2-methoxy-5-(1-tetrazolyl)phenyl)methylamino)-2-(S)-(4-fluorophenyl)-3-(S)-(dimethylaminocarbonyl)cyclopentane;
1-(S)-((2-methoxy-5-(1-tetrazolyl)phenyl)methylamino)-2-(S)-(4-fluorophenyl)-3-(S)-(morpholin-4-ylcarbonyl)cyclopentane;
1-(S)-((2-methoxy-5-(1-tetrazolyl)phenyl)methylamino)-2-(S)-(4-fluorophenyl)-3-(S)-(t-butylaminocarbonyl)cyclopentane;
1-(S)-((2-methoxy-5-(1-tetrazolyl)phenyl)methylamino)-2-(S)-(4-fluorophenyl)-3-(S)-(aminocarbonylmethylamino)cyclopentane;
1-(S)-((2-methoxy-5-(1-tetrazolyl)phenyl)methylamino)-2-(S)-(4-fluorophenyl)-3-(S)-(methoxycarbonylamino)cyclopentane;
1-(S)-((2-methoxy-5-(1-tetrazolyl)phenyl)methylamino)-2-(S)-(4-fluorophenyl)-3-(S)-(dimethylaminocarbonylamino)cyclopentane;
1-(S)-((2-methoxy-5-(1-tetrazolyl)phenyl)methylamino)-2-(S)-(4-fluorophenyl)-3-(S)-(methylaminocarbonylamino)cyclopentane;
1-(S)-((2-methoxy-5-(1-tetrazolyl)phenyl)methylamino)-2-(S)-(4-fluorophenyl)-3-(S)-(ethylsulfonylamino)cyclopentane;
1-(S)-((2-methoxy-5-(1-tetrazolyl)phenyl)methylamino)-2-(S)-(4-fluorophenyl)-3-(S)-(pyrrolidin-1-ylmethyl)cyclopentane;
1-(S)-((2-methoxy-5-(5-trifluoromethyltetrazol-1-yl)phenyl)methylamino)-2-(S)-(4-fluorophenyl)-3-(S)-(pyrrolidin-1-ylmethyl)cyclopentane;
1-(S)-((2-methoxy-5-(1-tetrazolyl)phenyl)methylamino)-2-(S)-(4-fluorophenyl)-3-(S)-(1,2,3-triazol-1-ylmethyl)cyclopentane;
1-(S)-((2-methoxy-5-(5-trifluoromethyltetrazol-1-yl)phenyl)methylamino)-2-(S)-(4-fluorophenyl)-3-(S)-(2-methyl-5-tetrazol-5-ylmethyl)cyclopentane;
methyl 3-(SR)-(2-methoxy-5-((5-trifluoromethyl)tetrazol-1-yl)phenyl)methylamino-2-(SR)-(4-fluorophenyl)-cyclopentane-1-(SR)-carboxylate;
N-((2-methoxy-5-trifluoromethoxy)phenylmethyl)-3-(SR)-(5-methyl-1,3,4-oxadiazol-2-yl)-2-(SR)-(4-fluorophenyl)cyclopentan-1-(SR)-amine;
methyl 3-(S)-[2-isopropoxy-5-(1-methyl-1H-tetrazol-5-yl)-phenyl]-methylamino-2-(S)-(4-fluorophenyl)-cyclopentane-1-(S)-carboxylate;
3-(SR)-((2-isopropoxy-5-(5-trifluoromethyltetrazol-1-yl) phenyl)methylamino)-2-(SR)-(4-fluorophenyl)cyclopentane-1-(SR)carboxamide;
methyl 3-(SR)-((2-cyclobutyloxy-5-(1-tetrazolyl)phenyl)methyl-amino)-2-(SR)-(4-fluorophenyl)cyclopentane-1-(SR)carboxylate;
3-(SR)-((2-isopropoxy-5-(tetrazol-1-yl)phenyl)methylamino)-2-(SR)-(4-fluorophenyl)cyclopentane-1-(SR)carboxamide;
1S-(1′S-methyl-(3,5-bistrifluoromethyl)benzyloxy)-2S-phenyl-3R-hydroxymethylcyclohexane;
1S-((1′R-(3,5-bistrifluoromethyl)phenyl)ethoxy)-2S-phenyl-3 S—(N-methyl-N-(5-oxo-1,2,4-triazol-2-yl)methylamino))-cyclohexane;
1S-((1′R-(3,5-bistrifluoromethyl)phenyl)ethoxy)-2S-phenyl-3 S—(N-methyl-N-(5-(1,2,4-triazolylmethyl)amino))-cyclohexane;
1S-((1′R-(3,5-bistrifluoromethyl)phenyl)ethoxy)-2S-phenyl-3 S-aminocyclohexane;
1S-(1′R-(3,5-bistrifluoromethyl)phenyl)ethoxy)-2S-phenyl-3 S-(amino-aminocarbonyl methyl amino-cyclohexane;
1S-(1′R-(3,5-bistrifluoromethyl)phenyl)ethoxy)-2S-phenyl-3 S—(N-(2-pyrrolidinone-5-(S)-yl-methyl))aminocyclohexane;
1S—(N-2-methoxy-5-(5-trifluoromethyl-1,2,3,4-tetrazol-1-yl))benzyl amino-2S-phenyl-3S-hydroxymethylcyclohexane;
1S—(N-2-methoxy-5-(1,2,3,4-tetrazol-1-yl))benzyl amino-2S-phenyl-3S-methylamino-cyclohexane;
1(S)—N-(2-methoxy-5-(trifluoromethyl-1,2,3,4-tetrazol-1-yl))benzyl-2 (S)-phenyl-3 (S)-(pyrrolidin-1-yl-methyl)cyclohexane;
1(S)—N-(2-methoxy-5-(trifluoromethyl-1,2,3,4-tetrazol-1-yl))benzyl-2 (S)-phenyl-3 (S)-methoxymethyl)cyclohexane;
1(S)—N-(2-methoxy-5-(1-tetrazolyl))-benzylamino-2(S)-phenylcyclohexane;
1(S)—N-(2-methoxy-5-(trifluoromethyl-1,2,3,4-tetrazol-1-yl))benzyl-2 (S)-phenylcyclohexane;
1S—[(N-benzyloxycarbonyl)-(N-2-methoxy-5-(5-trifluoro-methyl-1,2,3,4-tetrazol-1-yl))]benzylamino-2S-phenyl-3S-(2-hydroxyethyl)-cyclohexane;
3-(S)-(2-methoxy-5-((5-trifluoromethyl)tetrazol-1-yl)phenyl)-methylamino-2-(S)-(4-fluorophenyl)cyclopentane-1-(S)-(N-t-butyl)carboxamide;
3-(SR)-(2-methoxy-5-((5-trifluoromethyl)tetrazol-1-yl)phenyl)methylamino-2-(SR)-(4-fluorophenyl)-cyclopentane-1-(SR)-(N-t-butyl)carboxamide;
1-(S)-((2-isopropoxy-5-(5-trifluoromethyltetrazol-1-yl)phenyl)methylamino)-2-(S)-(4-fluorophenyl)-3-(S)-(pyrrolidin-1-ylmethyl)cyclopentane;
1-(S)-((2-methoxy-5-(5-trifluoromethyltetrazol-1-yl)phenyl)methylamino)-2-(S)-(4-fluorophenyl)-3-(S)-(2-(S)-(aminocarbonyl)pyrrolidin-1-ylmethyl)cyclopentane;
1-(S)-((2-methoxy-5-(5-trifluoromethyltetrazol-1-yl)phenyl)methylamino)-2-(S)-(4-fluorophenyl)-3-(S)-(1-methyl-5-tetrazol-5-ylmethyl)-cyclopentane;
N-(2-methoxy-5-((5-trifluoromethyl)tetrazol-1-yl)phenyl)methyl)-3-(SR)-(imidazol-2-yl)-2-(SR)-(4-fluorophenyl)cyclopentan-1-(SR)-amine;
N-(2-methoxy-5-((5-trifluoromethyl)tetrazol-1-yl)phenyl)methyl)-3-(SR)-((1-methyl)imidazol-2-yl)-2-(SR)-(4-fluorophenyl)cyclopentan-1-(SR)-amine;
N-(2-methoxy-5-((5-trifluoromethyl)tetrazol-1-yl)phenyl)methyl)-3-(SR)-(thiazol-2-yl)-2-(SR)-(4-fluorophenyl)cyclopentan-1-(SR)-amine;
N-(2-methoxy-5-((5-trifluoromethyl)tetrazol-1-yl)phenyl)methyl)-3-(S)-(thiazol-2-yl)-2-(S)-(4-fluorophenyl)cyclopentan-1-(S)-amine;
N-(2-methoxy-5-((5-trifluoromethyl)tetrazol-1-yl)phenyl)methyl)-3-(SR)-(isoxazol-3-yl)-2-(SR)-(4-fluorophenyl)cyclopentan-1-(SR)-amine;
N-(2-methoxy-5-((5-trifluoromethyl)tetrazol-1-yl)phenyl)methyl)-3-(S)-(5-methyl-1,3,4-oxadiazol-2-yl)-2-(S)-(4-fluorophenyl)cyclopentan-1-(S)-amine;
N-(2-methoxy-5-((5-trifluoromethyl)tetrazol-1-yl)phenyl)methyl)-3-(SR)-(tetrazol-1-yl)-2-(RS)-(4-fluorophenyl)cyclopentan-1-(SR)-amine;
N-(2-methoxy-5-((5-trifluoromethyl)tetrazol-1-yl)phenyl)methyl)-3-(SR)-(1,2,4-triazol-4-yl)-2-(SR)-(4-fluorophenyl)cyclopentan-1-(SR)-amine;
(1RS,2RS,3RS)-3-((5-(3,5-dimethylisoxazol-4-yl)-2-methoxyphenyl)methylamino)-2-(4-fluorophenyl)cyclopentane-carboxylic acid methyl ester;
methyl 3-(S,R)-((2-methoxy-5-(5-trifluoromethyl-tetrazol-1-yl)-3-pyridine)methylamino)-2-(S,R)-(4-fluorophenyl)cyclopentane-1-(S,R)-carboxylate;
methyl 3-(S)-(5-(5-trifluoromethyl-1-tetrazol-1-yl)-(7-benzofuran)methylamino)-2-(S,R)-(4-fluorophenyl)cyclopentane-1-(S,R)-carboxylate;
methyl 3-(S)-[(5-cyano-2-isopropoxy-phenyl)-methylamino]-2-(S)-(4-fluorophenyl)-cyclopentane-1-(S)-carboxylate;
1-(S)-[(5-cyano-2-isopropoxy-phenyl)-methylamino]-2-(S)-(4-fluorophenyl)-3-(S)-(2-thiazol-2-yl)-cyclopentane;
methyl 3-(SR)-((2-isopropoxy-5-(tetrazol-1-yl)phenyl)methylamino)-2-(SR)-(4-fluorophenyl)cyclopentane-1-(SR)-carboxylate;
3-(SR)-((2-isopropoxy-5-(tetrazol-1-yl)phenyl)methylamino)-2-(SR)-(4-fluorophenyl)cyclopentane-1-(SR)-tert-butyl-carboxamide;
methyl 3-(SR)-((2-isopropoxy-5-(5-trifluoromethyltetrazol-1-yl)phenyl)methylamino)-2-(SR)-(4-fluorophenyl)cyclopentane-1-(SR)-carboxylate;
methyl 3-(S)-((2-methylsulfanyl-5-(5-trifluoromethyltetrazol-1-yl)phenyl)methylamino)-2-(S)-(4-fluorophenyl)cyclopentane-1-(S)-carboxylate;
1(S)—N-(2-methoxy-5-(1-tetrazolyl))-benzylamino-2(S)-phenyl-3(S)-carboxymethylcyclohexane;
l(S)—N-(2-methoxy-5-(trifluoromethyl-1,2,3,4-tetrazol-1-yl))benzyl-2(S)-phenyl-3(S)-imidazolecyclohexane;
1(S)—N-(2-methoxy-5-(1-tetrazolyl))-benzylamino-2(S)-phenyl-3(S)-ethylcyclohexane; and

enantiomers and racemic mixtures thereof.

In additional embodiments, the NK-1 antagonist is or includes a hydroxymethyl ether hydroisoindoline tachykinin receptor antagonist disclosed in U.S. Pat. No. 8,124,633. Examples of hydroxymethyl ether hydroisoindoline tachykinin receptor antagonists disclosed in U.S. Pat. No. 8,124,633 include, but are not limited to:
tert-butyl (3aR,4R,5S,7aS)-5-{(1S)-1-[3,5-bis(trifluoromethyl)phenyl]-2-hydroxyethoxy}-4-(2-methylphenyl)octahydro-2H-isoindole-2-carboxylate;
tert-butyl (3aR,4R,5S,7aS)-5-{(1R)-1-[3,5-bis(trifluoromethyl)phenyl]-2-hydroxyethoxy}-4-(2-methylphenyl)octahydro-2H-isoindole-2-carboxylate;
(2S)-2-[3,5-bis(trifluoromethyl)phenyl]-2-{[(3aR,4R,5S,7aS)-4-(2-methylphenyl)octahydro-1H-isoindol-5-yl]oxy}ethanol;
3-[(3aR,4R,5S,7aS)-5-{(1S)-1-[3,5-bis(trifluoromethyl)phenyl]-2-hydroxyethoxy}-4-(2-methylphenyl)octahydro-2H-isoindol-2-yl]cyclopent-2-en-1-one;
3-[(3aR,4R,5S,7aS)-5-{(1S)-1-[3,5-bis(trifluoromethyl)phenyl]-2-methoxyethoxy}-4-(2-methylphenyl)octahydro-2H-isoindol-2-yl]cyclopent-2-en-1-one;
2-[(3aR,4R,5S,7aS)-5-{(1S)-1-[3,5-bis(trifluoromethyl)phenyl]-2-hydroxyethoxy}-4-(2-methylphenyl)octahydro-2H-isoindol-2-yl]-1,3-oxazol-4(5H)-one (MK-8478);
(3aR,4R,5 S,7aS)-5-{(1S)-1-[3,5-bis(trifluoromethyl)phenyl]-2-hydroxyethoxy}-N,N-dimethyl-4-(2-methylphenyl)octahydro-2H-isoindole-2-carboxamide;
(2S)-2-[3,5-bis(trifluoromethyl)phenyl]-2-{[(3aR,4R,5S,7aS)-2-isobutyryl-4-(2-methylphenyl)octahydro-1H-isoindol-5-yl]oxy}ethanol;

the compound designated “Ex. #8”;

the compound designated “Ex. #9”;

the compound designated “Ex. #10”;

the compound designated “Ex. #11”;

the compound designated “Ex. #12”;

the compound designated “Ex. #13”;

the compound designated “Ex. #14”;

the compound designated “Ex. #15”;

the compound designated “Ex. #16”;

the compound designated “Ex. #17”;

the compound designated “Ex. #18”;

the compound designated “Ex. #19”;

the compound designated “Ex. #20”;

the compound designated “Ex. #21”;

the compound designated “Ex. #22”;

the compound designated “Ex. #23”;

the compound designated “Ex. #24”;

the compound designated “Ex. #25”;

the compound designated “Ex. #26”;

the compound designated “Ex. #27”; and

enantiomers and racemic mixtures thereof.

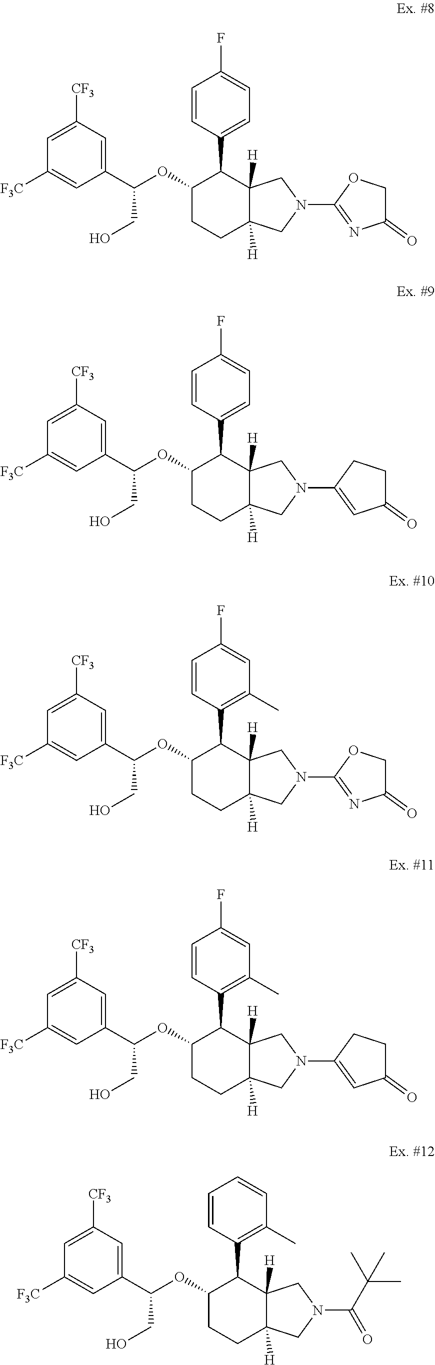 [SMILES: O=C1COC(N2C[C@H]3CC[C@H](O[C@H](CO)c4cc(C(F)(F)F)cc(C(F)(F)F)c4)[C@@H](c4ccc(F)cc4)[C@@H]3C2)=N1 ; O=C1C=C(N2C[C@H]3CC[C@H](O[C@H](CO)c4cc(C(F)(F)F)cc(C(F)(F)F)c4)[C@@H](c4ccc(F)cc4)[C@@H]3C2)CC1 ; Cc1cc(F)ccc1[C@H]1[C@@H]2CN(C3=NC(=O)CO3)C[C@H]2CC[C@@H]1O[C@H](CO)c1cc(C(F)(F)F)cc(C(F)(F)F)c1 ; Cc1cc(F)ccc1[C@H]1[C@@H]2CN(C3=CC(=O)CC3)C[C@H]2CC[C@@H]1O[C@H](CO)c1cc(C(F)(F)F)cc(C(F)(F)F)c1 ; Cc1ccccc1[C@H]1[C@@H]2CN(C(=O)C(C)(C)C)C[C@H]2CC[C@@H]1O[C@H](CO)c1cc(C(F)(F)F)cc(C(F)(F)F)c1]

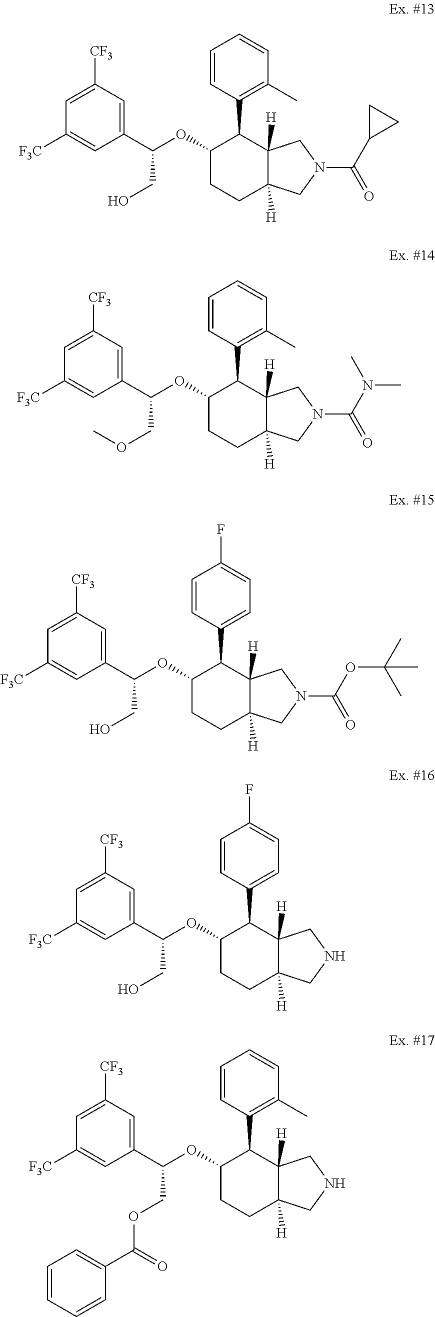

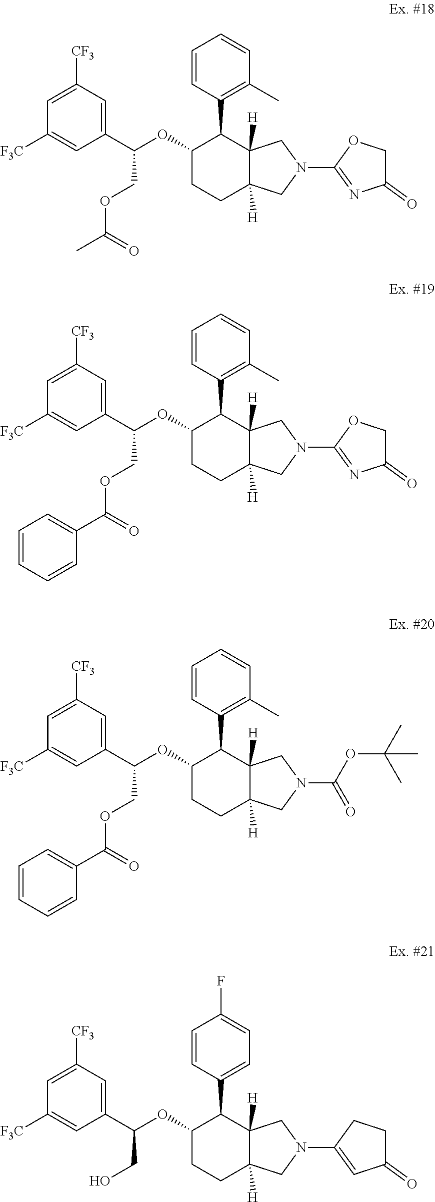 [SMILES: CC(=O)OC[C@@H](O[C@H]1CC[C@@H]2CN(C3=NC(=O)CO3)C[C@H]2[C@@H]1c1ccccc1C)c1cc(C(F)(F)F)cc(C(F)(F)F)c1 ; Cc1ccccc1[C@H]1[C@@H]2CN(C3=NC(=O)CO3)C[C@H]2CC[C@@H]1O[C@H](COC(=O)c1ccccc1)c1cc(C(F)(F)F)cc(C(F)(F)F)c1 ; Cc1ccccc1[C@H]1[C@@H]2CN(C(=O)OC(C)(C)C)C[C@H]2CC[C@@H]1O[C@H](COC(=O)c1ccccc1)c1cc(C(F)(F)F)cc(C(F)(F)F)c1 ; O=C1C=C(N2C[C@H]3CC[C@H](O[C@@H](CO)c4cc(C(F)(F)F)cc(C(F)(F)F)c4)[C@@H](c4ccc(F)cc4)[C@@H]3C2)CC1]

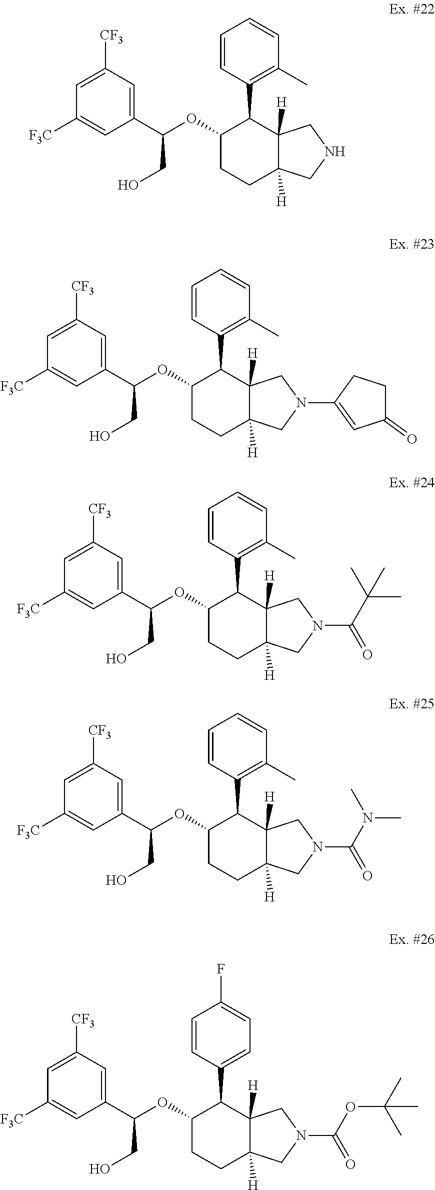

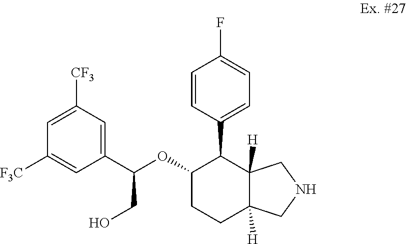 [SMILES: OC[C@H](O[C@H]1CC[C@@H]2CNC[C@H]2[C@@H]1c1ccc(F)cc1)c1cc(C(F)(F)F)cc(C(F)(F)F)c1]

In certain embodiments, the NK-1 antagonist is or includes the compound designated “Ex. #8” or the compound designated “Ex. #10” in U.S. Pat. No. 8,124,633, or a pharmaceutically acceptable salt, solvate, hydrate, clathrate, polymorph, prodrug or metabolite thereof.

In other embodiments, the NK-1 antagonist is or includes aprepitant or fosaprepitant, or a pharmaceutically acceptable salt, solvate, hydrate, clathrate, polymorph, prodrug or metabolite thereof. In additional embodiments, the NK-1 antagonist is or includes befetupitant, or a pharmaceutically acceptable salt, solvate, hydrate, clathrate, polymorph, prodrug or metabolite thereof. In further embodiments, the NK-1 antagonist is or includes casopitant, or a pharmaceutically acceptable salt, solvate, hydrate, clathrate, polymorph, prodrug or metabolite thereof. In still further embodiments, the NK-1 antagonist is or includes dapitant, or a pharmaceutically acceptable salt, solvate, hydrate, clathrate, polymorph, prodrug or metabolite thereof. In yet further embodiments, the NK-1 antagonist is or includes ezlopitant, or a pharmaceutically acceptable salt, solvate, hydrate, clathrate, polymorph, prodrug or metabolite thereof. In other embodiments, the NK-1 antagonist is or includes lanepitant, or a pharmaceutically acceptable salt, solvate, hydrate, clathrate, polymorph, prodrug or metabolite thereof. In still other embodiments, the NK-1 antagonist is or includes maropitant, or a pharmaceutically acceptable salt, solvate, hydrate, clathrate, polymorph, prodrug or metabolite thereof. In yet other embodiments, the NK-1 antagonist is or includes netupitant, or a pharmaceutically acceptable salt, solvate, hydrate, clathrate, polymorph, prodrug or metabolite thereof.

In further embodiments, the NK-1 antagonist is or includes nolpitantium, or a pharmaceutically acceptable salt, solvate, hydrate, clathrate, polymorph, prodrug or metabolite thereof. In still further embodiments, the NK-1 antagonist is or includes orvepitant, or a pharmaceutically acceptable salt, solvate, hydrate, clathrate, polymorph, prodrug or metabolite thereof. In yet further embodiments, the NK-1 antagonist is or includes rolapitant or SCH-720881 (active metabolite of rolapitant), or a pharmaceutically acceptable salt, solvate, hydrate, clathrate, polymorph, prodrug or metabolite thereof. In other embodiments, the NK-1 antagonist is or includes tradipitant, or a pharmaceutically acceptable salt, solvate, hydrate, clathrate, polymorph, prodrug or metabolite thereof. In still other embodiments, the NK-1 antagonist is or includes vestipitant, or a pharmaceutically acceptable salt, solvate, hydrate, clathrate, polymorph, prodrug or metabolite thereof. In yet other embodiments, the NK-1 antagonist is or includes vofopitant, or a pharmaceutically acceptable salt, solvate, hydrate, clathrate, polymorph, prodrug or metabolite thereof.

In some embodiments, the NK-1 antagonist is not, or does not include, aprepitant, maropitant, nolpitantium (SR-140333), orvepitant, rolapitant, SCH-720881 (active metabolite of rolapitant), CP-96345, CP-99994, DNK-333, FK-224, FK-888, MDL-105212, NKP-608, SCH-206272, SCH-900978, SSR-240600; a compound of Formula I or the specific compound of Formula II disclosed in WO 2017/011445 A1; Compound B disclosed in WO 98/27086 A1; a compound of Formula (I) disclosed in WO 96/06094 A1; a compound of Formula (I) disclosed in US 2017/0283434; a compound of Formula I disclosed in U.S. Pat. No. 8,754,216; a compound of Formula I disclosed in U.S. Pat. No. 7,709,641; a compound of Formula I disclosed in U.S. Pat. No. 7,498,438; a compound of Formula (I) disclosed in U.S. Pat. No. 7,354,922; a compound of Formula (I) disclosed in U.S. Pat. No. 7,122,677; a compound of Formula (I) disclosed in U.S. Pat. No. 7,041,682; a compound of Formula (I) disclosed in U.S. Pat. No. 6,878,732; a compound of Formula I disclosed in U.S. Pat. No. 6,635,630; a piperidinyl compound of Formula I disclosed in U.S. Pat. No. 5,789,422; an indolyl compound of Formula I disclosed in U.S. Pat. No. 5,691,362; or Compound 1, 2, 4 or 5 disclosed in U.S. Pat. No. 5,597,845.

### Description of Serlopitant

Serlopitant is a potent and highly selective antagonist of neurokinin-1 (also called substance P receptor). By binding to and not activating NK-1, serlopitant inhibits actions of substance P, including activation of cough neurons in the airways and the brainstem and incitement of inflammation.

Serlopitant has the structure shown below. The IUPAC name for serlopitant is 3-R3aR,4R,5S,7aS)-5-[(1R)-1-[3,5-bis(trifluoromethyl)phenyl]ethoxyl-4-(4-fluorophenyl)-1,3,3a,4,5,6,7,7a-octahydroisoindol-2-yl]cyclopent-2-en-1-one. The USAN name for serlopitant is 3-[(3aR,4R,5S,7aS)-5-R1R)-1-[3,5-bis(trifluoromethyl)phenyl]ethoxyl-4-(4-fluorophenyl)octahydro-2H-isoindol-2-yl]cyclopent-2-en-1-one. The disclosure also encompasses all stereoisomers of serlopitant, including both enantiomers and all diastereomers of serlopitant in substantially pure form and mixtures of both enantiomers (including a racemic mixture) and mixtures of two or more diastereomers of serlopitant in any ratio. The disclosure further encompasses all isotopically enriched forms of serlopitant, including without limitation those enriched in the content of ²H (deuterium), ¹³C, ¹⁵N, ¹⁷O, ¹⁸O or ¹⁹F, or any combination thereof, at one or more, or all, instances of the corresponding atom(s). Moreover, the disclosure encompasses any and all salt forms of serlopitant. Various methods of synthesizing serlopitant are known in the art. See, e.g., Jiang et al., J. Med. Chem., 52:3039-3046 (2009); U.S. Pat. No. 7,544,815; and U.S. Pat. No. 7,217,731.

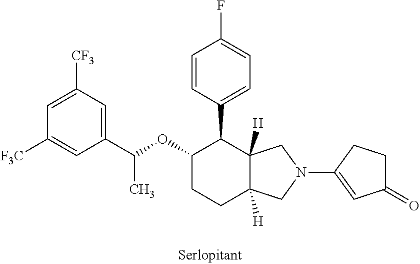

Whether as a free base or a salt, serlopitant can exist unsolvated or unhydrated, or solvated or hydrated. Solvated forms of serlopitant can be formed with a pharmaceutically acceptable solvent, such as water or ethanol. In certain embodiments, serlopitant, whether as a free base or a salt, is used substantially unhydrated.

The disclosure also encompasses polymorphs (crystalline forms) of serlopitant. Examples of polymorphs of serlopitant include without limitation anhydrous crystalline Forms I and II of free base serlopitant as disclosed in US 2009/0270477. Form I is characterized by diffraction peaks obtained from X-ray powder diffraction pattern corresponding to d-spacings of 10.4, 9.9, 9.2, 5.5, 5.0, 4.1, 3.9, 3.6 and 3.5 angstroms. Form II is characterized by diffraction peaks obtained from X-ray powder diffraction pattern corresponding to d-spacings of 7.7, 5.3, 4.9, 4.8, 4.6, 4.2, 3.9, 3.8 and 2.8 angstroms. Form I is thermodynamically more stable below 70° C. and is non-hygroscopic under all tested relative humidity conditions. In certain embodiments, serlopitant is used in the form of polymorph Form I.

### Stereoisomers

It is understood that the present disclosure encompasses all possible stereoisomers, including both enantiomers and all possible diastereomers in substantially pure form and mixtures of both enantiomers in any ratio (including a racemic mixture of enantiomers) and mixtures of two or more diastereomers in any ratio, of the compounds described herein, including without limitation NK-1 antagonists (e.g., serlopitant, MK-0303 and MK-8478), and not only the specific stereoisomers as indicated by drawn structure or nomenclature. Some embodiments of the disclosure relate to the specific stereoisomers indicated by drawn structure or nomenclature. If the phrase “or stereoisomers thereof” or the like with respect to a compound is recited in certain instances of the disclosure, such recitation shall not be interpreted as an intended omission of any of the other possible stereoisomers of the compound in other instances of the disclosure where the compound is mentioned without recitation of the phrase “or stereoisomers thereof” or the like, unless stated otherwise or the context clearly indicates otherwise.

### Salt Forms of Drug Substances

Drug substances (e.g., NK-1 antagonists, such as serlopitant, MK-0303 or MK-8478) may exist in a non-salt form (e.g., a free base or a free acid, or having no basic or acidic atom or functional group) or as salts if they can form salts. Drug substances that can form salts can be used in the non-salt form or in the form of pharmaceutically acceptable salts. If a drug has, e.g., a basic nitrogen atom, the drug can form an addition salt with an acid (e.g., a mineral acid [such as HCl, HBr, HI, nitric acid, phosphoric acid or sulfuric acid] or an organic acid [such as a carboxylic acid or a sulfonic acid]). Suitable acids for use in the preparation of pharmaceutically acceptable salts include without limitation acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, D-gluconic acid, D-glucuronic acid, L-glutamic acid, alpha-oxo-glutaric acid, glycolic acid, hippuric acid, hydrobromic acid, hydrochloric acid, hydroiodic acid, (±)-DL-lactic acid, (+)-L-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, propionic acid, L-pyroglutamic acid, pyruvic acid, saccharic acid, salicylic acid, 4-amino-salicylic acid, sebacic acid, stearic acid, succinic acid, sulfuric acid, tannic acid, (±)-DL-tartaric acid, (+)-L-tartaric acid, thiocyanic acid, p-toluenesulfonic acid, undecylenic acid, and valeric acid.

If a drug has an acidic group (e.g., a carboxyl group), the drug can form an addition salt with a base. Pharmaceutically acceptable base addition salts can be formed with, e.g., metals (e.g., alkali metals or alkaline earth metals) or amines (e.g., organic amines). Non-limiting examples of metals useful as cations include alkali metals (e.g., lithium, sodium, potassium and cesium), alkaline earth metals (e.g., magnesium and calcium), aluminum and zinc. Metal cations can be provided by way of, e.g., inorganic bases, such as hydroxides, carbonates and hydrogen carbonates. Non-limiting examples of organic amines useful for forming base addition salts include chloroprocaine, choline, cyclohexylamine, dibenzylamine, N,N′-dibenzylethylenediamine, dicyclohexylamine, diethanolamine, ethylenediamine, N-ethylpiperidine, histidine, isopropylamine, N-methylglucamine, procaine, pyrazine, triethylamine and trimethylamine Pharmaceutically acceptable salts are discussed in detail in Handbook of Pharmaceutical Salts, Properties, Selection and Use, P. Stahl and C. Wermuth, Eds., Wiley-VCH (2011).

### Pharmaceutical Compositions

To treat cough or urge to cough, an NK-1 antagonist (e.g., serlopitant, MK-0303 or MK-8478) can be administered alone or in the form of a pharmaceutical composition. In some embodiments, a pharmaceutical composition comprises an NK-1 antagonist (e.g., serlopitant, MK-0303 or MK-8478) or a pharmaceutically acceptable salt, solvate, hydrate, clathrate, polymorph, prodrug or metabolite thereof, and one or more pharmaceutically acceptable carriers or excipients. The composition can optionally contain an additional therapeutic agent as described herein. A pharmaceutical composition contains a therapeutically effective amount of a therapeutic agent (e.g., an NK-1 antagonist, such as serlopitant, MK-0303 or MK-8478) and one or more pharmaceutically acceptable carriers or excipients, and is formulated for administration to a subject for therapeutic use. For purposes of the content of a pharmaceutical composition, the terms “therapeutic agent”, “active ingredient”, “active agent” and “drug” encompass prodrugs.

A pharmaceutical composition contains a therapeutic agent (e.g., an NK-1 antagonist, such as serlopitant, MK-0303 or MK-8478) in substantially pure form. In some embodiments, the purity of the therapeutic agent is at least about 95%, 96%, 97%, 98% or 99%. In certain embodiments, the purity of the therapeutic agent is at least about 98% or 99%. In addition, a pharmaceutical composition is substantially free of contaminants or impurities. In some embodiments, the level of contaminants or impurities other than residual solvent in a pharmaceutical composition is no more than about 5%, 4%, 3%, 2% or 1% relative to the combined weight of the intended active and inactive ingredients. In certain embodiments, the level of contaminants or impurities other than residual solvent in a pharmaceutical composition is no more than about 2% or 1% relative to the combined weight of the intended active and inactive ingredients. Pharmaceutical compositions generally are prepared according to current good manufacturing practice (GMP), as recommended or required by, e.g., the Federal Food, Drug, and Cosmetic Act § 501(a)(2)(B) and the International Conference on Harmonisation Q7 Guideline.

Pharmaceutical compositions/formulations can be prepared in sterile form. For example, pharmaceutical compositions/formulations for parenteral administration by injection or infusion generally are sterile. Sterile pharmaceutical compositions/formulations are compounded or manufactured according to pharmaceutical-grade sterilization standards known to those of skill in the art, such as those disclosed in or required by the United States Pharmacopeia Chapters 797, 1072 and 1211, and 21 Code of Federal Regulations 211.

Pharmaceutically acceptable carriers and excipients include pharmaceutically acceptable materials, vehicles and substances. Non-limiting examples of excipients include liquid and solid fillers, diluents, binders, lubricants, glidants, solubilizers, surfactants, dispersing agents, disintegration agents, emulsifying agents, wetting agents, suspending agents, thickeners, solvents, isotonic agents, buffers, pH adjusters, stabilizers, preservatives, antioxidants, antimicrobial agents, antibacterial agents, antifungal agents, absorption-delaying agents, sweetening agents, flavoring agents, coloring agents, adjuvants, encapsulating materials and coating materials. The use of such excipients in pharmaceutical formulations is known in the art. For example, conventional vehicles and carriers include without limitation oils (e.g., vegetable oils, such as sesame oil), aqueous solvents (e.g., saline, phosphate-buffered saline [PBS] and isotonic solutions [e.g., Ringer's solution]), and solvents (e.g., dimethyl sulfoxide [DMSO] and alcohols [e.g., ethanol, glycerol and propylene glycol]). Except insofar as any conventional carrier or excipient is incompatible with the active ingredient, the disclosure encompasses the use of conventional carriers and excipients in formulations containing a therapeutic agent (e.g., an NK-1 antagonist, such as serlopitant, MK-0303 or MK-8478). See, e.g., Remington: The Science and Practice of Pharmacy, 21st Ed., Lippincott Williams & Wilkins (Philadelphia, Pa. [2005]); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Preformulation and Formulation, Gibson, Ed., CRC Press (Boca Raton, Fla. [2004]).

Proper formulation can depend on various factors, such as the mode of administration chosen. Potential modes of administration of pharmaceutical compositions comprising an NK-1 antagonist (e.g., serlopitant, MK-0303 or MK-8478) include without limitation oral, parenteral (including intramuscular, subcutaneous, intradermal, intravascular, intravenous, intraarterial, intraperitoneal, intramedullary, intrathecal and topical), intracavitary, and topical (including dermal/epicutaneous, transdermal, mucosal, transmucosal, intranasal [e.g., by nasal spray or drop], pulmonary [e.g., by oral or nasal inhalation], buccal, sublingual, rectal [e.g., by suppository], and vaginal [e.g., by suppository]).

As an example, formulations of an NK-1 antagonist (e.g., serlopitant, MK-0303 or MK-8478) suitable for oral administration can be presented as, e.g., boluses; tablets, capsules, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

Tablets can contain an NK-1 antagonist (e.g., serlopitant, MK-0303 or MK-8478) in admixture with, e.g., a filler or inert diluent (e.g., calcium carbonate, calcium phosphate, lactose, mannitol or microcrystalline cellulose), a binding agent (e.g., a starch, gelatin, acacia, alginic acid or a salt thereof, or microcrystalline cellulose), a lubricating agent (e.g., stearic acid, magnesium stearate, talc or silicon dioxide), and a disintegrating agent (e.g., crospovidone, croscarmellose sodium or colloidal silica), and optionally a surfactant (e.g., sodium lauryl sulfate). The tablets can be uncoated or can be coated with, e.g., an enteric coating that protects the active ingredient from the acidic environment of the stomach, or with a material that delays disintegration and absorption of the active ingredient in the gastrointestinal tract and thereby provides a sustained action over a longer time period. In certain embodiments, a tablet comprises an NK-1 antagonist (e.g., serlopitant, MK-0303 or MK-8478), mannitol, microcrystalline cellulose, magnesium stearate, silicon dioxide, croscarmellose sodium and sodium lauryl sulfate, and optionally lactose monohydrate, and the tablet is optionally film-coated (e.g., with Opadry®).

Push-fit capsules or two-piece hard gelatin capsules can contain an NK-1 antagonist (e.g., serlopitant, MK-0303 or MK-8478) in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, an NK-1 antagonist (e.g., serlopitant, MK-0303 or MK-8478) can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Compositions for oral administration can also be formulated as solutions or suspensions in an aqueous liquid or/and a non-aqueous liquid, or as oil-in-water liquid emulsions or water-in-oil liquid emulsions. Dispersible powder or granules of an NK-1 antagonist (e.g., serlopitant, MK-0303 or MK-8478) can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

In some embodiments, an NK-1 antagonist (e.g., serlopitant, MK-0303 or MK-8478) is contained in an amphiphilic vehicle of a liquid or semi-solid formulation for oral administration which provides improved solubility, stability and bioavailability of the NK-1 antagonist, as described in US 2010/0209496. The amphiphilic vehicle contains a solution, suspension, emulsion (e.g., oil-in-water emulsion) or semi-solid mixture of the NK-1 antagonist (e.g., serlopitant, MK-0303 or MK-8478) admixed with liquid or/and semi-solid excipients which fills an encapsulated dosage form (e.g., a hard gelatin capsule or a soft gelatin capsule containing a plasticizer [e.g., glycerol or/and sorbitol]). In some embodiments, the amphiphilic vehicle comprises an amphiphilic agent selected from fatty acid esters of glycerol (glycerin), propylene glycol and sorbitol. In certain embodiments, the amphiphilic agent is selected from mono- and di-glycerides of C₈-C₁₂ saturated fatty acids. In further embodiments, the amphiphilic agent is selected from CAPMUL® MCM, CAPMUL® MCM 8, CAPMUL® MCM 10, IMWITOR® 308, IMWITOR® 624, IMWITOR® 742, IMWITOR® 988, CAPRYOL™ PGMC, CAPRYOL™ 90, LAUROGLYCOL™ 90, CAPTEX® 200, CRILL™ 1, CRILL™ 4, PECEOL® and MAISINE™ 35-1. In some embodiments, the amphiphilic vehicle further comprises propylene glycol, a propylene glycol-sparing agent (e.g., ethanol or/and glycerol), or an antioxidant (e.g., butylated hydroxyanisole, butylated hydroxytoluene, propyl gallate or/and sodium sulfite), or any combination thereof. In additional embodiments, the amphiphilic vehicle contains on a weight basis about 0.1-5% of the NK-1 antagonist (e.g., serlopitant, MK-0303 or MK-8478), about 50-90% of the amphiphilic agent, about 5-40% of propylene glycol, about 5-20% of the propylene glycol-sparing agent, and about 0.01-0.5% of the antioxidant.

An NK-1 antagonist (e.g., serlopitant, MK-0303 or MK-8478) can also be formulated for parenteral administration by injection or infusion to circumvent gastrointestinal absorption and first-pass metabolism. A representative parenteral route is intravenous. Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous or non-aqueous (e.g., oily) sterile injection solutions can contain an NK-1 antagonist (e.g., serlopitant, MK-0303 or MK-8478) along with excipients such as an antioxidant, a buffer, a bacteriostat and solutes that render the formulation isotonic with the blood of the subject. Aqueous or non-aqueous sterile suspensions can contain an NK-1 antagonist (e.g., serlopitant, MK-0303 or MK-8478) along with excipients such as a suspending agent and a thickening agent, and optionally a stabilizer and an agent that increases the solubility of the NK-1 antagonist to allow for the preparation of a more concentrated solution or suspension. As another example, a sterile aqueous solution for injection or infusion (e.g., subcutaneously or intravenously) can contain an NK-1 antagonist (e.g., serlopitant, MK-0303 or MK-8478), NaCl, a buffering agent (e.g., sodium citrate), a preservative (e.g., meta-cresol), and optionally a base (e.g., NaOH) or/and an acid (e.g., HCl) to adjust pH.

For topical administration, an NK-1 antagonist (e.g., serlopitant, MK-0303 or MK-8478) can be formulated as, e.g., a buccal or sublingual tablet or pill. Advantages of a buccal or sublingual tablet or pill include avoidance of first-pass metabolism and circumvention of gastrointestinal absorption. A buccal or sublingual tablet or pill can also be designed to provide faster release of the NK-1 antagonist for more rapid uptake of it into systemic circulation. In addition to a therapeutically effective amount of the NK-1 antagonist (e.g., serlopitant, MK-0303 or MK-8478), the buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

For topical administration, an NK-1 antagonist (e.g., serlopitant, MK-0303 or MK-8478) can also be formulated for intranasal administration. The nasal mucosa provides a big surface area, a porous endothelium, a highly vascular subepithelial layer and a high absorption rate, and hence allows for high bioavailability. Moreover, intranasal administration avoids first-pass metabolism and can introduce a significant concentration of the NK-1 antagonist to the central nervous system, allowing the NK-1 antagonist to block the central cough reflex via the nucleus tractus solitarius in the cough center in the medulla oblongata, where vagal afferent nerves terminate. An intranasal solution or suspension formulation can comprise an NK-1 antagonist (e.g., serlopitant, MK-0303 or MK-8478) along with excipients such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. In certain embodiments, a nasal spray formulation comprises an NK-1 antagonist (e.g., serlopitant, MK-0303 or MK-8478), microcrystalline cellulose, sodium carboxymethylcellulose, dextrose and water, and optionally an acid (e.g., HCl) to adjust pH. An intranasal solution or suspension formulation can be administered to the nasal cavity by any suitable means, including but not limited to a dropper, a pipette, or spray using, e.g., a metering atomizing spray pump.

An additional mode of topical administration is pulmonary, including by oral inhalation and nasal inhalation, which is described in detail below.

Other suitable topical formulations and dosage forms include without limitation ointments, creams, gels, lotions, pastes and the like, as described in Remington: The Science and Practice of Pharmacy, 21st Ed., Lippincott Williams & Wilkins (Philadelphia, Pa. [2005]). Ointments are semi-solid preparations that are typically based on petrolatum or a petroleum derivative. Creams are viscous liquids or semi-solid emulsions, either oil-in-water or water-in-oil. Cream bases are water-washable, and contain an oil phase, an emulsifier and an aqueous phase. The oil phase, also called the “internal” phase, generally comprises petrolatum and a fatty alcohol (e.g., cetyl or stearyl alcohol). The aqueous phase typically, although not necessarily, exceeds the oil phase in volume, and usually contains a humectant. The emulsifier in a cream formulation is generally a non-ionic, anionic, cationic or amphoteric surfactant. Gels are semi-solid, suspension-type systems. Single-phase gels contain organic macromolecules (polymers) distributed substantially uniformly throughout the carrier liquid, which is typically aqueous but can also contain an alcohol (e.g., ethanol or isopropanol) and optionally an oil. Lotions are preparations to be applied to the skin surface without friction, and are typically liquid or semi-liquid preparations in which solid particles, including the active agent, are present in a water or alcohol base. Lotions are usually suspensions of finely divided solids and typically contain suspending agents to produce better dispersion as well as compounds useful for localizing and holding the active agent in contact with the skin. Pastes are semi-solid dosage forms in which the active agent is suspended in a suitable base. Depending on the nature of the base, pastes are divided between fatty pastes or those made from single-phase aqueous gels.

Various excipients can be included in a topical formulation. For example, solvents, including a suitable amount of an alcohol, can be used to solubilize the active agent. Other optional excipients include without limitation gelling agents, thickening agents, emulsifiers, surfactants, stabilizers, buffers, antioxidants, preservatives, cooling agents (e.g. menthol), opacifiers, fragrances and colorants. For an active agent having a low rate of permeation through the skin or mucosal tissue, a topical formulation can contain a permeation enhancer to increase the permeation of the active agent through the skin or mucosal tissue. A topical formulation can also contain an irritation-mitigating excipient that reduces any irritation to the skin or mucosa caused by the active agent, the permeation enhancer or any other component of the formulation.

In some embodiments, an NK-1 antagonist (e.g., serlopitant, MK-0303 or MK-8478) is delivered from a sustained-release composition. As used herein, the term “sustained-release composition” encompasses sustained-release, prolonged-release, extended-release, slow-release and controlled-release compositions, systems and devices. Use of a sustained-release composition can have benefits, such as an improved profile of the amount of the drug or an active metabolite thereof delivered to the target site(s) over a time period, including delivery of a therapeutically effective amount of the drug or an active metabolite thereof over a prolonged time period. In certain embodiments, the sustained-release composition delivers the NK-1 antagonist over a period of at least about 1 day, 2 days, 3 days, 1 week, 2 weeks, 3 weeks, 1 month, 2 months, 3 months or longer. In some embodiments, the sustained-release composition is a drug-encapsulation system, such as nanoparticles, microparticles or a capsule made of, e.g., a biodegradable polymer or/and a hydrogel. In certain embodiments, the sustained-release composition comprises a hydrogel. Non-limiting examples of polymers of which a hydrogel can be composed include polyvinyl alcohol, acrylate polymers (e.g., sodium polyacrylate), and other homopolymers and copolymers having a relatively large number of hydrophilic groups (e.g., hydroxyl or/and carboxylate groups). In other embodiments, the sustained-release drug-encapsulation system comprises a membrane-enclosed reservoir, wherein the reservoir contains a drug and the membrane is permeable to the drug. Such a drug-delivery system can be in the form of, e.g., a transdermal patch.

In some embodiments, the sustained-release composition is an oral dosage form, such as a tablet or capsule. For example, a drug can be embedded in an insoluble porous matrix such that the dissolving drug must make its way out of die matrix before it can be absorbed through the gastrointestinal tract. Alternatively, a drug can be embedded in a matrix that swells to form a gel through which the drug exits. Sustained release can also be achieved by way of a single-layer or multi-layer osmotic controlled-release oral delivery system (OROS). An OROS is a tablet with a semi-permeable outer membrane and one or more small laser-drilled holes in it. As the tablet passes through the body, water is absorbed through the semi-permeable membrane via osmosis, and the resulting osmotic pressure pushes the drug out through the hole(s) in the tablet and into the gastrointestinal tract where it can be absorbed.

In further embodiments, the sustained-release composition is formulated as polymeric nanoparticles or microparticles, wherein the polymeric particles can be delivered, e.g., by inhalation or injection or from an implant. In some embodiments, the polymeric implant or polymeric nanoparticles or microparticles are composed of a biodegradable polymer. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. For example, biodegradable polymeric microspheres composed of polylactic acid or/and polyglycolic acid can serve as sustained-release pulmonary drug-delivery systems. The biodegradable polymer of the polymeric implant or polymeric nanoparticles or microparticles can be selected so that the polymer substantially completely degrades around the time the period of treatment is expected to end, and so that the byproducts of the polymer's degradation, like the polymer, are biocompatible.

For a delayed or sustained release of an NK-1 antagonist (e.g., serlopitant, MK-0303 or MK-8478), a composition can also be formulated as a depot that can be implanted in or injected into a subject, e.g., intramuscularly or subcutaneously. A depot formulation can be designed to deliver the NK-1 antagonist over a longer period of time, e.g., over a period of at least about 1 week, 2 weeks, 3 weeks, 1 month, 6 weeks, 2 months, 3 months or longer. For example, the NK-1 antagonist can be formulated with a polymeric material (e.g., polyethylene glycol [PEG], polylactic acid [PLA] or polyglycolic acid [PGA], or a copolymer thereof [e.g., PLGA]), a hydrophobic material (e.g., as an emulsion in an oil) or/and an ion-exchange resin, or as a sparingly soluble derivative (e.g., a sparingly soluble salt). As an illustrative example, an NK-1 antagonist (e.g., serlopitant, MK-0303 or MK-8478) can be incorporated or embedded in sustained-release microparticles composed of PLGA and formulated as a monthly depot.

An NK-1 antagonist (e.g., serlopitant, MK-0303 or MK-8478) can also be contained or dispersed in a matrix material. The matrix material can comprise a polymer (e.g., ethylene-vinyl acetate) and controls the release of the compound by controlling dissolution or/and diffusion of the compound from, e.g., a reservoir, and can enhance the stability of the compound while contained in the reservoir. Such a release system can be designed as a sustained-release system, can be configured as, e.g., a transdermal or transmucosal patch, and can contain an excipient that can accelerate the compound's release, such as a water-swellable material (e.g., a hydrogel) that aids in expelling the compound out of the reservoir. U.S. Pat. Nos. 4,144,317 and 5,797,898 describe examples of such a release system.

The release system can provide a temporally modulated release profile (e.g., pulsatile release) when time variation in plasma levels is desired, or a more continuous or consistent release profile when a constant plasma level is desired. Pulsatile release can be achieved from an individual reservoir or from a plurality of reservoirs. For example, where each reservoir provides a single pulse, multiple pulses (“pulsatile” release) are achieved by temporally staggering the single pulse release from each of multiple reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of a compound through it over an extended time period. In addition, continuous release can be approximated by releasing several pulses of a compound in rapid succession (“digital” release). An active release system can be used alone or in conjunction with a passive release system, as described in U.S. Pat. No. 5,797,898.

In addition, pharmaceutical compositions comprising an NK-1 antagonist (e.g., serlopitant, MK-0303 or MK-8478) can be formulated as, e.g., liposomes, micelles (e.g., those composed of biodegradable natural or/and synthetic polymers, such as lactosomes), microspheres, microparticles or nanoparticles, whether or not designed for sustained release. For example, liposomes can be used as sustained□release pulmonary drug-delivery systems that deliver drugs to the alveolar surface for treatment of lung diseases and systemic diseases.

The pharmaceutical compositions can be manufactured in any suitable manner known in the art, e.g., by means of conventional mixing, dissolving, suspending, granulating, dragee-making, levigating, emulsifying, encapsulating, entrapping or compressing processes.

A pharmaceutical composition can be presented in unit dosage form as a single dose wherein all active and inactive ingredients are combined in a suitable system, and components do not need to be mixed to form the composition to be administered. The unit dosage form can contain an effective dose, or an appropriate fraction thereof, of a therapeutic agent (e.g., an NK-1 antagonist, such as serlopitant, MK-0303 or MK-8478). Representative examples of a unit dosage form include a tablet, capsule or pill for oral administration, and powder in a vial or ampoule for oral or nasal inhalation.

Alternatively, a pharmaceutical composition can be presented as a kit, wherein the active ingredient, excipients and carriers (e.g., solvents) are provided in two or more separate containers (e.g., ampoules, vials, tubes, bottles or syringes) and need to be combined to form the composition to be administered. The kit can contain instructions for storing, preparing and administering the composition (e.g., a solution to be injected intravenously).

A kit can contain all active and inactive ingredients in unit dosage form or the active ingredient and inactive ingredients in two or more separate containers, and can contain instructions for using the pharmaceutical composition to treat cough or a cough-associated medical condition.

In some embodiments, a kit contains an NK-1 antagonist (e.g., serlopitant, MK-0303 or MK-8478) or a pharmaceutically acceptable salt, solvate, hydrate, clathrate, polymorph, prodrug or metabolite thereof, and instructions for administering the compound to treat cough or a cough-associated medical condition. In certain embodiments, the compound is contained or incorporated in, or provided by, a device or system configured for pulmonary delivery of the compound by oral inhalation, such as a metered-dose inhaler, a dry powder inhaler or a nebulizer.

Inhalation Formulations and Devices

Pulmonary administration can be accomplished by, e.g., oral inhalation or nasal inhalation. Advantages of pulmonary drug delivery include, but are not limited to: 1) avoidance of first pass hepatic metabolism; 2) fast drug action; 3) large surface area of the alveolar region for absorption, high permeability of the lungs (thin air-blood barrier), and profuse vasculature of the airways; 4) smaller doses to achieve equivalent therapeutic effect compared to other oral routes; 5) local action within the respiratory tract; 6) reduced systemic side effects; and 7) reduced extracellular enzyme levels compared to the gastrointestinal tract due to the large alveolar surface area. An advantage of oral inhalation over nasal inhalation includes deeper penetration/deposition of the drug into the lungs. Pulmonary administration, whether by oral or nasal inhalation, can be a suitable route of administration for drugs that are intended to act locally in the lungs or/and systemically, for which the lungs serve as a portal to the systemic circulation. Pulmonary administration allows an NK-1 antagonist to more quickly block neuronal activation and sensory hyperactivity in the airways (including the trachea and the bronchopulmonary system), which is innervated by vagal afferent nerves, as well as the central cough reflex via the nucleus tractus solitarius in the brainstem, where vagal afferent nerves have endings.

Oral or nasal inhalation can be achieved by means of, e.g., a metered-dose inhaler (MDI), a nebulizer or a dry powder inhaler (DPI). For example, an NK-1 antagonist (e.g., serlopitant, MK-0303 or MK-8478) can be formulated for aerosol administration to the respiratory tract by oral or nasal inhalation. The drug is delivered in a small particle size (e.g., between about 0.5 micron and about 5 microns), which can be obtained by micronization, to improve, e.g., drug deposition in the lungs and drug suspension stability. The drug can be provided in a pressurized pack with a suitable propellant, such as a hydrofluoroalkane (HFA, e.g., 1,1,1,2-tetrafluoroethane [HFA-134a]), a chlorofluorocarbon (CFC, e.g., dichlorodifluoromethane, trichlorofluoromethane or dichlorotetrafluoroethane), or a suitable gas (e.g., oxygen, compressed air or carbon dioxide). The drug in the aerosol formulation is dissolved, or more often suspended, in the propellant for delivery to the lungs. The aerosol can contain excipients such as a surfactant (which enhances penetration into the lungs by reducing the high surface tension forces at the air-water interface within the alveoli, may also emulsify, solubilize or/and stabilize the drug, and can be, e.g., a phospholipid such as lecithin) or/and a stabilizer. For example, an MDI formulation can comprise an NK-1 antagonist (e.g., serlopitant, MK-0303 or MK-8478), a propellant (e.g., an HFA such as 1,1,1,2-tetrafluoroethane), a surfactant (e.g., a fatty acid such as oleic acid), and a co-solvent (e.g., an alcohol such as ethanol). The MDI formulation can optionally contain a dissolved gas (e.g., CO₂). After device actuation, the bursting of CO₂ bubbles within the emitted aerosol droplets breaks up the droplets into smaller droplets, thereby increasing the respirable fraction of drug. As another example, a nebulizer formulation can comprise an NK-1 antagonist (e.g., serlopitant, MK-0303 or MK-8478), a surfactant (e.g., a Tween® such as polysorbate 80), a chelator or preservative (e.g., edetate disodium), an isotonicity agent (e.g., sodium chloride), pH buffering agents (e.g., citric acid/sodium citrate), and water. The drug can be delivered by means of, e.g., a nebulizer or an MDI with or without a spacer, and the drug dose delivered can be controlled by a metering chamber (nebulizer) or a metering valve (MDI).

Metered-dose inhalers (also called pressurized metered-dose inhalers [pMDI]) are the most widely used inhalation devices. A metering valve delivers a precise amount of aerosol (e.g., about 20-100 μL) each time the device is actuated. MDIs typically generate aerosol faster than the user can inhale, which can result in deposition of much of the aerosol in the mouth and the throat. The problem of poor coordination between device actuation and inhalation can be addressed by using, e.g., a breath-actuated MDI or a coordination device. A breath-actuated MDI (e.g., Easibreathe®) is activated when the device senses the user's inspiration and discharges a drug dose in response. The inhalation flow rate is coordinated through the actuator and the user has time to actuate the device reliably during inhalation. In a coordination device, a spacer (or valved holding chamber), which is a tube attached to the mouthpiece end of the inhaler, serves as a reservoir or chamber holding the drug that is sprayed by the inhaler and reduces the speed at which the aerosol enters the mouth, thereby allowing for the evaporation of the propellant from larger droplets. The spacer simplifies use of the inhaler and increases the amount of drug deposited in the lungs instead of in the upper airways. The spacer can be made of an anti-static polymer to minimize electrostatic adherence of the emitted drug particles to the inner walls of the spacer.

Nebulizers generate aerosol droplets of about 1-5 microns. They do not require user coordination between device actuation and inhalation, which can significantly affect the amount of drug deposited in the lungs. Compared to MDIs and DPIs, nebulizers can deliver larger doses of drug, albeit over a longer administration time. Examples of nebulizers include without limitation human-powered nebulizers, jet nebulizers (e.g., AeroEclipse® II BAN [breath-actuated], CompAIR™ NE-C801 [virtual valve], PARI LC® Plus [breath-enhanced] and SideStream Plus [breath-enhanced]), ultrasonic wave nebulizers, and vibrating mesh nebulizers (e.g., Akita2® Apixneb, I-neb AAD System with metering chambers, MicroAir® NE-U22, Omron U22 and PARI eFlow® rapid). As an example, a pulsed ultrasonic nebulizer can aerosolize a fixed amount of the drug per pulse, and can comprise an opto-acoustical trigger that allows the user to synchronize each breath to each pulse.

Respimat® Soft Mist™ inhaler combines advantages of an MDI and a nebulizer. It is a small, hand-held inhaler that does not need a power supply (like an MDI) and slowly aerosolizes a propellant-free drug solution as a soft mist (like a nebulizer), thereby reducing drug deposition in the oropharyngeal region and increasing drug deposition in the central and peripheral lung regions. The Soft Mist™ inhaler can create a large fraction of respirable droplets with slow velocity from a metered volume of drug solution. A drug delivered from the Soft Mist™ inhaler can potentially achieve the same therapeutic outcome at a significantly lower dose compared to delivery from an MDI.

For oral or nasal inhalation using a dry powder inhaler (DPI), an NK-1 antagonist (e.g., serlopitant, MK-0303 or MK-8478) can be provided in the form of a dry micronized powder, where the drug particles are of a certain small size (e.g., between about 0.5 micron and about 5 microns) to improve, e.g., aerodynamic properties of the dispersed powder and drug deposition in the lungs. Particles between about 0.5 micron and about 5 microns deposit by sedimentation in the terminal bronchioles and the alveolar regions. By contrast, the majority of larger particles (>5 microns) do not follow the stream of air into the many bifurcations of the airways, but rather deposit by impaction in the upper airways, including the oropharyngeal region of the throat. A DPI formulation can contain the drug particles alone or blended with a powder of a suitable larger base/carrier, such as lactose, starch, a starch derivative (e.g., hydroxypropylmethyl cellulose) or polyvinylpyrrolidine. The carrier particles enhance flow, reduce aggregation, improve dose uniformity and aid in dispersion of the drug particles. A DPI formulation can optionally contain an excipient such as magnesium stearate or/and leucine that improves the performance of the formulation by interfering with inter-particle bonding (by anti-adherent action). The powder formulation can be provided in unit dose form, such as a capsule (e.g., a gelatin capsule) or a cartridge in a blister pack, which can be manually loaded or pre-loaded in an inhaler. The drug particles can be drawn into the lungs by placing the mouthpiece or nosepiece of the inhaler into the mouth or nose, taking a sharp, deep inhalation to create turbulent airflow, and holding the breath for a period of time (e.g., about 5-10 seconds) to allow the drug particles to settle down in the bronchioles and the alveolar regions. When the user actuates the DPI and inhales, airflow through the device creates shear and turbulence, inspired air is introduced into the powder bed, and the static powder blend is fluidized and enters the user's airways. There, the drug particles separate from the carrier particles due to turbulence and are carried deep into the lungs, while the larger carrier particles impact on the oropharyngeal surfaces and are cleared. Thus, the user's inspiratory airflow achieves powder de-agglomeration and aeroionisation, and determines drug deposition in the lungs. (While a passive DPI requires rapid inspiratory airflow to de-agglomerate drug particles, rapid inspiration is not recommended with an MDI or nebulizer, since it creates turbulent airflow and fast velocity which increase drug deposition by impaction in the upper airways.) Compared to an MDI, a DPI (including a passive, breath-activated DPI) can potentially deliver larger doses of drug, and larger-size drugs (e.g., macromolecules), to the lungs.

Lactose (e.g., alpha-lactose monohydrate) is the most commonly used carrier in DPI formulations. Examples of grades/types of lactose monohydrate for DPI formulations include without limitation DCL 11, Flowlac® 100, Inhalac® 230, Lactohale® 300, Lactopress® SD 250 (spray-dried lactose), Respitose® SV003 and Sorbolac® 400. A DPI formulation can contain a single lactose grade or a combination of different lactose grades. For example, a fine lactose grade like Lactohale® 300 or Sorbolac® 400 may not be a suitable DPI carrier and may need to be blended with a coarse lactose grade like DCL 11, Flowlac® 100, Inhalac® 230 or Respitose® SV003 (e.g., about a 1:9 ratio of fine lactose to coarse lactose) to improve flow. Tables 7 and 8 show non-limiting examples of grades/types of lactose that can be used in DPI formulations. The distribution of the carrier particle sizes affects the fine particle fraction/dose (FPF or FPD) of the drug, with a high FPF being desired for drug delivery to the lungs. FPF/FPD is the respirable fraction/dose mass out of the DPI device with an aerodynamic particle size ≤5 microns in the inspiration air. High FPF, and hence good DPI performance, can be obtained from, e.g., DPI formulations having an approximately 1:9 ratio of fine lactose (e.g., Lactohale® 300) to coarse lactose (e.g., Respitose® SV003) and about 20% w/w overages to avoid deposition of the drug in the capsule shell or the DPI device and to deliver essentially all of the drug to the airways.

TABLE 7

| | | Range of Particle Sizes (μm) | | |
|---|---|---|---|---|
| Product | Type | 10% | 50% | 90% |
| Lactohale ® | LH200 | <9 | <69 | <141 |
| InhaLac ® | 230 | <35 | <93 | <138 |
| Respitose ® | ML001 | <4 | <43 | <146 |
| | ML003 | <4 | <35 | <106 |
| | SV003 | <30 | <59 | <90 |
| | SV004 | <32 | <61 | <93 |

TABLE 8

| | | Range of Particle Sizes | | | |
|---|---|---|---|---|---|
| Product | Type | <45 μm | <100 μm | <150 μm | <250 μm |
| Respitose ® | ML003 | 65% | 98% | 100% | NA |
| Respitose ® | ML002 | 65% | 98% | NA | 100% |

Other carriers for DPI formulations include without limitation glucose, mannitol (e.g., crystallized mannitol [Pearlitol 110 C] and spray-dried mannitol [Pearlitol 100 SD]), maltitol (e.g., crystallized maltitol [Maltisorb P90]), sorbitol and xylitol.

To improve the performance of DPI formulations, pulmospheres can be used. These relatively large porous, hollow particles have low particle density and improved dispersibility. Pulmospheres can be prepared using a polymeric or non-polymeric excipient by, e.g., solvent evaporation or spray drying. For example, pulmospheres can be made of phosphatidylcholine, the primary component of human lung surfactant. The relatively large size of pulmospheres allows them to remain in the alveolar region longer than their non-porous counterparts by avoiding phagocytic clearance. Pulmospheres can also be used in aerosol formulations for MDIs as well as for DPIs.

Dry powder inhalers can be classified by dose type into single-unit dose (including disposable and reusable) and multi-dose (including multi-dose reservoirs and multi-unit dose). In a single-unit dose DPI, the formulation can be a powder mix of a micronized drug powder and a carrier and can be supplied in individual capsules, which are inserted into the inhaler for a single dose and are removed and discarded after use. The capsule body containing the dose falls into the device, while the cap is retained in the entry port for subsequent disposal. As the user inhales, the portion of the capsule containing the drug experiences erratic motion in the airstream, causing dislodged particles to be entrained and subsequently inhaled. Particle de-aggregation is caused mainly by turbulence promoted by the grid upstream of the mouthpiece or nosepiece. Examples of single-unit dose DPIs include without limitation Aerolizer®, AIR®, Conix One® (foil seal), Diskhaler®, Diskus®, Handihaler®, Microhaler®, Rotahaler® and Turbospin®.

A multi-unit dose DPI uses factory-metered and -sealed doses packaged in a manner so that the device can hold multiple doses without the user having to reload. The packaging typically contains replaceable disks or cartridges, or strips of foil-polymer blister packaging that may or may not be reloadable. For example, individual doses can be packaged in blister packs on a disk cassette. Following piercing, inspiratory flow through the packaging depression containing the drug induces dispersion of the powder. The aerosol stream is mixed with a bypass flow entering through holes in the mouthpiece or nosepiece, which gives rise to turbulence and promotes particle de-agglomeration. Advantages of the pre-packaging include protection from the environment until use and ensurance of adequate control of dose uniformity. Examples of multi-unit dose DPIs include without limitation Acu-Breath®, Bulkhaler®, Certihaler®, DirectHaler®, Diskhaler®, Diskus®, Dispohaler®, MF-DPI®, Miat-Haler®, NEXT DPI®, Prohaler®, Swinhaler® and Technohaler®.

A multi-dose reservoir DPI stores the formulation in bulk, and has a built-in mechanism to meter individual doses from the bulk upon actuation. It contains multiple doses of small pellets of micronized drug that disintegrate into their primary particles during metering and inhalation. One dose can be dispensed into the dosing chamber by a simple back-and-forth twisting action on the base of the reservoir. Scrapers actively force the drug into conical holes, which causes the pellets to disintegrate. Fluidization of the powder is achieved by shear force as air enters the inhaler, and particle de-agglomeration occurs via turbulence. Advantages of multi-dose reservoir DPIs include their relative ease and low cost of manufacture, and the ease of inclusion of a large number of doses within the device. Examples of multi-dose reservoir DPIs include without limitation Acu-Breath®, Airmax®, Bulkhaler®, Certihaler®, Clickhaler®, Cyclovent®, Dispohaler®, JAGO®, MF-DPI®, Miat-Haler®, NEXT DPI®, Swinhaler® and Turbuhaler®.

Most DPIs are breath-activated (“passive”), relying on the user's inhalation for aerosol generation. Examples of passive DPIs include without limitation Airmax®, Novolizer®, Otsuka DPI (compact cake), and the DPIs mentioned above. The air classifier technology (ACT) is an efficient passive powder dispersion mechanism employed in DPIs. In ACT, multiple supply channels generate a tangential airflow that results in a cyclone within the device during inhalation. There are also power-assisted (“active”) DPIs (based on, e.g., pneumatics, impact force or vibration) that use energy to aid, e.g., particle de-agglomeration. For example, the active mechanism of Exubera® inhalers utilizes mechanical energy stored in springs or compressed-air chambers. Examples of active DPIs include without limitation Actispire® (single-unit dose), Aspirair® (multi-dose), Exubera® (single-unit dose), MicroDose® (multi-unit dose and electronically activated), Omnihaler® (single-unit dose), Pfeiffer DPI (single-unit dose), and Spiros® (multi-unit dose).

### Topical Compositions Comprising an NK-1 Antagonist

Topical formulations for application to the mucosa or skin can be useful for transmucosal or transdermal administration of an active agent to the local tissue underlying the mucosa (e.g., bronchial mucosa, esophageal mucosa and nasal mucosa) and into the blood for systemic distribution. Advantages of topical administration can include circumvention of the gastrointestinal tract (including enzymes and acid in the GI tract and absorption through it) and hepatic first-pass metabolism; delivery of an active agent with a short half-life, a small therapeutic index or/and low oral bioavailability; controlled, continuous and sustained release of the active agent; a more uniform plasma level or delivery profile of the active agent; lower dose and less frequent dosing of the active agent; reduction of systemic side effects (e.g., side effects caused by a temporary overdose or an overly high peak plasma drug concentration); minimal or no invasiveness; ease of self-administration; and increased patient compliance.

In general and in addition to the disclosure on topical formulations described elsewhere herein, compositions suitable for topical administration include without limitation liquid or semi-liquid preparations such as sprays, gels, liniments, lotions, oil-in-water or water-in-oil emulsions such as creams, foams, ointments and pastes, and solutions or suspensions such as drops (e.g., nose drops). In some embodiments, a topical composition comprises an active agent dissolved, dispersed or suspended in a carrier. The carrier can be in the form of, e.g., a solution, a suspension, an emulsion, an ointment or a gel base, and can contain, e.g., petrolatum, lanolin, a wax (e.g., bee wax), mineral oil, a long-chain alcohol, polyethylene glycol or polypropylene glycol, a diluent (e.g., water or/and an alcohol [e.g., ethanol or propylene glycol]), a gel, an emulsifier, a thickening agent, a stabilizer or a preservative, or a combination thereof. A topical composition can include, or a topical formulation can be administered by means of, e.g., a transmucosal or transdermal delivery device, such as a transmucosal or transdermal patch, a microneedle patch or an iontophoresis device. A topical composition can deliver the active agent transmucosally or transdermally via a concentration gradient (with or without the use of a chemical permeation enhancer) or an active mechanism (e.g., iontophoresis).

Representative kinds of topical compositions are described below for purposes of illustration.

I. Topical Compositions Comprising a Permeation Enhancer

In some embodiments, a topical composition comprises an NK-1 antagonist (e.g., serlopitant, MK-0303 or MK-8478) and a permeation enhancer. The composition can optionally contain an additional therapeutic agent. In certain embodiments, the composition contains the NK-1 antagonist (e.g., serlopitant, MK-0303 or MK-8478) in free base form.

The permeation enhancer increases the permeability of the mucosa or skin to the therapeutic agent(s). In certain embodiments, the permeation enhancer is N-lauroyl sarcosine, sodium octyl sulfate, methyl laurate, isopropyl myristate, oleic acid, glyceryl oleate or sodium lauryl sulfoacetate, or a combination thereof. In certain embodiments, the composition contains on a weight/volume (w/v) basis the permeation enhancer in an amount of about 1-20%, 1-15%, 1-10% or 1-5%. To enhance further the ability of the therapeutic agent(s) to penetrate the skin or mucosa, the composition can also contain a surfactant, an azone-like compound, an alcohol, a fatty acid or ester, or an aliphatic thiol.

The composition can further contain one or more additional excipients. Suitable excipients include without limitation solubilizers (e.g., C₂-C₈ alcohols), moisturizers or humectants (e.g., glycerol [glycerin], propylene glycol, amino acids and derivatives thereof, polyamino acids and derivatives thereof, and pyrrolidone carboxylic acids and salts and derivatives thereof), surfactants (e.g., sodium laureth sulfate and sorbitan monolaurate), emulsifiers (e.g., cetyl alcohol and stearyl alcohol), thickeners (e.g., methyl cellulose, ethyl cellulose, hydroxymethyl cellulose, hydroxypropyl cellulose, polyvinylpyrrolidone, polyvinyl alcohol and acrylic polymers), and formulation bases or carriers (e.g., polyethylene glycol as an ointment base). As a non-limiting example, the base or carrier of the composition can contain ethanol, propylene glycol and polyethylene glycol (e.g., PEG 300), and optionally an aqueous liquid (e.g., isotonic phosphate-buffered saline).

The topical composition can have any suitable dosage form, such as a spray (e.g., nasal or dermal spray), a solution (e.g., nose drop), a suspension, an emulsion, a cream, a lotion, a gel, an ointment, a paste, a jelly or a foam. In some embodiments, the composition is applied to the mucosa or skin covering a surface area of about 10-800 cm², 10-400 cm² or 10-200 cm². The composition can deliver the therapeutic agent(s) to the mucosa or skin or the underlying tissue. The composition can also be formulated for transmucosal or transdermal administration of the therapeutic agent(s) to the systemic circulation, e.g., as a nasal spray or a nose drop, or as a transmucosal or transdermal patch.

II. Topical Compositions Comprising a Permeation Enhancer and a Volatile Liquid

In further embodiments, a topical composition comprises an NK-1 antagonist (e.g., serlopitant, MK-0303 or MK-8478), a permeation enhancer and a volatile liquid. The composition can optionally contain an additional therapeutic agent. In certain embodiments, the composition contains the NK-1 antagonist (e.g., serlopitant, MK-0303 or MK-8478) in free base form.

The permeation enhancer increases the permeability of the mucosa or skin to the therapeutic agent(s). In some embodiments, the permeation enhancer is selected from C₈-C₁₈ alkyl aminobenzoates (e.g., C₈-C₁₈ alkyl p-aminobenzoates), C₈-C₁₈ alkyl dimethylaminobenzoates (e.g., C₈-C₁₈ alkyl p-dimethylaminobenzoates), C₈-C₁₈ alkyl cinnamates, C₈-C₁₈ alkyl methoxycinnamates (e.g., C₈-C₁₈ alkyl p-methoxycinnamates), and C₈-C₁₈ alkyl salicylates. In certain embodiments, the permeation enhancer is octyl salicylate, octyl p-dimethylaminobenzoate or octyl p-methoxycinnamate, or a combination thereof.

The volatile liquid can be any volatile, mucosa- or skin-tolerant solvent. In certain embodiments, the volatile liquid is a C₂-C₅ alcohol or an aqueous solution thereof, such as ethanol or isopropanol or an aqueous solution thereof. An aerosol propellant (e.g., dimethyl ether) can be considered as a volatile liquid. In some embodiments, the volatile liquid functions as a carrier or vehicle of the composition.

The composition can optionally contain a thickening agent. Non-limiting examples of thickening agents include cellulosic thickening agents (e.g., ethyl cellulose, hydroxypropyl cellulose and hydroxypropyl methylcellulose), povidone, polyacrylic acids/polyacrylates (e.g., Carbopol® polymers), Sepigel® (polyacrylamide/isoparaffin/laureth-7), and the Gantrez® series of polymethyl vinyl ether/maleic anhydride copolymers (e.g., butyl ester of PMV/MA copolymer Gantrez® A-425).

In some embodiments, the composition contains on a weight basis about 0.5-10%, 0.5-5% or 1-5% of the NK-1 antagonist (e.g., serlopitant, MK-0303 or MK-8478), about 1-20%, 1-15% or 1-10% of the permeation enhancer, and about 40-98%, 45-95%, 50-90% or 60-80% of the volatile liquid. In further embodiments, the composition optionally contains on a weight basis about 1-40%, 1-30%, 1-20% or 5-20% water or/and about 0.1-15%, 0.5-10% or 1-5% of a thickening agent.

For purposes of illustration, in certain embodiments a topical spray composition contains about 0.5-5% w/v of the NK-1 antagonist (e.g., serlopitant, MK-0303 or MK-8478), about 2-10% w/v of octyl salicylate or octyl p-methyoxycinnamate, and about 95% aqueous ethanol as the carrier. In further embodiments, a topic gel composition comprises about 0.5-5% w/v of the NK-1 antagonist (e.g., serlopitant, MK-0303 or MK-8478), about 1-10% w/v of octyl salicylate or octyl p-methyoxycinnamate, about 0.5-5% w/v of a Carbopol® polyacrylic acid, and about 70% aqueous ethanol as the carrier, and optionally about 1-10% w/v of a basic solution (e.g., 0.1 N NaOH). In additional embodiments, a topical lotion composition contains about 0.5-5% w/v of the NK-1 antagonist (e.g., serlopitant, MK-0303 or MK-8478), about 1-10% w/v of octyl salicylate or octyl p-methyoxycinnamate, about 1-5% w/v of ethyl cellulose or hydroxypropyl cellulose, and about 90% aqueous ethanol as the carrier.

The composition can further comprise other excipients, such as a compounding agent (e.g., paraffin oil, silicone oil, a vegetable oil, or a fatty ester such as isopropyl myristate), a diluent, a co-solvent (e.g., acetone or a glycol ether such as diethylene glycol monoethyl ether), an emulsifier, a surfactant (e.g., an ethoxylated fatty alcohol, glycerol mono stearate or a phosphate ester), a stabiliser, an antioxidant or a preservative (e.g., a hydroxybenzoate ester), or a combination thereof. For example, a co-solvent or/and a surfactant can be used to maintain the therapeutic agent(s) in solution or suspension at the desired concentration.

The topical composition can have any suitable dosage form, such as a spray (e.g., nasal or dermal spray) or aerosol, a cream, a lotion, a gel, an ointment, a mousse, or any transmucosal or transdermal device (e.g., a patch) that administers a drug by absorption through the mucosa or skin. In some embodiments, the topical composition is applied to the mucosa or skin covering a surface area of about 10-800 cm², 10-400 cm² or 10-200 cm².

III. Topical Compositions Comprising a Permeation Enhancer and Another Excipient

In yet further embodiments, a topical composition comprises an NK-1 antagonist (e.g., serlopitant, MK-0303 or MK-8478), a permeation enhancer, and at least one of a lipophilic solvent, a formulation base and a thickener. In some embodiments, the composition contains a lipophilic solvent and a formulation base, or the same substance can function as both a lipophilic solvent and a formulation base. In further embodiments, the composition contains a lipophilic solvent, a formulation base and a thickener. The composition can optionally comprise an additional therapeutic agent. In certain embodiments, the composition contains the NK-1 antagonist (e.g., serlopitant, MK-0303 or MK-8478) in free base form.

The permeation enhancer increases the permeability of the mucosa or skin to the therapeutic agent(s). Non-limiting examples of permeation enhancers include dimethyl sulfoxide (DMSO), decylmethylsulfoxide, laurocapram, pyrrolidones (e.g., 2-pyrrolidone and N-methyl-2-pyrrolidine), surfactants, alcohols (e.g., oleyl alcohol), polyethylene glycol (e.g., PEG 400), diethylene glycol monoethyl ether, oleic acid, and fatty acid esters (e.g., isopropyl myristate, methyl laurate, glycerol monooleate, and propylene glycol monooleate).

Non-limiting examples of liphophilic solvents include lipophilic alcohols (e.g., hexylene glycol, octyldodecanol, oleyl alcohol and stearyl alcohol), polyethylene glycol (e.g., PEG 100, PEG 300, PEG 400 and PEG 3350), diethylene glycol monoethyl ether, polysorbates (e.g., Tween® 20 to 80), Labrasol®, fatty acid esters (e.g., isopropyl myristate and diisopropyl adipate), diethyl sebacate, propylene glycol monocaprylate, propylene glycol laurate, mono- and di-glycerides (e.g., Capmul® MCM), medium-chain triglycerides, caprylic/capric triglyceride, glyceryl monocaprylate, glyceryl mono-oleate, glyceryl mono-linoleate, glycerol oleate/propylene glycol, mineral oil, and vegetable oils.

A liphophilic solvent may also function as a formulation base or carrier. For example, polyethylene glycol (e.g., from PEG 100 to PEG 3500, such as PEG 300, PEG 400 and PEG 3350) can function as a liphophilic solvent and a formulation base.

The composition can also contain a hydrophilic solvent, such as a C₁-C₅ alcohol (e.g., ethanol, isopropanol, glycerol, propylene glycol and 1,2-pentanediol) or/and water.

The composition can contain a thickener to increase the viscosity or/and the physical stability of the composition. Examples of thickeners include without limitation glycerol, stearyl alcohol, and polymers (e.g., polydimethylsiloxane [dimethicone] and Carbopol® polymers).

In some embodiments, the composition further contains an antioxidant. Non-limiting examples of antioxidants include butylated hydroxyanisole (BHA), butylated hydroxytoluene (BHT), tocopherols (e.g., Vitamin E and esters thereof), flavinoids, glutathione, ascorbic acid and esters thereof, DMSO, and chelating agents (e.g., EDTA and citric acid).

In certain embodiments, the topical composition comprises on a w/w basis about 0.5-10% or 1-5% of the NK-1 antagonist (e.g., serlopitant, MK-0303 or MK-8478), about 2-30% or 5-20% of a permeation enhancer, about 20-80% or 30-70% of a lipophilic solvent that may also function as a formulation base, about 0.1-10% or 1-7.5% of a thickener, and about 0.01-2% or 0.05-1% of an antioxidant. As a non-limiting example, a topical composition can contain the NK-1 antagonist (e.g., serlopitant, MK-0303 or MK-8478), PEG 400 or/and PEG 3350 as lipophilic solvent(s) and formulation base(s), diethylene glycol monoethyl ether, oleyl alcohol or/and isopropyl myristate as permeation enhancer(s), stearyl alcohol as a thickener, and BHT as an antioxidant.

The topical composition can have any suitable dosage form, such as a cream, a lotion, a gel, an ointment, a jelly, a paste, or any transmucosal or transdermal device (e.g., a patch) that administers a drug by absorption through the mucosa or skin.

IV. Topical Compositions Comprising a Permeation Enhancer and an Adhesive

In additional embodiments, a topical composition comprises an NK-1 antagonist (e.g., serlopitant, MK-0303 or MK-8478), a permeation enhancer and an adhesive. The composition can optionally contain an additional therapeutic agent. In certain embodiments, the composition contains the NK-1 antagonist (e.g., serlopitant, MK-0303 or MK-8478) in free base form.

The permeation enhancer increases the permeability of the mucosa or skin to the therapeutic agent(s). The permeation enhancer can be, e.g., a fatty acid ester having a fatty acyl chain length of C₈-C₂₀ or C₁₂-C₁₃ and a C₁-C₆ or C₂-C₄ alcohol component (e.g., isopropanol). In certain embodiments, the permeation enhancer is isopropyl myristate or isopropyl palmitate. In some embodiments, the permeation enhancer is in an amount of about 0.1-20%, 0.5-15%, 1-15%, 2-12% or 4-10% by weight of the composition or the mucosa- or skin-contacting layer of a transmucosal or transdermal patch.

The adhesive maintains contact of the topical composition to the mucosa or skin. Non-limiting examples of adhesives include acrylics/acrylates (e.g., polyacrylates, including polyalkyl acrylates and Duro-Tak® polyacrylates), polyvinyl acetate, ethylenevinylacetate copolymers, polysiloxanes, polyurethanes, plasticized polyether block amide copolymers, natural and synthetic rubbers, plasticized styrene-butadiene rubber block copolymers (e.g., Duro-Tak® 87-6173), and mixtures thereof.

The topical composition can comprise one or more additional excipients. The additional excipient(s) can be, e.g., a diluent, an emollient, a plasticizer, or an agent that reduces irritation to the skin or mucosa, or a combination thereof.

In certain embodiments, the topical composition prior to application to the mucosa or skin is substantially free of water, tetraglycol (glycofurol) or/and a hydrophilic organic solvent (e.g., a C₁-C₅ alcohol).

The composition can administer the therapeutic agent(s) transmucosally or transdermally through a body surface or membrane such as intact unbroken mucosal tissue or intact unbroken skin into the systemic circulation.

In some embodiments, the topical composition is in the form of a transmucosal or transdermal patch for application to the mucosa or skin. The patch has a mucosa- or skin-contacting layer (“skin-contacting layer” here for simplicity) laminated or otherwise attached to a support layer. The skin-contacting layer can be covered by a removable release liner before use to protect the skin-contacting surface and to keep it clean until it is applied to the mucosa or skin.

The support layer of the patch acts as a support for the skin-contacting layer and as a barrier that prevents loss of the therapeutic agent(s) in the skin-contacting layer to the environment. The material of the support layer is compatible with the therapeutic agent(s), the permeation enhancer and the adhesive, and is minimally permeable to the components of the patch. The support layer can be opaque to protect the components of the patch from degradation via exposure to ultraviolet light. The support layer is also capable of binding to and supporting the adhesive layer, yet is sufficiently pliable to accommodate the movements of the subject using the patch. The material of the support layer can be, e.g., a metal foil, a metalized polyfoil, or a composite foil or film containing a polymer (e.g., a polyester [such as polyester terephthalate] or aluminized polyester, polyethylene, polypropylene, polytetrafluoroethylene, a polyethylene methyl methacrylate block copolymer, a polyether block amide copolymer, a polyurethane, polyvinylidene chloride, nylon, a silicone elastomer, rubber-based polyisobutylene, styrene, or a styrene-butadiene or styrene-isoprene copolymer). The release liner can be made of the same material as the support layer, or can be a film coated with an appropriate release surface.

Combination Therapies with a Neurokinin Antagonist and Other Antitussive Agents

Alternative to or in addition to a selective NK-1 antagonist (e.g., serlopitant, MK-0303 or MK-8478), a selective NK-2 antagonist, a selective NK-3 antagonist, or a mixed NK antagonist (e.g., a dual NK-1/NK-2 antagonist, a dual NK-1/NK-3 antagonist, a dual NK-2/NK-3 antagonist or a triple NK-1/NK-2/NK-3 antagonist), or any combination thereof, can be used to treat cough (including acute, subacute and chronic cough) and urge to cough. One or more additional antitussive agents can optionally be used in combination with an NK-1 antagonist (e.g., serlopitant, MK-0303 or MK-8478), an NK-2 antagonist, an NK-3 antagonist or a mixed NK antagonist, or any combination thereof, to treat cough (including acute, subacute and chronic cough) and urge to cough.

Examples of selective and mixed neurokinin receptor antagonists include without limitation:

selective NK-1 antagonists—described elsewhere herein;

selective NK-2 antagonists—ibodutant (MEN-15596), nepadutant (MEN-11420), saredutant (SR-48968), GR-159897, MEN-10376, MEN-10627, NK-5807, ZD-7944, compounds of Formula I disclosed in U.S. Pat. No. 5,789,422 (e.g., Examples 12 and 16), compounds of Formula (I) disclosed in U.S. Pat. No. 5,770,590 (e.g., Examples 22, 27 and 33), and compounds of Formula I disclosed in WO 96/34857 A1 (e.g., Example 22AL);

selective NK-3 antagonists—osanetant (SR-142801), talnetant (SB-223412), SB-218795, SB-222200, SB-235375, and compounds of Formula I characterized as NK-3 antagonists in US 2005/0256164;

dual NK-1/NK-2 antagonists—DNK-333, FK-224, MDL-105172, MDL-105172A, MDL-105212, MDL-105212A, ZD-6021, compounds of Formula I disclosed in U.S. Pat. No. 7,592,344, compounds of Formula (I) disclosed in U.S. Pat. No. 7,402,581, compounds of Formula (1) disclosed in U.S. Pat. No. 6,316,445, compounds of Formula (1) disclosed in U.S. Pat. No. 5,977,139, compounds of Formula I disclosed in U.S. Pat. No. 5,789,422 (e.g., Example 2), compounds of Formula I disclosed in U.S. Pat. No. 5,691,362 (e.g., Examples 2, 7b, 7c, 7d, 12c and 14b), compounds of Formula I disclosed in U.S. Pat. No. 5,688,960 (e.g., Examples 4D and 7B), compounds of Formula (I) disclosed in WO 2005/000845 A2, compounds of Formula I disclosed in WO 00/39114 A2 (e.g., Example 5B), compounds of Formula (1) disclosed in WO 98/27086 A1 (e.g., Compounds A and C), and compounds of Formula I disclosed in WO 96/34857 A1 (e.g., Examples 22AK, 39F and 42L);

dual NK-1/NK-3 antagonists—compounds of Formula I characterized as potential dual NK-1/NK-3 antagonists in US 2005/0256164;

dual NK-2/NK-3 antagonists—compounds of Formula I disclosed in US 2007/0219214 and compounds of Formula (I) disclosed in WO 02/094821 A1;

triple NK-1/NK-2/NK-3 antagonists—CS-003, SCH-206272, compounds of Formula I disclosed in WO 00/39114 A2 (e.g., Examples 6E, 6K, 6M, 6S, 6Y, 7F, 8A, 10B, 11A, 16 and 18A), and compounds of Formula I disclosed in WO 96/34857 A1 (e.g., Example 1);

and analogs, derivatives, prodrugs, metabolites and salts thereof.

In certain embodiments, the NK-2 antagonist is not, or does not include, saredutant (SR-48968), MEN-10627 or a piperidinyl compound of Formula I disclosed in U.S. Pat. No. 5,789,422. In some embodiments, the NK-3 antagonist is not, or does not include, osanetant (SR-142801), talnetant (SB-223412) or SB-235375. In further embodiments, the dual NK-1/NK-2 antagonist is not, or does not include, DNK-333, FK-224, MDL-105212, MDL-105212A, ZD-6021, a compound of Formula (I) disclosed in U.S. Pat. No. 8,476,253, a compound of Formula (1) disclosed in U.S. Pat. No. 6,316,445, a compound of Formula (1) disclosed in U.S. Pat. No. 5,977,139, a piperidinyl compound of Formula I disclosed in U.S. Pat. No. 5,789,422, an indolyl compound of Formula I disclosed in U.S. Pat. No. 5,691,362, or a compound of Formula (1) disclosed in WO 98/27086 A1. In other embodiments, the triple NK-1/NK-2/NK-3 antagonist is not, or does not include, CS-003 or SCH-206272.

Examples of additional antitussive agents include without limitation opioids (e.g., mu-opioid receptor agonists and kappa-opioid receptor agonists), NOP/ORL-1 receptor agonists, agonists of sigma (e.g., σ₁ or/and σ₂) receptors, NMDAR antagonists, cannabinoid receptor type 2 (CB₂) agonists, butamirate class of antitussives, TRPV1 antagonists, TRPV4 antagonists, TRPA1 antagonists, inhibitors of bradykinin or receptors therefor (e.g., B1 and B2), inhibitors of inflammatory prostaglandins (e.g., PGE2) or receptors therefor (e.g., EP3), inhibitors of calcitonin gene-related peptide (CGRP) or receptor therefor, antagonists of protease-activated receptors (PARs) and inhibitors of activating proteases, anti-inflammatory agents (e.g., antihistamines, mast cell stabilizers, corticosteroids, immunomodulators, non-steroidal anti-inflammatory drugs, leukotriene receptor antagonists and 5-lipoxygenase inhibitors), antagonists of P2X purinergic receptors (e.g., P2X3 and P2X2/3 receptor antagonists), decongestants, beta (e.g., β₂) adrenergic receptor agonists, antagonists of muscarinic acetylcholine receptors (e.g., M1, M2, M3, M4 or/and M5), inhibitors of gastrin-releasing peptide (GRP) or the receptor therefor (GRPR or BBR2), antipyretics, anticonvulsants, GABA-B receptor agonists, antidepressants, 5-HT_{1A} agonists, inhibitors of nerve growth factor (NGF) or receptors therefor (e.g., TrkA and LNGFR), inhibitors of brain-derived neurotrophic factor (BDNF) or receptors thereof (e.g., TrkB and LNGFR), α7 nicotinic acetylcholine receptor agonists, Fritillaria alkaloids, peripheral antitussives (e.g., dropropizine [dipropizine], levodropropizine, moguisteine [inhibitor of rapidly adapting receptors], and naringin [inhibitor of substance P content and NK-1 expression]), local anesthetics, vitamins (e.g., vitamin C), minerals (e.g., zinc), sweet substances (e.g., honey and sugar syrup), and therapeutic agents that treat the underlying cause of the cough or urge to cough, including but not limited to antihistamines for putative post-nasal drips; corticosteroids (e.g., prednisone) or/and bronchodilators (e.g., β₂-adrenoreceptor agonists) for putative asthma; leukotriene receptor antagonists or/and mast cell stabilizers (e.g., cromoglicic acid and nedocromil) for putative asthma; corticosteroids for putative NAEB; corticosteroids or/and bronchodilators (e.g., β₂-adrenoreceptor agonists) for putative COPD; first-generation antihistamines with anticholinergic activity, bronchodilators (e.g., ipratropium bromide) or/and decongestants (e.g., oxymetazoline hydrochloride) for putative UACS; decongestants (e.g., pseudoephedrine) or/and antibiotics for putative bacterial sinusitis; antibiotics for putative bacterial bronchitis, pertussis or tuberculosis; proton-pump inhibitors or/and prokinetic agents for putative GERD; and anticonvulsants or/and tricyclic antidepressants for neurogenic cough.

Opioid receptors (including mu, kappa and delta) are present on sensory nerves innervating the airways as well as on neurons in the cough center in the brainstem (Belvisi 2009 and Bolser 2009). Therefore, opioids can be used as antitussives. Non-limiting examples of opioids include alfentanil, apomorphine, buprenorphine, codeine, desomorphine, dextromethorphan, dextromoramide, dextropropoxyphene, diamorphine, dihydrocodeine, dihydrocodeinone enol acetate, dihydromorphine, ethylmorphine, fentanyl, hydrocodone (dihydrocodeinone), hydromorphone, methadone, morphine, oxycodone, oxymorphone, papvereturn, papverine, pethidine, pholcodine, tapentadol, tramadol, the benzomorphan class of opioids (including the D-stereoisomer) that act on opioid or/and sigma receptors (e.g., alazocine, anazocine, bremazocine, butinazocine, carbazocine, cogazocine, cyclazocine, dezocine, eptazocine, etazocine, ethylketocyclazocine, fluorophen, gemazocine, ibazocine, ketazocine, metazocine, moxazocine, pentazocine, phenazocine, quadazocine, thiazocine, tonazocine, volazocine, zenazocine, 8-CAC and 5,9-DEHB), peptide opioids (e.g., enkephalins such as DAMGO), and analogs, derivatives and salts thereof. In certain embodiments, the opioid is or includes dextromethorphan, codeine, dihydrocodeine or hydrocodone.

Activation of the mu-opioid receptor or the kappa-opioid receptor depresses the cough reflex (Kamei 1996). Examples of mu-opioid receptor agonists include without limitation 8-carboxamidocyclazocine (8-CAC), codeine, [D-Ala², N-MePhe⁴, Gly⁵-ol]-enkephalin (DAMGO), eluxadoline, hydrocodone, levomethorphan, levorphanol, morphine, TRIMU 5, and analogs, derivatives and salts thereof. In certain embodiments, the μ-opioid receptor agonist is or includes codeine or DAMGO. Non-limiting examples of kappa-opioid receptor agonists include asimadoline, bremazocine, butorphan, 8-carboxamidocyclazocine, cyclorphan, difelikefalin (CR845), dynorphins (e.g., dynorphin A), eluxadoline, enadoline, erinacine E, ketazocine, levomethorphan, levorphanol, nalfurafine (TRK-820), salvinorin A, 2-methoxymethyl salvinorin B, 2-ethoxymethyl salvinorin B, 2-fluoroethoxymethyl salvinorin B, spiradoline, tifluadom, BRL-52537, FE 200665, GR-89696, HZ-2, ICI-199,441, ICI-204,448, LPK-26, SA-14867, U-50488, U-50488H, U-69593, 2-phenylbenzothiazoline-type compounds, and analogs, derivatives and salts thereof. In certain embodiments, the κ-opioid receptor agonist is or includes U-50488 or U-50488H.

Delta-opioid receptor agonists and agonists of the nociceptin opioid peptide (NOP)/opioid receptor-like 1 (ORL-1) receptor also inhibit cough (Bolser 2009). Examples of delta-opioid receptor agonists include without limitation, Leu-enkephalin, Met-enkephalin, [D-Ala², D-Leu⁵] enkephalin (DADLE), [D-Pen², D-Pen⁵]-enkephalin (DPDPE), deltorphins (e.g., deltorphin II), mitragynine, mitragynine pseudoindoxyl, N-phenethyl-14-ethoxymetopon, 7-spiroindanyloxymorphone, xorphanol, cannabidiol, tetrahydrocannabinol, ADL-5747, ADL-5859, AR-M100390, BU-48, BW373U86, DPI-221, DPI-287, DPI-3290, JNJ-20788560, NIH-11082, RWJ-394674, SB-235863, SNC-80, TAN-67, and analogs, derivatives and salts thereof. Non-limiting examples of NOP/ORL-1 receptor agonists include nociceptin, buprenorphine (partial agonist of NOP, δ-opioid and μ-opioid receptors), norbuprenorphine (full agonist of NOP, δ-opioid and μ-opioid receptors, and partial agonist of κ-opioid receptor; peripherally selective), cebranopadol (full agonist of NOP, δ-opioid and μ-opioid receptors, and partial agonist of κ-opioid receptor), etorphine, MCOPPB, BU-08028 (agonist of NOP and μ-opioid receptors), MT-7716, NNC 63-0532, Ro64-6198, Ro65-6570, SCH-221510, SR-8993, SR-16435 (partial agonist of NOP and μ-opioid receptors), TH-030418, and analogs, derivatives and salts thereof.

Sigma receptors (including σ₁ and σ₂) are also found on neurons in the cough center in the brainstem (Bolser 2009). Thus, sigma agonists can act as antitussives. Examples of agonists of sigma (e.g., σ₁ or/and σ₂) receptors include without limitation afobazole (selective for σ₁), allylnormetazocine, arketamine, berberine, buprenorphine, citalopram, dehydroepiandrosterone (DHEA), dehydroepiandrosterone sulfate (DHEA-S), dextrallorphan (selective for σ₁), dextromethorphan (relatively selective for σ₁), dextrorphan, diacetylmorphine, N,N-dimethyltryptamine, dimemorfan, ditolylguanidine, escitalopram, fluoxetine, fluvoxamine, igmesine, ketamine, lamotrigine, memantine (selective for σ₁), methoxetamine (selective for σ₁), methylphenidate, morphine, noscapine, opipramol, pentazocine, pentoxyverine (carbetapentane, selective for σ₁), phencyclidine, 4-PPBP, (+)-3-PPP, pregnenolone, pregnenolone sulfate, quetiapine, siramesine, tapentadol, tramadol, BD 1031 (selective for σ₁), BD-1052 (selective for σ₁), L-687,384 (selective for σ₁), OFC-14523, PB-28 (selective for σ₂), PRE-084 (selective for σ₁), SA-4503 (selective for σ₁), UMB-23, UMB-82, and analogs, derivatives and salts thereof. In certain embodiments, the sigma agonist is or includes noscapine, pentoxyverine or memantine.

Antagonists of the N-methyl-D-aspartate receptor (NMDAR), an excitatory glutamate receptor and cation-channel protein on neurons in the CNS, can also be used as antitussives. NMDAR antagonists reduce neuronal excitability and thus can inhibit synaptic transmission to and from the cough center in the brainstem and thereby inhibit the central cough reflex. NMDAR antagonists can reduce neuronal cough hypersensitivity or cough sensitization, and can potentiate or synergize the antitussive activity of a neurokinin antagonist, such as an NK-1 antagonist (e.g., serlopitant, MK-0303 or MK-8478), or vice versa. In some embodiments, the NMDAR antagonists are uncompetitive antagonists (or channel blockers) that have a moderate affinity (e.g., a Kᵢ or IC₅₀ from about 200 nM to about 10 μM) for the dizocilpine (MK-801)/phencyclidine-binding site at or near the Mg²⁺-binding site in the opened ion channel of activated NMDAR, which allows them to exert antitussive action while preserving physiological NMDAR activity. Examples of such NMDAR antagonists include without limitation alaproclate, amantadine, atomoxetine, budipine, delucemine, dextrallorphan, dextromethorphan, dextrorphan, dexanabinol, eliprodil, ketamine, lanicemine, minocycline, memantine, nitromemantine, NEFA (a tricyclic small molecule), neramexane, orphenadrine, procyclidine, ARL/FPL 12495/12495AA (desglycine metabolite of remacemide), and analogs, derivatives and salts thereof. In some embodiments, the NMDAR antagonist is or includes memantine, nitromemantine, amantadine, lanicemine, neramexane, dextrallorphan, dextromethorphan or procyclidine. In certain embodiments, the NMDAR antagonist is or includes memantine, nitromemantine, dextrallorphan or dextromethorphan. In other embodiments, the NMDAR antagonist is not, or does not include, dextromethorphan or dextrorphan.

In certain embodiments, a neurokinin antagonist, such as an NK-1 antagonist (e.g., serlopitant, MK-0303 or MK-8478), is used in combination with memantine or a derivative thereof (e.g., nitromemantine) to treat cough, including chronic cough. In some embodiments, the daily dose of memantine or a derivative thereof (e.g., nitromemantine) is about 1-30 mg, 1-10 mg, 10-20 mg or 20-30 mg, or as determined by the treating physician, which can be administered in a single dose or divided doses. In certain embodiments, the daily dose of memantine or a derivative thereof (e.g., nitromemantine) is about 1, 5, 10, 15, 20, 25 or 30 mg. In further embodiments, the daily dose of memantine or a derivative thereof (e.g., nitromemantine) is about 1-5 mg, or about 1, 2, 3, 4 or 5 mg. In other embodiments, the daily dose of memantine or a derivative thereof (e.g., nitromemantine) is about 5-10 mg, or about 5, 6, 7, 8, 9 or 10 mg. In additional embodiments, memantine or a derivative thereof (e.g., nitromemantine) is administered in one or more daily initial doses, followed by a daily maintenance dose for the duration of treatment, wherein the daily maintenance dose can be any daily dose described above. The one or more initial doses can be smaller or larger than (e.g., 1.5, 2, 3, 4 or 5 times smaller or larger than) the maintenance dose. In some embodiments, a first initial dose is administered once daily for the first week, a second initial dose 2× larger than the first initial dose is administered once daily for the second week, a third initial dose 3× larger than the first initial dose is administered once daily for the third week, and a maintenance dose 4× larger than the first initial dose is administered once daily for the fourth week and thereafter for the duration of therapy. As a non-limiting example, a first initial dose of about 3 mg of memantine or a derivative thereof (e.g., nitromemantine) can be administered once daily for the first week, a second initial dose of about 6 mg can be administered once daily for the second week, a third initial dose of about 9 mg can be administered once daily for the third week, and a maintenance dose of about 12 mg can be administered once daily for the fourth week and thereafter for the duration of therapy.

Like opioid receptors, cannabinoid receptor type 2 (CB₂) is expressed in the peripheral nervous system as well as on neurons in the cough center in the brainstem (Belvisi 2009 and Bolser 2009). Hence, CB₂ agonists can be utilized as antitussives. Non-limiting examples of CB₂ agonists include anandamide (N-arachidonoylethanolamine), 2-arachidonoylglycerol, virodhamine [O-arachidonoylethanolamine], palmitoylethanolamide (N-palmitoylethanolamine), AM-1241, GW-405833, GW-833972A, HU-308, JWH-015, JWH-133, L-759633, L-759656, S-777469, and analogs, derivatives and salts thereof.

The butamirate class of compounds binds to the cough center (e.g., the dextromethorphan-binding site) in the medulla oblongata and hence can be employed as antitussives. The butamirate class of antitussives includes without limitation butamirate (brospamin), oxeladin, pentoxyverine, and analogs, derivatives and salts thereof.

Stimulation of tachykinin-containing, capsaicin-sensitive jugular vagal afferent unmyelinated C-fibers innervating the airways via activation of vanilloid receptor 1 (VR-1) (also called transient receptor potential vanilloid type 1 [TRPV1]) expressed on the C-fibers causes coughing (Canning, AJPRICP 2006). For instance, inhalation of the TRPV1 agonist capsaicin causes an “itchy” feeling in the airways and evokes cough, and citric acid induces cough through activation of TRPV1. Furthermore, airway mucosas of subjects suffering from chronic cough have five-fold greater levels of TRPV1 than unaffected subjects (LaVinka 2013). Thus, TRPV1 antagonists can be used to inhibit coughing. Non-limiting examples of TRPV1 antagonists include capsazepine, iodo-resiniferatoxin (IRTX), ruthenium red, AMG-517, GRC-6211, JNJ-1720321, NGD-8243, SB-705498, Xen-D0501, specialized pro-resolving mediators (SPMs, e.g., metabolites of polyunsaturated fatty acids [PUFAs] such as lipoxins, resolvins [including resolvins derived from 5Z,8Z,11Z,14Z,17Z-eicosapentaenoic acid {EPA}, resolvins derived from 4Z,7Z,10Z,13Z,16Z,19Z-docosahexaenoic acid {DHA}, and resolvins derived from 7Z,10Z,13Z,16Z,19Z-docosahexaenoic acid {n-3 DPA}], protectins/neuroprotectins [including DHA-derived protectins/neuroprotectins and n-3 DPA-derived protectins/neuroprotectins], maresins [including DHA-derived maresins and n-3 DPA-derived maresins], n-3 DPA metabolites, n-6 DPA {4Z,7Z,10Z,13Z,16Z-docosapentaenoic acid} metabolites, oxo-DHA metabolites, oxo-DPA metabolites, docosahexaenoyl ethanolamide metabolites, cyclopentenone prostaglandins [e.g., Δ12-PGJ2 and 15-deoxy-Δ12,14-PGJ2], and cyclopentenone isoprostanes [e.g., 5,6-epoxyisoprostane A2 and 5,6-epoxyisoprostane E2]), and analogs, derivatives and salts thereof.

Activation of TRPV4 on unmyelinated C-fibers with a TRPV4 agonist also evokes cough (Keller 2017). Hence, TRPV4 antagonists can be used as antitussives. Examples of TRPV4 antagonists include without limitation ruthenium red, HC-067047, RN-1734, RN-9893, SPMs (e.g., PUFA metabolites such as resolvins [e.g., resolvin D1]), and analogs, derivatives and salts thereof.

Likewise, activation of transient receptor potential ankyrin A1 (TRPA1) on jugular vagal sensory unmyelinated C-fibers (which may also express TRPV1) innervating the airways (including the trachea, bronchi and lungs) by a wide range of stimuli such as cinnamaldehyde, irritants in cigarette smoke (e.g., acrolein and crotonaldehyde), air pollutants and oxidative agents stimulates the C-fibers and triggers cough (Birrell 2009). Therefore, TRPA1 antagonists can be used as antitussives. Examples of TRPA1 antagonists include without limitation camphor, isopentenyl pyrophosphate, ruthenium red, A967079, AP-18, GRC-17536, HC-030031, (4R)-1,2,3,4-tetrahydro-4-[3-(3-methoxypropoxy)phenyl]-2-thioxo-5H-indeno[1,2-d]pyrimidin-5-one, 2-amino-4-arylthiazole compounds disclosed in WO 2012/085662 A1, bicyclic heterocyclic compounds of Formula (I) disclosed in WO 2017/064068 A1, SPMs (e.g., metabolites of PUFAs), and analogs, derivatives and salts thereof.

Desensitization of unmyelinated C-fibers by repeated application of capsaicin reduces coughing induced by activation of myelinated Aδ-fibers. Interestingly, desensitization of unmyelinated C-fibers that express both TRPA1 and TRPV1 also reduces TRPA1-initiated coughing (LaVinka 2013). Hence, coughing can be curtailed by TRPV1 agonists that cause decrease in TRPV1 activity (desensitization) upon prolonged or repeated exposure of TRPV1 to the stimuli, including but not limited to capsaicin, camphor, carvacrol, menthol, methyl salicylate, resiniferatoxin (RTX), tinyatoxin, and analogs, derivatives and salts thereof.

Inhalation of bradykinin also causes an “itchy” feeling in the airways and bronchoconstriction and evokes cough. Furthermore, bradykinin sensitizes the cough reflex, enhancing cough response to citric acid. Patients taking an angiotensin-converting enzyme (ACE) inhibitor for hypertension often suffer from chronic cough (ACE degrades bradykinin). Bradykinin depolarizes vagal afferent fibers, thereby activating unmyelinated C-fibers in the jugular ganglion, and bradykinin exerts tussigenic effect via activation of both TRPV1 and TRPA1 on C-fibers (LaVinka 2013). Thus, the cough reflex can be suppressed by blocking the effects of bradykinin. Non-limiting examples of inhibitors of bradykinin or receptors therefor (e.g., B1 and B2) or the production thereof include bradykinin inhibitors (e.g., aloe, bromelain and polyphenols), bradykinin B1 receptor antagonists {e.g., safotibant [LF22-0542] and [Leu⁸]-bradykinin(1-8)}, bradykinin B2 receptor antagonists (e.g., icatibant [HOE-140], FR-173657 and D-Arg-[Hyp³, Thi^{5,8}, D-Phe⁷]-bradykinin), inhibitors of kallikreins (e.g., ecallantide, camostat, nafamostat, gabexate and C1-inhibitor), and analogs, derivatives and salts thereof. In certain embodiments, the inhibitor of bradykinin or a receptor therefor or the production thereof is or includes a B2 antagonist (e.g., icatibant).

Similar to bradykinin, prostaglandin E2 (PGE2) causes cough by depolarizing vagal sensory neurons through indirect activation or sensitization of TRPV1 and TRPA1 on C-fibers following activation of the prostaglandin EP3 receptor (LaVinka 2013). Therefore, inhibition of the effects of inflammatory prostaglandins such as PGE2 can curtail coughing. Examples of inhibitors of inflammatory prostaglandins (e.g., PGE2) or receptors therefor (e.g., EP3) or the production thereof include, but are not limited to, non-steroidal anti-inflammatory drugs (NSAIDs [supra], e.g., non-selective COX-1/COX-2 inhibitors such as aspirin and indomethacin, and selective COX-2 inhibitors such as coxibs), glucocorticoids (supra), cyclopentenone prostaglandins (e.g., prostaglandin J2 [PGJ2], Δ12-PGJ2 and 15-deoxy-Δ12,14-PGJ2), and analogs, derivatives and salts thereof.

Activation of TRPA1 or TRPV1 on vagal airway unmyelinated C-fibers leads to release of inflammatory neuropeptides from the C-fibers, including tachykinins and calcitonin gene-related peptide (CGRP) (LaVinka 2013). The neuropeptides cause neurogenic inflammation, which is involved in, e.g., chronic cough. Therefore, antitussives include inhibitors of CORP or receptor therefor or the production thereof, including but not limited to CORP receptor antagonists (e.g., olcegepant, telcagepant, ubrogepant, eptinezumab [ALD-403], AMG-334, LY-2951742, TEV-48125, and compounds of Formula I disclosed in WO 2007/146349 A2), and analogs, derivatives, fragments and salts thereof.

Protease-activated receptors (PARs) and proteases activating them are involved in airway inflammation and cough sensitivity. The trypsin-like protease thrombin activates vagal bronchopulmonary C-fibers by activating PAR1. PAR2 is also implicated in airway inflammation and hyperactivity to inhaled stimulants, and PAR2 agonists cause bronchoconstriction. Activation of PAR2 by tryptase released from mast cells leads to release of PGE2 in the airways, and PGE2 induces cough and sensitizes the pulmonary C-fiber cough reflex. In addition, PAR2 potentiates cough by sensitizing TRPV1 to cough evocation (LaVinka 2013). Hence, PAR antagonists and protease inhibitors can be utilized as antitussives. Non-limiting examples of antagonists of protease-activated receptors (PARs) and inhibitors of activating proteases include PAR1 antagonists (e.g., SCH-530,348), PAR2 antagonists {e.g., AY-117, ENMD-1068, ENMD-106836, GB-83, tetracyclines (e.g., doxycycline, minocycline and tetracycline), FSLLRY-NH₂ (PAR-3888-PI), Ac-FSLLRY-NH₂ and anti-PAR2 antibodies (e.g., SAM-11 [SC-13504], P2pal-21 and P2pal-2135}, PAR4 antagonists {e.g, ethanol, YD-3, statins (e.g., atorvastatin, cerivastatin, fluvastatin, lovastatin, pitavastatin, pravastatin, rosuvastatin and simvastatin), pepducin P4 pal-10, pepducin P4 pal-15, trans-cinnamoyl-APGKF-NH₂, trans-cinnamoyl-YPGKF-NH₂, and anti-PAR4 antibodies (e.g., C-19 and SC-1249)}, inhibitors of serine proteases {e.g., benzamidine hydrochloride, 4-iodo-1-benzothiophene-2-carboximidamide hydrochloride (inhibits trypsin and tryptase), inhibitors of kallikreins (e.g., camostat, nafamostat, gabexate, ecallantide and C1-inhibitor), trypsin inhibitors (e.g., tosyllysine chloromethyl ketone [TLCK] hydrochloride, α₁-antitrypsin, aprotinin, ovomucin and soybean trypsin inhibitor), and tryptase inhibitors (e.g., camostat, nafamostat, gabexate, AMG-126737 and APC-366)}, inhibitors of cysteine proteases {e.g., E-64 (non-specific inhibitor), JNJ-10329670, RWJ-445380, cystatin C, leupeptin, stefin A, stefin B, testican-1, chloroquine, fluoromethyl ketone, naphthalene endoperoxide (inhibits cathepsin B, L and S), CA-074 (inhibits cathepsin B), odanacatib (MK-0822, inhibits cathepsin K), CLIK-148 and CLIK-195 (inhibit cathepsin L), and CLIK-60 and E-6438 (inhibit cathepsin S)}, and analogs, derivatives, fragments and salts thereof.

Histamine increases cough sensitivity by sensitizing vagal bronchopulmonary fibers to tussigenic agents such as capsaicin, citric acid and mechanical stimulation. Increased cough sensitivity can lead to chronic cough, and chronic cough sufferers have elevated levels of histamine in their sputum and lungs (LaVinka 2013). Antihistamines can be used to curtail the effects of histamine such as inflammation, increased microvascular permeability (which results in a runny nose), and increased cough sensitivity. Antihistamines that can be used to treat, e.g., cough (e.g., chronic cough) or a cough-associated respiratory condition (e.g., eosinophilic bronchitis) include, but are not limited to, antihistamines that inhibit action at the histamine H₁ or H₄ receptor. In certain embodiments, an H₁ antihistamine (e.g., a first-generation H₁ antihistamine) is used to treat post-nasal drip (aka PNDS or UACS) or cough associated therewith. Non-limiting examples of H₁ antihistamines include acrivastine, antazoline, astemizole, azatadine, azelastine, bepotastine, bilastine, bromodiphenhydramine, brompheniramine, buclizine, carbinoxamine, cetirizine, chlorcyclizine, chlorodiphenhydramine, chlorpheniramine, chlorpromazine, chloropyramine, cidoxepin, clemastine, cyclizine, cyproheptadine, desloratadine, dexbrompheniramine, dexchlorpheniramine, dimenhydrinate, dimetindene, diphenhydramine, doxepin, doxylamine, ebastine, embramine, esmirtazapine [(S)-(+)-mirtazapine], fexofenadine, hydroxyzine, ketotifen, levocabastine, levocetirizine, loratadine, meclozine (meclizine), mepyramine, mirtazapine, mizolastine, olopatadine, orphenadrine, phenindamine, pheniramine, phenyltoloxamine, promethazine, pyrilamine, quetiapine, quifenadine, rupatadine, terfenadine, trimeprazine (alimemazine), tripelennamine, triprolidine, and analogs, derivatives and salts thereof. Examples of H₄ antihistamines include without limitation clobenpropit, thioperamide, A943931, A987306, JNJ-7777120, VUF-6002, ZPL-389, and analogs, derivatives and salts thereof. In certain embodiments, the H₁ antihistamine is or includes diphenhydramine or chlorpheniramine. In further embodiments, the H₄ antihistamine is or includes JNJ-7777120.

If desired (e.g., for relief from coughing during the day), a non-sedating antitussive agent can be used. For example, second-generation and third-generation H₁ antihistamines are designed to be non-sedating, or less sedating than first-generation H₁ antihistamines, through reduced crossing of the blood-brain barrier and increased action at peripheral histamine H₁ receptors. Non-limiting examples of second-generation and third-generation H₁ antihistamines include acrivastine, astemizole, azelastine, bepotastine, bilastine, cetirizine, cidoxepin, levocetirizine, ebastine, fexofenadine, levocabastine, loratadine, desloratadine, mizolastine, olopatadine, quifenadine, rupatadine, terfenadine, and analogs, derivatives and salts thereof.

A sedating antitussive agent can also be used, such as at night for relief from coughing during nighttime. For instance, sedating first-generation H₁ antihistamines that cross the blood-brain barrier can be taken at night to aid with sleep and to reduce nighttime coughing. Non-limiting examples of first-generation H₁ antihistamines include antazoline, azatadine, brompheniramine, buclizine, bromodiphenhydramine (bromazine), carbinoxamine, chlorcyclizine, chloropyramine, chlorpromazine, cyclizine, chlorpheniramine, chlorodiphenhydramine, clemastine, cyproheptadine, dexbrompheniramine, dexchlorpheniramine, dimenhydrinate, dimetindene, diphenhydramine, doxepin, doxylamine, embramine, esmirtazapine, hydroxyzine, ketotifen, meclozine (meclizine), mepyramine, mirtazapine, orphenadrine, phenindamine, pheniramine, phenyltoloxamine, promethazine, pyrilamine, quetiapine, trimeprazine, tripelennamine, triprolidine, and analogs, derivatives and salts thereof.

Bronchoalveolar lavage of subjects with chronic non-productive cough shows elevated numbers of mast cells and inflammatory cells and airway inflammation. Upon activation, mast cells degranulate and release tussigenic mediators such as histamine and proteases (e.g., tryptase) (LaVinka 2013). Mast cell stabilizers block a calcium channel essential for mast cell degranulation, stabilizing the mast cell and thereby preventing the release of histamine and related inflammatory mediators and other kinds of tussigenic mediators. Therefore, a mast cell stabilizer can be used to treat cough (e.g., chronic cough) or a cough-associated medical condition (e.g., an inflammatory respiratory condition [e.g., asthma] or a lung tissue disorder [e.g., pulmonary fibrosis such as IPF]). Examples of mast cell stabilizers include without limitation β₂-adrenergic agonists (supra), cromoglicic acid (cromolyn), ketotifen, methylxanthines, nedocromil, olopatadine, omalizumab, pemirolast, quercetin, and analogs, derivatives and salts thereof. In certain embodiments, the mast cell stabilizer is or includes nedocromil or cromoglicic acid or a salt thereof (e.g., cromolyn sodium).

Airway inflammation can cause cough (including unproductive cough and chronic cough) or enhance cough reflex sensitivity. For instance, airway inflammation can reduce the activation threshold of cough-evoking vagal sensory fibers, thereby increasing their sensitivity to airway stimuli. In animal models, many exogenous and endogenous inflammatory mediators can acutely induce airway sensitization, including prostanoids, leukotrienes, lipid mediators, kinins, neurotrophins, acids, oxidants and ATP, which can come from infiltrating or resident immune cells or injured airway epithelium (Keller 2017). Therefore, anti-inflammatory agents in general can be useful as antitussives.

The glucocorticoid class of corticosteroids has anti-inflammatory and vasoconstrictive effects and thus can be utilized to treat cough or cough-associated conditions. In some embodiments, a corticosteroid is administered (e.g., by oral or nasal inhalation) to treat an airway inflammatory disease (e.g., asthma, allergic rhinitis, COPD or NAEB) or cough associated therewith. Non-limiting examples of corticosteroids (including glucocorticoids) include hydrocortisone types (e.g., cortisone and derivatives thereof [e.g., cortisone acetate], hydrocortisone and derivatives thereof [e.g., hydrocortisone acetate, hydrocortisone-17-aceponate, hydrocortisone-17-buteprate, hydrocortisone-17-butyrate and hydrocortisone-17-valerate], prednisolone, methylprednisolone and derivatives thereof [e.g., methylprednisolone aceponate], prednisone, and tixocortol and derivatives thereof [e.g., tixocortol pivalate]), betamethasone types (e.g., betamethasone and derivatives thereof [e.g., betamethasone dipropionate, betamethasone sodium phosphate and betamethasone valerate], dexamethasone and derivatives thereof [e.g., dexamethasone sodium phosphate], and fluocortolone and derivatives thereof [e.g., fluocortolone caproate and fluocortolone pivalate]), halogenated steroids (e.g., alclometasone and derivatives thereof [e.g., alclometasone dipropionate], beclometasone and derivatives thereof [e.g., beclometasone dipropionate], clobetasol and derivatives thereof [e.g., clobetasol-17-propionate], clobetasone and derivatives thereof [e.g., clobetasone-17-butyrate], desoximetasone and derivatives thereof [e.g., desoximetasone acetate], diflorasone and derivatives thereof [e.g., diflorasone diacetate], diflucortolone and derivatives thereof [e.g., diflucortolone valerate], fluprednidene and derivatives thereof [e.g., fluprednidene acetate], fluticasone and derivatives thereof [e.g., fluticasone propionate], halobetasol [ulobetasol] and derivatives thereof [e.g., halobetasol proprionate], halometasone and derivatives thereof [e.g., halometasone acetate], and mometasone and derivatives thereof [e.g., mometasone furoate]), acetonides and related substances (e.g., amcinonide, budesonide, ciclesonide, desonide, fluocinonide, fluocinolone acetonide, flurandrenolide [flurandrenolone or fludroxycortide], halcinonide, triamcinolone acetonide and triamcinolone alcohol), carbonates (e.g., prednicarbate), and analogs, derivatives and salts thereof. In certain embodiments, the corticosteroid is or includes prednisone or beclometasone or a derivative thereof (e.g., beclometasone dipropionate). In further embodiments, ciclesonide or beclometasone or a derivative thereof (e.g., beclometasone dipropionate) is administered (e.g., by oral or nasal inhalation) to ameliorate symptoms of airway inflammatory diseases, such as asthma, allergic rhinitis and COPD.

Another class of anti-inflammatory agents is immunomodulators. Non-limiting examples of immunomodulators include imides (e.g., thalidomide, lenalidomide, pomalidomide and apremilast), xanthine derivatives (e.g., lisofylline, pentoxifylline and propentofylline), and analogs, derivatives and salts thereof. Immunomodulators may suppress unproductive (dry) cough through, e.g., their anti-inflammatory action or inhibition of pulmonary sensory nerve fibers. In some embodiments, an immunomodulator is used to treat cough (e.g., chronic cough or intractable cough) associated with interstitial lung disease or pulmonary fibrosis (e.g., IPF). In certain embodiments, the immunomodulator is or includes thalidomide. Other anti-inflammatory agents that can be used to treat cough (e.g., chronic cough or intractable cough) associated with interstitial lung disease or pulmonary fibrosis (e.g., IPF) include without limitation pirfenidone and nintedanib, both of which also have antifibrotic activity.

Examples of non-steroidal anti-inflammatory drugs (NSAIDs) include without limitation:

acetic acid derivatives, such as aceclofenac, bromfenac, diclofenac, etodolac, indomethacin, ketorolac, nabumetone, sulindac, sulindac sulfide, sulindac sulfone and tolmetin;

anthranilic acid derivatives (fenamates), such as flufenamic acid, meclofenamic acid, mefenamic acid and tolfenamic acid;

enolic acid derivatives (oxicams), such as droxicam, isoxicam, lornoxicam, meloxicam, piroxicam and tenoxicam;

propionic acid derivatives, such as fenoprofen, flurbiprofen, ibuprofen, dexibuprofen, ketoprofen, dexketoprofen, loxoprofen, naproxen and oxaprozin;

salicylates, such as diflunisal, salicylic acid, acetylsalicylic acid (aspirin), choline magnesium trisalicylate, and salsalate;

COX-2-selective inhibitors, such as apricoxib, celecoxib, etoricoxib, firocoxib, fluorocoxibs (e.g., fluorocoxibs A-C), lumiracoxib, mavacoxib, parecoxib, rofecoxib, tilmacoxib (JTE-522), valdecoxib, 4-O-methylhonokiol, niflumic acid, DuP-697, CG100649, GW406381, NS-398, SC-58125, benzothieno[3,2-d]pyrimidin-4-one sulfonamide thio-derivatives, and COX-2 inhibitors derived from Tribulus terrestris;

other kinds of NSAIDs, such as monoterpenoids (e.g., eucalyptol and phenols [e.g., carvacrol]), anilinopyridinecarboxylic acids (e.g., clonixin), sulfonanilides (e.g., nimesulide), and dual inhibitors of lipooxygenase (e.g., 5-LOX) and cyclooxygenase (e.g., COX-2) (e.g., chebulagic acid, licofelone, 2-(3,4,5-trimethoxyphenyl)-4-(N-methylindol-3-yl)thiophene, and di-tert-butylphenol-based compounds [e.g., DTPBHZ, DTPINH, DTPNHZ and DTPSAL]); and

analogs, derivatives and salts thereof.

Extracellular ATP stimulates and sensitizes endings of sensory nerves largely via binding to and activation of purinoceptors (including P2X3 and P2X2/3) on afferent nerve fibers innervating tissues (including the airways), resulting in intense sensations such as pain, itch, discomfort and urge (Weigand 2012). Therefore, antagonists of P2X purinergic receptors (e.g., P2X3 and P2X2/3 receptor antagonists) can be utilized to inhibit cough and urge to cough, including chronic cough (e.g., refractory chronic cough). Examples of antagonists of P2X purinergic receptors (e.g., P2X3 and P2X2/3 receptor antagonists) include without limitation pyridoxalphosphate-6-azophenyl-2′,4′-disulfonic acid (PPADS), suramin, AF-219, AF-130, BAY-1817080, substituted imidazopyridines of Formula (I) disclosed in US 2017/0326141 (e.g., BLU-5937), diaminopyrimidines disclosed in US 2016/0263112, and analogs, derivatives and salts thereof. In certain embodiments, the P2X antagonist is or includes AF-219, AF-130 or BAY-1817080.

Decongestants relieve nasal congestion by inducing, at low concentration, local vasoconstriction of the blood vessels in the nose, the throat and the paranasal sinuses, which reduces inflammation (swelling) and mucus formation in these areas. Non-limiting examples of decongestants include alpha (e.g., α₁ or/and α₂) adrenergic receptor agonists, such as ephedrine, levomethamphetamine, naphazoline, oxymetazoline, phenylephrine, phenylpropanolamine, propylhexedrine, pseudoephedrine, synephrine, tetryzoline (tetrahydrozoline), tramazoline, xylometazoline, and analogs, derivatives and salts thereof. In certain embodiments, the decongestant is or includes oxymetazoline or pseudoephedrine.

Beta (e.g., β₂) adrenoreceptor agonists can also be used as antitussives. β₂-adrenergic receptor agonists dilate the bronchi and bronchioles and inhibit histamine release from mast cells. Examples of short-acting β₂ agonists include without limitation bitolterol, fenoterol, isoprenaline (isoproterenol), levosalbutamol (levalbuterol), orciprenaline (metaproterenol), pirbuterol, procaterol, ritodrine, salbutamol (albuterol), terbutaline, and analogs, derivatives and salts thereof. Non-limiting examples of long-acting β₂ agonists include arformoterol, bambuterol, clenbuterol, formoterol, salmeterol, and analogs, derivatives and salts thereof. Examples of ultralong-acting β₂ agonists include without limitation carmoterol, indacaterol, milveterol, olodaterol, vilanterol, and analogs, derivatives and salts thereof. In certain embodiments, the β₂-agonist is or includes a short-acting β₂ agonist, such as procaterol, salbutamol or terbutaline. In further embodiments, the β₂-agonist is or includes a long-acting β₂ agonist, such as formoterol or salmeterol.

Acetylcholine can induce bronchoconstriction and cough (Ebihara 1995). Antagonists of muscarinic acetylcholine receptors (e.g., M1, M2, M3, M4 or/and M5) can dilate the bronchi and bronchioles and inhibit secretion by mucus glands regulated by the parasympathetic nervous system. Non-limiting examples of muscarinic antagonists include aclidinium (e.g., aclidinium bromide), atropine, benzatropine, biperiden, chlorpheniramine, cyclopentolate, darifenacin, dicyclomine, dimenhydrinate, diphenhydramine, doxepin, doxylamine, flavoxate, glycopyrrolate, hyoscyamine, ipratropium (e.g., ipratropium bromide), orphenadrine, oxitropium, oxybutynin, pentoxyverine (carbetapentane), pirenzepine, procyclidine, scopolamine (hyoscine), scopolamine butylbromide, scopolamine hydrobromide, solifenacin, tiotropium (e.g., tiotropium bromide), tolterodine, trihexyphenidyl, tropicamide, tricyclic antidepressants, and analogs, derivatives and salts thereof. In certain embodiments, the muscarinic antagonist is or includes atropine (an antagonist of M1, M2, M3, M4 and M5), ipratropium (a non-selective muscarinic antagonist) (e.g., ipratropium bromide), or tiotropium (a non-selective muscarinic antagonist) (e.g., tiotropium bromide).

Theobromine is another bronchodilator that can be used to treat bronchoconstriction, which causes coughing. Theobromine relaxes broncheal smooth muscle, and hence is useful for treating a bronchoconstrictive respiratory condition (e.g., asthma) and cough (e.g., chronic cough) associated therewith. Theobromine also exerts antitussive effect by suppressing vagal nerve activity. For instance, theobromine significantly increases the concentration of capsaicin required to induce cough.

Gastrin-releasing peptide (GRP) may mediate the involvement of C-fibers in the cough reflex (LaVinka 2013). GRP increases the pulmonary reflex response to capsaicin through activation of pulmonary C-fibers. In addition, the receptor for GRP, GRPR (aka bombesin receptor 2 [BBR2]), is present on airway (e.g., bronchopulmonary) epithelial cells, close to where C-fibers terminate. Therefore, antitussives may include inhibitors of gastrin-releasing peptide or the receptor therefor (GRPR or BBR2) or the production thereof, including but not limited to GRPR antagonists (e.g., RC-3095), and analogs, derivatives and salts thereof.

Examples of fever-reducing antipyretics include without limitation acetaminophen (paracetamol), metamizole, nabumetone, phenazone (antipyrine), NSAIDs (e.g., aspirin and related salicylates [e.g., choline salicylate, magnesium salicylate and sodium salicylate], ibuprofen, naproxen, ketoprofen and nimesulide), and analogs, derivatives and salts thereof. In certain embodiments, the antipyretic is or includes acetaminophen.

Examples of local anesthetics that decrease the sensitivity of stretch receptors (e.g., rapidly adapting stretch receptors) in the lower airways (including the lungs) and thereby reduce the urge to cough after taking a deep breath include without limitation benzonatate. Benzonatate can be used to reduce coughing associated with a respiratory condition, such as bronchitis, emphysema influenza or pneumonia. Other local anesthetics that can be used as antitussives, whether or not they have an effect on stretch receptors, include without limitation benzocaine and lidocaine.

Leukotriene receptor antagonists can be used to reduce airway inflammation that can lead to cough hypersensitivity, or to treat the underlying cough-associated medical conditions. Such conditions include respiratory conditions of a non-allergic or allergic character or marked by hypersensitivity, such as asthma and rhinitis. Leukotriene receptor antagonists include, but are not limited to, antagonists of cysteinyl leukotriene receptor 1 (cysLT1 or cysLTR1) (e.g., cinalukast, gemilukast [dual cysLT1/cysLT2 antagonist], iralukast, montelukast, pranlukast, tomelukast, verlukast, zafirlukast, CP-195494, CP-199330, ICI-198615 and MK-571) and cysLT2 antagonists (e.g., HAMI-3379), and analogs, derivatives and salts thereof. In certain embodiments, the leukotriene receptor antagonist is or includes montelukast or zafirlukast.

5-lipoxygenase (5-LOX) inhibitors are another class of leukotriene antagonists that inhibit the bronchoconstrictive, mucus-secreting and inflammatory effects of leukotrienes and hence can be used to reduce airway inflammation or to treat the underlying cough-associated respiratory condition such as asthma or rhinitis. 5-LOX inhibitors block the action of the arachidonate 5-LOX enzyme, which is responsible for the production of inflammatory leukotrienes. Examples of 5-LOX inhibitors include without limitation baicalein, caffeic acid, curcumin, hyperforin, meciofenamic acid, meclofenamate sodium, minocycline, zileuton, MK-886, and analogs, derivatives and salts thereof. In certain embodiments, the 5-LOX inhibitor is or includes zileuton.

Chronic cough is a common symptom of gastroesophageal reflux disease (GERD). Acidic stomach content is typically refluxed into the esophagus, although bland reflux can also provoke cough. A proton pump inhibitor can be used to decrease gastric acid production in GERD patients. Non-limiting examples of proton pump inhibitors include ilaprazole, lansoprazole, dexlansoprazole, omeprazole, esomeprazole, pantoprazole, rabeprazole, and analogs, derivatives and salts thereof. A prokinetic (gastroprokinetic or gastrokinetic) agent enhances gastrointestinal motility by increasing the frequency of contractions in the small intestine or making them stronger, without disrupting their rhythm. A prokinetic agent can be used to relieve gastrointestinal symptoms such as heart burn and to treat GERD. Non-limiting examples of prokinetic agents include benzamide, cinitapride, cisapride, domperidone, erythromycin, itopride, levosulpiride, metoclopramide, mitemcinal, mosapride, prucalopride, renzapride, tegaserod, and analogs, derivatives and salts thereof.

The underlying cause of neurogenic cough can be treated with, e.g., anticonvulsants or/and antidepressants. Anticonvulsants or/and antidepressants can also be used to treat chronic cough or cough hypersensitivity. In some embodiments, the anticonvulsants reduce neuronal excitability by, e.g., blocking a voltage-gated calcium or sodium channel, increasing the brain level of an inhibitory neurotransmitter (e.g., γ-aminobutyric acid [GABA]), activating a GABA receptor, or decreasing the brain level of an excitatory neurotransmitter (e.g., glutamate), or any combination thereof. Examples of anticonvulsants include without limitation carbamazepine, gabapentin, pregabalin, topiramate, valproic acid and salts thereof (e.g., sodium valproate), and analogs, derivatives and salts thereof. In certain embodiments, the anticonvulsant is or includes a GABA analog (e.g., gabapentin or pregabalin). Agonists of a GABA receptor (e.g., GABA-B) that do not have a significant anticonvulsant effect, such as baclofen, can also be used as antitussives. Spinal motor neurons are involved in cough generation (Bolser 2009). Therefore, compounds that suppress (e.g., expiratory) spinal motor activity, such as muscle relaxants (e.g., baclofen) and opioids (e.g., codeine), can inhibit cough. In certain embodiments, a neurokinin antagonist, such as an NK-1 antagonist (e.g., serlopitant, MK-0303 or MK-8478), is used in combination with a muscle relaxant (e.g., baclofen) or/and an opioid (e.g., codeine) to treat cough, including chronic cough. Examples of GABA-B agonists, regardless of whether or not they have a significant anticonvulsant effect, include without limitation GABA, γ-hydroxybutyrate (GHB), baclofen, phenibut, isovaline, 3-aminopropylphosphinic acid, 3-aminopropyl(methyl)phosphinic acid (SKF-97541), lesogaberan, CGP-44532, and analogs, derivatives and salts thereof. GABA-B agonists (e.g., lesogaberan) can also inhibit cough through peripheral action on bronchopulmonary vagal afferent nerves (Canning 2012).

Serotonin (5-hydroxytryptamine [5-HT]) and the serotonin precursor 5-hydroxytryptophan inhibit cough (Bolser 2009). Moreover, the serotonin precursor L-tryptophan can potentiate, and can prevent tolerance to, the antitussive effect of compounds, such as opioids (e.g., dihydrocodeine).

In some embodiments, antitussive antidepressants increase the level of serotonin in the brain, which can inhibit the central generating mechanism of the cough reflex (Stone 1993 and Kamei 1986). Non-limiting examples of antidepressants include tricyclic antidepressants (e.g., amitriptyline, amitriptylinoxide, amoxapine, dosulepin [dothiepin], doxepin, cidoxepin and melitracen), tetracyclic antidepressants (e.g., amoxapine, maprotiline, mazindol, mianserin, mirtazapine, esmirtazapine and setiptiline), selective serotonin reuptake inhibitors (SSRIs, e.g., citalopram, dapoxetine, escitalopram, fluoxetine, fluvoxamine, paroxetine, sertraline and S-41744), serotonin-norepinephrine reuptake inhibitors (SNRIs, e.g., bicifadine, doxepin, cidoxepin, duloxetine, milnacipran, levomilnacipran, sibutramine, venlafaxine, desvenlafaxine and SEP-227162), inhibitors of monoamine oxidases (MAOs) (e.g., selective MAO-A inhibitors [e.g., bifemelane, moclobemide, pirlindole {pirazidol} and toloxatone], selective MAO-B inhibitors [e.g., rasagiline and selegiline], and non-selective MAO-A/MAO-B inhibitors [e.g., hydracarbazine, isocarboxazid, nialamide, phenelzine and tranylcypromine]), and analogs, derivatives and salts thereof. In certain embodiments, the antidepressant is or includes an SSRI (e.g., fluvoxamine or paroxetine). In further embodiments, the antidepressant is or includes an SNRI (e.g., venlafaxine). In additional embodiments, the antidepressant is or includes a tricyclic antidepressant (e.g., amitriptyline, which also inhibits serotonin reuptake).

Activation of serotonin 5-HT_{1A} receptors in the brainstem can suppress cough, possibly via inhibition of current produced by activation of G protein-coupled inwardly-rectifying potassium channels (GIRKs) (Bolser 2009). Examples of 5-HT_{1A} agonists include without limitation alnespirone, befiradol, eptapirone, flibanserin, (±)-8-hydroxy-2-dipropylaminotetralin (8-OH-DPAT), lesopitron, osemozotan, repinotan, F-15599, LY-293284, MKC-242, U-92,016-A, and analogs, derivatives and salts thereof. In certain embodiments, the 5-HT_{1A} agonist is or includes 8-OH-DPAT.

The neurotrophins nerve growth factor (NGF) and brain-derived neurotrophic factor (BDNF) can induce transcriptional changes in sensory neurons that lead to their hyperinnervation (Keller 2017). Moreover, BDNF maintains an elevated level of neuronal excitation in part by suppressing inhibitory GABAergic signaling. NGF enhancement of cough in the CNS involves activation of tropomyosin kinase receptor A (TrkA), TRPV1 and NK-1 (El-Hashim 2013). Therefore, inhibition of the effects of NGF or BDNF can suppress the cough reflex. Examples of inhibitors of NGF or receptors therefor (e.g., TrkA and low-affinity NGF receptor [LNGFR]) or the production thereof include without limitation NGF inhibitors (e.g., fulranumab and tanezumab), TrkA antagonists (e.g., AG879, CT327 and K252a), and analogs, derivatives, fragments and salts thereof. Examples of inhibitors of BDNF or receptors thereof (e.g., TrkB and LNGFR) or the production thereof include without limitation TrkB antagonists (e.g., AZ623 and K252a), and analogs, derivatives and salts thereof.

Stimulation of the α7 nicotinic acetylcholine receptor (α7 nAChR) suppresses cough (Dicpinigaitis 2017). Therefore, α7 nAChR agonists can be used as antitussives to treat, e.g., acute and chronic cough. Examples of α7 nAChR agonists include without limitation nicotine, ATA-101 (TC-5619), PHA-543613, SEN-12333 (WAY-317538), azabicyclic aryl amides (e.g., PNU-282987 and compounds 1h, 1o, 2a, 9a and 18a disclosed in Walker [2006]), quinuclidine amides (e.g., those disclosed in Pin [2014]), and analogs, derivatives and salts thereof. In certain embodiments, the α7 nAChR agonist is or includes ATA-101 or PHA-543613.

The bulbs of Fritillaria plants (e.g., Bulbus Fritillariae Cirrhosae) contain alkaloids that can reduce cough frequency and increase the latent period of cough (Wang 2011). Examples of antitussive Fritillaria alkaloids include without limitation chuanbeinone, imperialine, verticine, verticinone, and analogs, derivatives and salts thereof.

The optional additional antitussive agent(s) can be administered to a subject suffering from cough or urge to cough, or a cough-associated condition, concurrently with (e.g., in the same composition as the neurokinin antagonist [e.g., an NK-1 antagonist, such as serlopitant, MK-0303 or MK-8478] or in separate compositions) or sequentially to (before or after) administration of the neurokinin antagonist. The neurokinin antagonist (e.g., an NK-1 antagonist, such as serlopitant, MK-0303 or MK-8478) and the optional additional antitussive agent(s) independently can be administered in any suitable mode, including without limitation orally, topically (e.g., dermally/epicutaneously, transdermally, mucosally, transmucosally, intranasally [e.g., by nasal spray or drop], pulmonarily [e.g., by oral or nasal inhalation], bucally, sublingually, rectally and vaginally), by injection or infusion (e.g., parenterally, including intramuscularly, subcutaneously, intradermally, intravascularly, intravenously, intra-arterially and intrathecally), and by implantation (e.g., subcutaneously and intramuscularly). In some embodiments, the neurokinin antagonist (e.g., an NK-1 antagonist, such as serlopitant, MK-0303 or MK-8478) or/and the optional additional antitussive agent(s) are administered by oral or nasal inhalation. In further embodiments, the neurokinin antagonist (e.g., an NK-1 antagonist, such as serlopitant, MK-0303 or MK-8478) or/and the optional additional antitussive agent(s) are administered intranasally (e.g., by nasal spray or drop). In further embodiments, the neurokinin antagonist (e.g., an NK-1 antagonist, such as serlopitant, MK-0303 or MK-8478) or/and the optional additional antitussive agent(s) are administered orally.

Examples of topical dosage forms include without limitation creams, ointments, gels, liniments, lotions, suppositories (e.g., rectal and vaginal suppositories), buccal and sublingual tablets and pills, sprays (e.g., dermal and nasal sprays), and drops (e.g., nose drops). Non-limiting examples of oral dosage forms include solid dosage forms (e.g., tablets, capsules and cachets) and liquid dosage forms (e.g., solutions or suspensions in an aqueous liquid or/and a non-aqueous liquid, and oil-in-water liquid emulsions or water-in-oil liquid emulsions). In a non-limiting example of a formulation for injection, the formulation is in the form of a solution and comprises an antitussive agent, a vehicle (e.g., a water-based vehicle or sterile water), a buffer, a reducing agent/antioxidant (e.g., sodium metabisulfite if epinephrine is used as a vasoconstrictor) and a preservative (e.g., methylparaben), and optionally a vasoconstrictor (e.g., epinephrine) to increase the duration of the pharmacological effect of the antitussive agent by constricting the blood vessels, thereby concentrating the antitussive agent for an extended duration and increasing the maximum dose of the antitussive agent. Other kinds of formulations for various modes of administration are described elsewhere herein.

The neurokinin antagonist (e.g., an NK-1 antagonist, such as serlopitant, MK-0303 or MK-8478) and the optional additional antitussive agent(s) independently can be administered in any suitable frequency, including without limitation daily (1, 2, 3, 4 or more times per day), every two or three days, twice weekly, once weekly, every two weeks, every three weeks, monthly, every two months or every three months, or in an irregular manner or on an as-needed basis. The dosing frequency can depend on, e.g., the mode of administration chosen. The length of treatment with the neurokinin antagonist (e.g., an NK-1 antagonist, such as serlopitant, MK-0303 or MK-8478) and the optional additional antitussive agent(s) can be determined by the treating physician and can independently be, e.g., at least about 1 week, 2 weeks, 3 weeks, 4 weeks (1 month), 6 weeks, 2 months, 3 months, 6 months, 1 year, 2 years, 3 years or longer.

### LITERATURE CITED

The following non-patent literature is cited herein:
Belvisi, M. and Hele, D., Handb. Exp. Pharmacol., 187:63-76 (2009);
Birrell, M. et al., Am. J. Respir. Crit. Care Med., 180:1042-1047 (2009);
Bolser, D., Handb. Exp. Pharmacol., 187:203-217 (2009);
Bonham, A. et al., J. Appl. Physiol., 81(4):1715-1722 (1996);
Canning, B. et al., Respir. Physiol. Neurobiol., 152:223-242 (2006);
Canning, B. et al., Am. J. Physiol. Regul. Integr. Comp. Physiol., 291(2):R454-R463 (2006);
Canning, B. et al., Cough, 8:7 (2012);
Chapman, R. et al., Drug News Perspect., 11(8):480-489 (1998);
Daoui, S. et al., Am. J. Respir. Crit. Care Med., 158(1):42-48 (1998);
Dicpinigaitis, P. et al., Eur. Respir. J., 50:OA4409 (2017);
Ebihara, T. et al., Am. J. Resp. Crit. Care Med., 151(3):815-821 (1995);
El-Hashim, A. et al., Pharmacol. Res., 74:68-77 (2013);
Jiang, J. et al., J. Med. Chem., 52(9):3039-3046 (2009);
Kamei, J. et al., Jpn J. Pharmacol., 42:531-538 (1986);
Kamei, J., Pul. Pharmacol., 9:349-356 (1996);
Keller, J. et al., Chest (Epub May 25, 2017);
LaVinka, P. et al., Cough, 9:8-20 (2013);
Mazzone, S. et al., J. Physiol., 569(Pt. 2):559-573 (2005);
Mazzone, S. et al., J. Neurosci., 29:13662-13671 (2009);
Mutoh, T. et al., Am. J. Physiol. Regul. Integr. Comp. Physiol., 279(4):R1215-R1223 (2000);
Park, H.-K. et al., Thorax, 61:1070-1075 (2006);
Pin, F. et al., Eur. J. Med. Chem., 82:214-224 (2014);
Stone, R. et al., J. Appl. Physiol., 74:396-401 (1993);
Walker, D. et al., Bioorg. Med. Chem., 14:8219-8248 (2006);
Wang, D. et al., Fitoterapia, 82:1290-1294 (2011); and
Weigand, L. et al., J. Physiol., 590(16):4109-4120 (2012).

### Representative Embodiments

The following embodiments of the disclosure are provided by way of illustration and example:

1. A method of treating cough or urge to cough, comprising administering to a subject in need of treatment a therapeutically effective amount of a neurokinin-1 (NK-1) antagonist, wherein the NK-1 antagonist is selected from serlopitant, MK-0303 (L-001182885), MK-8478 (L-001983867), NK-1 antagonists disclosed in U.S. Pat. No. 5,750,549, NK-1 antagonists disclosed in U.S. Pat. No. 8,124,633, and pharmaceutically acceptable salts, solvates, hydrates, clathrates, polymorphs, prodrugs, metabolites, stereoisomers and combinations thereof.
2. The method of embodiment 1, wherein the treating the cough or urge to cough comprises attenuating or suppressing the cough or urge to cough, or neuronal hypersensitivity underlying the cough or urge to cough.
3. The method of embodiment 1 or 2, wherein the cough is non-productive (dry) cough.
4. The method of any one of the preceding embodiments, wherein the NK-1 antagonist is or comprises serlopitant or a pharmaceutically acceptable salt, solvate, hydrate, clathrate, polymorph, prodrug, metabolite or stereoisomer thereof.
5. The method of any one of the preceding embodiments, wherein the NK-1 antagonist is or comprises MK-0303 (L-001182885) or a pharmaceutically acceptable salt, solvate, hydrate, clathrate, polymorph, prodrug, metabolite or stereoisomer thereof.
6. The method of any one of the preceding embodiments, wherein the NK-1 antagonist is or comprises MK-8478 (L-001983867) or a pharmaceutically acceptable salt, solvate, hydrate, clathrate, polymorph, prodrug, metabolite or stereoisomer thereof.
7. The method of any one of the preceding embodiments, wherein the NK-1 antagonist is or comprises the compound designated “Ex. #8” or the compound designated “Ex. #10” in U.S. Pat. No. 8,124,633, or a pharmaceutically acceptable salt, solvate, hydrate, clathrate, polymorph, prodrug, metabolite or stereoisomer thereof.
8. The method of any one of the preceding embodiments, wherein the therapeutically effective amount (e.g., per day or per dose) of the NK-1 antagonist (e.g., serlopitant, MK-0303 or MK-8478) is about 1-100 mg, 1-50 mg, 1-10 mg, 10-20 mg, 20-30 mg, 30-40 mg, 40-50 mg or 50-100 mg (e.g., about 1-10 mg).
9. The method of any one of the preceding embodiments, wherein the therapeutically effective amount of the NK-1 antagonist (e.g., serlopitant, MK-0303 or MK-8478) is administered one or more times a day, once every two days, once every three days, twice a week or once a week (e.g., once or twice daily).
10. The method of any one of the preceding embodiments, wherein the therapeutically effective amount of the NK-1 antagonist (e.g., serlopitant, MK-0303 or MK-8478) is about 1-5 mg (e.g., about 1 mg, 3 mg or 5 mg) once or twice daily, or about 5-10 mg (e.g., about 5 mg, 7.5 mg or 10 mg) once or twice daily.
11. The method of any one of the preceding embodiments, wherein the therapeutically effective amount of the NK-1 antagonist (e.g., serlopitant, MK-0303 or MK-8478) is administered over a period of at least about 2 weeks, 4 weeks (1 month), 6 weeks, 2 months, 3 months, 6 months, 1 year, 2 years, 3 years or longer (e.g., at least about 1, 2 or 3 months).
12. The method of any one of the preceding embodiments, wherein the NK-1 antagonist (e.g., serlopitant, MK-0303 or MK-8478) is administered orally (e.g., as a tablet or capsule).
13. The method of any one of embodiments 1 to 11, wherein the NK-1 antagonist (e.g., serlopitant, MK-0303 or MK-8478) is administered by oral or nasal inhalation (using, e.g., a metered-dose inhaler, a dry powder inhaler or a nebulizer).
14. The method of any one of embodiments 1 to 11, wherein the NK-1 antagonist (e.g., serlopitant, MK-0303 or MK-8478) is administered intranasally (e.g., by nasal spray, nose drop or pipette).
15. The method of any one of the preceding embodiments, wherein at least one loading dose of the NK-1 antagonist (e.g., serlopitant, MK-0303 or MK-8478) is first administered, and at least one therapeutically effective maintenance dose of the NK-1 antagonist is subsequently administered.
16. The method of embodiment 15, wherein the at least one therapeutically effective maintenance dose (e.g., per day or per dose) of the NK-1 antagonist (e.g., serlopitant, MK-0303 or MK-8478) is about 1-100 mg, 1-50 mg, 1-10 mg, 10-20 mg, 20-30 mg, 30-40 mg, 40-50 mg or 50-100 mg (e.g., about 1-10 mg).
17. The method of embodiment 15 or 16, wherein the at least one loading dose of the NK-1 antagonist (e.g., serlopitant, MK-0303 or MK-8478) is about 1.5, 2, 3, 4 or 5 times (e.g., about 3 times) greater than the at least one therapeutically effective maintenance dose of the NK-1 antagonist.
18. The method of any one of embodiments 15 to 17, wherein the at least one therapeutically effective maintenance dose of the NK-1 antagonist (e.g., serlopitant, MK-0303 or MK-8478) is administered one or more times a day, once every two days, once every three days, twice a week or once a week (e.g., once or twice daily).
19. The method of any one of embodiments 15 to 18, wherein the at least one therapeutically effective maintenance dose of the NK-1 antagonist (e.g., serlopitant, MK-0303 or MK-8478) is administered over a period of at least about 2 weeks, 4 weeks (1 month), 6 weeks, 2 months, 3 months, 6 months, 1 year, 2 years, 3 years or longer (e.g., at least about 1, 2 or 3 months).
20. The method of any one of embodiments 15 to 19, wherein the NK-1 antagonist (e.g., serlopitant, MK-0303 or MK-8478) is administered in a loading dose of about 3-15 mg or 15-30 mg once or twice on day 1, followed by a maintenance dose of about 1-5 mg or 5-10 mg once or twice daily for at least about 2 weeks, 1 month, 2 months, 3 months, 6 months, 1 year, 2 years, 3 years or longer (e.g., at least about 1, 2 or 3 months), where the loading dose is three times larger than the maintenance dose and the NK-1 antagonist is administered orally (e.g., as a tablet or capsule), pulmonarily (e.g., by oral or nasal inhalation) or intranasally (e.g., by nasal spray or drop).
21. The method of any one of the preceding embodiments, wherein the NK-1 antagonist (e.g., serlopitant, MK-0303 or MK-8478) is administered at bedtime or in the morning.
22. The method of any one of the preceding embodiments, wherein the NK-1 antagonist (e.g., serlopitant, MK-0303 or MK-8478) is administered without food (e.g., at least about 1 or 2 hours before or after a meal, such as at least about 2 hours after an evening meal or at least about 2 hours before or after a meal in the morning).
23. The method of any one of the preceding embodiments, wherein treatment with the NK-1 antagonist (e.g., serlopitant, MK-0303 or MK-8478) reduces the frequency (e.g., the number of coughs per hour during daytime, awake hours, sleep or the whole day), the severity (e.g., visual analog scale [VAS] or cough severity diary [CSD]) or the impact (e.g., Leicester cough questionnaire [LCQ] or cough-specific quality of life questionnaire [CQLQ]), or any combination or all thereof, of the cough or urge to cough, by at least about 20%, 30%, 40%, 50%, 60%, 70%, 80%, 90% or 95% (e.g., by at least about 30% or 50%).
24. The method of any one of the preceding embodiments, wherein the cough or urge to cough is chronic cough (e.g., idiopathic chronic cough or refractory/treatment-resistant chronic cough).
25. The method of any one of the preceding embodiments, wherein the cough or urge to cough is associated with a respiratory condition, a lung tissue disorder, gastroesophageal reflux disease (GERD), or post-nasal drip.
26. The method of any one of the preceding embodiments, further comprising administering one or more additional antitussive agents.
27. The method of embodiment 26, wherein the one or more additional antitussive agents are selected from NK-2 antagonists, NK-3 antagonists, mixed NK antagonists, opioids (e.g., mu-opioid receptor agonists and kappa-opioid receptor agonists), NOP/ORL-1 receptor agonists, agonists of sigma (e.g., σ₁ or/and σ₂) receptors, NMDAR antagonists, cannabinoid receptor type 2 (CB₂) agonists, butamirate class of antitussives, TRPV1 antagonists, TRPV4 antagonists, TRPA1 antagonists, inhibitors of bradykinin or receptors therefor (e.g., B1 and B2), inhibitors of inflammatory prostaglandins (e.g., PGE2) or receptors therefor (e.g., EP3), inhibitors of calcitonin gene-related peptide (CGRP) or receptor therefor, antagonists of protease-activated receptors (PARs) and inhibitors of activating proteases, anti-inflammatory agents (e.g., antihistamines, mast cell stabilizers, corticosteroids, immunomodulators, non-steroidal anti-inflammatory drugs, leukotriene receptor antagonists and 5-lipoxygenase inhibitors), antagonists of P2X purinergic receptors (e.g., P2X3 and P2X2/3 receptor antagonists), decongestants, beta (e.g., β₂) adrenergic receptor agonists, antagonists of muscarinic acetylcholine receptors (e.g., M1, M2, M3, M4 or/and M5), inhibitors of gastrin releasing peptide (GRP) or the receptor therefor (GRPR or BBR2), antipyretics, anticonvulsants, GABA-B receptor agonists, antidepressants, 5-HT_{1A} agonists, inhibitors of nerve growth factor (NGF) or receptors therefor (e.g., TrkA and LNGFR), inhibitors of brain-derived neurotrophic factor (BDNF) or receptors thereof (e.g., TrkB and LNGFR), α7 nicotinic acetylcholine receptor agonists, Fritillaria alkaloids, peripheral antitussives (e.g., dropropizine [dipropizine], levodropropizine, moguisteine [inhibitor of rapidly adapting receptors], and naringin [inhibitor of substance P content and NK-1 expression]), local anesthetics, vitamins (e.g., vitamin C), minerals (e.g., zinc), sweet substances (e.g., honey and sugar syrup), and therapeutic agents that treat the underlying cause of the cough or urge to cough, including but not limited to antihistamines for putative post-nasal drips; corticosteroids (e.g., prednisone) or/and bronchodilators (e.g., β₇-adrenoreceptor agonists) for putative asthma; leukotriene receptor antagonists or/and mast cell stabilizers (e.g., cromoglicic acid and nedocromil) for putative asthma; corticosteroids for putative NAEB; corticosteroids or/and bronchodilators (e.g., β₂-adrenoreceptor agonists) for putative COPD; first-generation antihistamines with anticholinergic activity, bronchodilators (e.g., ipratropium bromide) or/and decongestants (e.g., oxymetazoline hydrochloride) for putative UACS; decongestants (e.g., pseudoephedrine) or/and antibiotics for putative bacterial sinusitis; antibiotics for putative bacterial bronchitis, pertussis or tuberculosis; proton-pump inhibitors or/and prokinetic agents for putative GERD; and anticonvulsants or/and tricyclic antidepressants for neurogenic cough.
28. The method of embodiment 26 or 27, wherein the one or more additional antitussive agents are administered pulmonarily (e.g., by oral or nasal inhalation) or intranasally (e.g., by nasal spray or drop).
29. The method of any one of embodiments 26 to 28, wherein the one or more additional antitussive agents are administered orally.
30. An NK-1 antagonist selected from serlopitant, MK-0303, MK-8478, NK-1 antagonists disclosed in U.S. Pat. No. 5,750,549, NK-1 antagonists disclosed in U.S. Pat. No. 8,124,633, and pharmaceutically acceptable salts, solvates, hydrates, clathrates, polymorphs, prodrugs, metabolites and stereoisomers thereof for use in the treatment of cough or urge to cough, optionally in combination with an additional antitussive agent.
31. A composition comprising an NK-1 antagonist selected from serlopitant, MK-0303, MK-8478, NK-1 antagonists disclosed in U.S. Pat. No. 5,750,549, NK-1 antagonists disclosed in U.S. Pat. No. 8,124,633, and pharmaceutically acceptable salts, solvates, hydrates, clathrates, polymorphs, prodrugs, metabolites and stereoisomers thereof for use in the treatment of cough or urge to cough, optionally in combination with an additional antitussive agent.
32. Use of an NK-1 antagonist selected from serlopitant, MK-0303, MK-8478, NK-1 antagonists disclosed in U.S. Pat. No. 5,750,549, NK-1 antagonists disclosed in U.S. Pat. No. 8,124,633, and pharmaceutically acceptable salts, solvates, hydrates, clathrates, polymorphs, prodrugs, metabolites and stereoisomers thereof in the preparation of a medicament for the treatment of cough or urge to cough, optionally in combination with an additional antitussive agent.
33. A kit comprising:

an NK-1 antagonist selected from serlopitant, MK-0303, MK-8478, NK-1 antagonists disclosed in U.S. Pat. No. 5,750,549, NK-1 antagonists disclosed in U.S. Pat. No. 8,124,633, and pharmaceutically acceptable salts, solvates, hydrates, clathrates, polymorphs, prodrugs, metabolites and stereoisomers thereof; and

instructions for using the NK-1 antagonist to treat cough or urge to cough.

34. The kit of embodiment 33, further comprising an inhaler.
35. A method of treating cough or urge to cough, comprising administering to a subject in need of treatment a therapeutically effective amount of a neurokinin-1 (NK-1) antagonist and a therapeutically effective amount of an N-methyl-D-aspartate receptor (NMDAR) antagonist.
36. The method of embodiment 35, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, SCH-720881, serlopitant, tradipitant, vestipitant, vofopitant, hydroxyphenyl propamidobenzoic acid, maltooligosaccharides (e.g., maltotetraose and maltopentaose), spantides (e.g., spantide I and II), AV-608, AV-818, AZD-2624, BIIF 1149 CL, CGP-49823, CJ-17493, CP-96345, CP-99994, CP-122721, DNK-333, FK-224, FK-888, GR-82334, GR-205171, GSK-424887, HSP-117, KRP-103, L-703606, L-733060, L-736281, L-759274, L-760735, LY-686017, M516102, MDL-105212, MK-0303, MK-8478, NKP-608, R-116031, R-116301, RP-67580, S-41744, SCH-206272, SCH-388714, SCH-900978, SLV-317, SSR-240600, T-2328, TA-5538, TAK-637, TKA-731, WIN-51708, ZD-4974, ZD-6021, cycloalkyl (including cyclopentyl, cyclohexyl and cycloheptyl) tachykinin receptor antagonists disclosed in U.S. Pat. No. 5,750,549, hydroxymethyl ether hydroisoindoline tachykinin receptor antagonists disclosed in U.S. Pat. No. 8,124,633, and pharmaceutically acceptable salts, solvates, hydrates, clathrates, polymorphs, prodrugs, metabolites, stereoisomers and combinations thereof.
37. The method of embodiment 35 or 36, wherein the NK-1 antagonist is or comprises serlopitant, MK-0303 or MK-8478, or a pharmaceutically acceptable salt, solvate, hydrate, clathrate, polymorph, prodrug, metabolite or stereoisomer thereof.
38. The method of embodiment 37, wherein the NK-1 antagonist is or comprises serlopitant or a pharmaceutically acceptable salt, solvate, hydrate, clathrate, polymorph, prodrug, metabolite or stereoisomer thereof.
39. The method of any one of embodiments 35 to 38, wherein the NMDAR antagonist is an uncompetitive antagonist (or channel blocker) that has a moderate affinity for the dizocilpine/phencyclidine-binding site in the NMDAR channel.
40. The method of any one of embodiments 35 to 39, wherein the NMDAR antagonist is selected from alaproclate, amantadine, atomoxetine, budipine, delucemine, dextrallorphan, dextromethorphan, dextrorphan, dexanabinol, eliprodil, ketamine, lanicemine, minocycline, memantine, nitromemantine, NEFA (a tricyclic small molecule), neramexane, orphenadrine, procyclidine, ARL/FPL U495/12495AA (desglycine metabolite of remacemide), and pharmaceutically acceptable salts, solvates, hydrates, clathrates, polymorphs, prodrugs, metabolites, stereoisomers and combinations thereof.
41. The method of embodiment 40, wherein the NMDAR antagonist is or comprises memantine, nitromemantine, amantadine, lanicemine, neramexane, dextrallorphan, dextromethorphan or procyclidine, or a pharmaceutically acceptable salt, solvate, hydrate, clathrate, polymorph, prodrug, metabolite or stereoisomer thereof.
42. The method of embodiment 41, wherein the NMDAR antagonist is or comprises memantine, nitromemantine, dextrallorphan or dextromethorphan, or a pharmaceutically acceptable salt, solvate, hydrate, clathrate, polymorph, prodrug, metabolite or stereoisomer thereof.
43. The method of any one of embodiments 35 to 42, wherein the treating the cough or urge to cough comprises attenuating or suppressing the cough or urge to cough, or neuronal hypersensitivity underlying the cough or urge to cough.
44. The method of any one of embodiments 35 to 43, wherein the cough is non-productive (dry) cough.
45. The method of any one of embodiments 35 to 44, wherein the cough or urge to cough is chronic cough (e.g., idiopathic chronic cough or refractory/treatment-resistant chronic cough).
46. The method of any one of embodiments 35 to 45, wherein the cough or urge to cough is associated with a respiratory condition, a lung tissue disorder, gastroesophageal reflux disease (GERD), or post-nasal drip.
47. The method of any one of embodiments 35 to 46, further comprising administering one or more additional antitussive agents.
48. An NK-1 antagonist for use in the treatment of cough or urge to cough, in combination with an NMDAR antagonist for use in the treatment of cough or urge to cough.
49. A composition comprising an NK-1 antagonist for use in the treatment of cough or urge to cough, in combination with a composition comprising an NMDAR antagonist for use in the treatment of cough or urge to cough.
50. Use of an NK-1 antagonist in the preparation of a medicament for the treatment of cough or urge to cough, in combination with use of an NMDAR antagonist in the preparation of a medicament for the treatment of cough or urge to cough.

### EXAMPLES

The following examples are intended only to illustrate the disclosure. Other assays, studies, protocols, procedures, methodologies, materials, substances, reagents and conditions may alternatively be performed or used as appropriate. All of the inactive pharmaceutical ingredients in the examples below comply with United States Pharmacopeia and The National Formulary requirements and are tested and released according to the monograph for each ingredient specified in the USP/NF compendium.

### Example 1. Preparation of Serlopitant Tablets

The NK-1 antagonist serlopitant can be formulated as a tablet for oral use. Table 1 shows qualitative/quantitative composition of exemplary dosages. Minor variations in the excipient quantities (+/−10%) may occur during the drug development process.

TABLE 1

| Components | Function | % of composition |
|---|---|---|
| Serlopitant | Active agent | 1-6% |
| Microcrystalline cellulose | Diluent | 50-60% |
| Mannitol | Diluent | 20-30% |
| Croscarmellose Sodium | Disintegrant | 1-3% |
| Colloidal silica | Disintegrant | 0.25-0.5% |
| Sodium Lauryl Sulfate | Surfactant | 5-6% |
| Magnesium Stearate | Lubricant | 0.25-2% |

| | |
|---|---|
| Total Tablet Composition | 100% |

Tablet potencies of 0.25 mg, 1 mg and 5 mg are prepared as a compressed tablet formulation. The tablet manufacturing process is the same for all potencies. The process comprises the following steps: 1) serlopitant, mannitol and sodium lauryl sulfate are blended; 2) the remaining mannitol is added to the blender and mixed; 3) microcrystalline cellulose, croscarmellose sodium and colloidal silica are added to the blender containing the mixture above to complete the mixing, and the blend is de-agglomerated if necessary; 4) the blend is lubricated with magnesium stearate that has been previously screened, if necessary; 5) the lubricated blend is roller-compacted and milled, and then lubricated with magnesium stearate that has been previously screened, if necessary; and 6) the mixture is compressed into tablets of the appropriate weight.

Similar tablets can be prepared for other NK-1 antagonists (e.g., MK-0303 and MK-8478).

### Example 2. Preparation of Serlopitant Capsules

Serlopitant can also be formulated as liquid-filled capsules. Table 2 shows qualitative/quantitative composition of exemplary dosages. Minor variations in the excipient quantities (+/−10%) may occur during the drug development process.

TABLE 2

| | | Unit Strength | | | | | |
|---|---|---|---|---|---|---|---|
| Components | Function | 0.25 mg | | 1 mg | | 4 mg | |
| Capsule Fill | | | | | | | |
| Serlopitant | Active agent | 0.25 | mg | 1 | mg | 4 | mg |
| Mono- & | Solubilizer | 399 | mg | 398.6 | mg | 395.6 | mg |
| Di-glycerides | | | | | | | |
| Butylated | Antioxidant | 0.40 | mg | 0.40 | mg | 0.40 | mg |
| Hydroxyanisole | | | | | | | |
| Capsule Shell | | | | | | | |
| #0 White Opaque | Capsule shell | 96 | mg** | 96 | mg** | 96 | mg** |
| Hard Gelatin Capsule* | | | | | | | |

| | | | | |
|---|---|---|---|---|
| Gelatin*** | Banding component | — | — | — |
| Polysorbate 80*** | Banding component | — | — | — |

*Capsules are provided by Capsugel (Morristown, NJ) and contain gelatin and titanium dioxide
**Approximate weight of empty capsule shell
***As needed to seal the capsule shells

The formulation is prepared by dissolving the drug substance in mono- and di-glycerides. Furthermore, 0.1 w t butylated hydroxyanisole is added as an antioxidant. Initial capsule strengths are dispensed into hard gelatin capsules and sealed by spraying with a 1:1 (wt/wt) water:ethanol solution. Subsequent potencies, including 0.25 mg, 1 mg and 4 mg, are dispensed into hard gelatin capsules and sealed with a band of gelatin/polysorbate 80. Corresponding placebo formulations are prepared in a similar manner, but without the addition of the drug substance and the antioxidant.

The capsule manufacturing process is the same for all potencies. The process comprises the following steps: 1) the mono- and di-glycerides are melted at 40° C., if necessary; 2) the mono- and di-glycerides are added to an appropriately sized, jacketed vessel and mixing is initiated; 3) the butylated hydroxyanisole is added to the mono- and di-glycerides and mixed until dissolved (minimum of 10 min); 4) serlopitant is slowly added to the mixture and mixed until dissolved (visual confirmation); 5) the solution is filled into hard gelatin capsules; 6) the filled capsules are sealed with a mixture of gelatin and polysorbate 80; 7) the sealed capsules are allowed to dry overnight and then the capsules are visually inspected for leaking; 8) the acceptable capsules may be weight-sorted, if necessary; and 9) the finished product is packaged in appropriate containers.

Similar capsules can be prepared for other NK-1 antagonists (e.g., MK-0303 and MK-8478).

### Example 3. Topical Formulations Containing Serlopitant

Table 3 shows various topical formulations containing serlopitant. The formulations contain Vanicream™ Moisturizing Skin Cream (“VM”), Vanicream™ Lite Lotion (“VLL”) or Aquaphor® Healing Ointment (“AP”, from Eucerin) as the base or carrier. VM and VLL are oil-in-water emulsion and AP has an oil base. A stock solution of free base serlopitant (Compound 1, or “Cpd 1”, in Tables 3 and 4) in ethanol (EtOH) was prepared by dissolving free base serlopitant in ethanol to the maximum extent and then filtering the resulting solution through an Anotop® 25 inorganic filter having a 0.02 micron pore size. Free base serlopitant has a maximum solubility in ethanol of 64.5 mg/g EtOH, or 6.45% w/w. To prepare a topical formulation, the stock solution of serlopitant/ethanol was added to a tared tube containing a particular amount of the base until the resulting mixture weighed 25.0 g. The mixture was mixed vigorously for 2 minutes using a vibration stand and then was rotated slowly for 4 days. For the “C” formulations, ethanol containing no serlopitant was added so that the “B” and “C” formulations would contain the same amount of base and ethanol.

TABLE 3

| | | | Cpd 1/EtOH | Blank | | |
|---|---|---|---|---|---|---|
| | Lot Size | Base | Stock Soln | EtOH | % Cpd 1 | % EtOH |
| Mixture | (g) | (g) | (g) | (g) | (w/w) | (w/w) |
| VM-A | 25.0 | 23.06 | 1.94 | 0.0 | 0.5 | 7.8 |
| VM-B | 25.0 | 21.12 | 3.88 | 0.0 | 1.0 | 15.5 |
| VM-C | 25.0 | 21.12 | 1.94 | 1.94 | 0.5 | 15.5 |
| VLL-A | 25.0 | 23.06 | 1.94 | 0.0 | 0.5 | 7.8 |
| VLL-B | 25.0 | 21.12 | 3.88 | 0.0 | 1.0 | 15.5 |
| VLL-C | 25.0 | 21.12 | 1.94 | 1.94 | 0.5 | 15.5 |
| AP-A | 25.0 | 23.06 | 1.94 | 0.0 | 0.5 | 7.8 |
| AP-B | 25.0 | 21.12 | 3.88 | 0.0 | 1.0 | 15.5 |
| AP-C | 25.0 | 21.12 | 1.94 | 1.94 | 0.5 | 15.5 |

AP was determined to be an unsuitable base for an ethanol solution containing serlopitant because of ethanol insolubility in that base. The VM base appeared stable/unchanged under 15× microscopic magnification after 4 days of mixing with 15.5% ethanol. The VLL base showed some aggregation of lamellar structures under 15× microscopic magnification after 4 days of mixing with 15.5% ethanol, but the overall change to the base appeared minor. The VM and VLL formulations can be tested, e.g., for the skin permeation of serlopitant.

Similar topical formulations can be prepared for other NK-1 antagonists (e.g., MK-0303 and MK-8478).

### Example 4. In Vitro Skin Permeation of Serlopitant in Topical Formulations

Topical formulations A-D used in the in vitro skin permeation studies are shown in Table 4. The bases “VM” and “VLL” of formulations A-D are described in Example 3. Formulations A-D were prepared according to the procedures described in Example 3.

TABLE 4

| | Final | | Cpd 1/EtOH | Blank | | |
|---|---|---|---|---|---|---|
| Formul'n | Mass | Base | Stock Soln | EtOH | % Cpd 1 | % EtOH |
| (Base) | (g) | (g) | (g) | (g) | (w/w) | (w/w) |
| A (VM) | 25.28 | 21.27 | 0.0 | 4.01 | 0.0 | 15.9 |
| B (VLL) | 25.12 | 21.19 | 3.93 | 0.0 | 1.0 | 15.6 |
| C (VM) | 13.80 | 11.63 | 2.17 | 0.0 | 1.0 | 15.7 |
| D (VLL) | 25.02 | 21.15 | 0.0 | 3.87 | 0.0 | 15.5 |

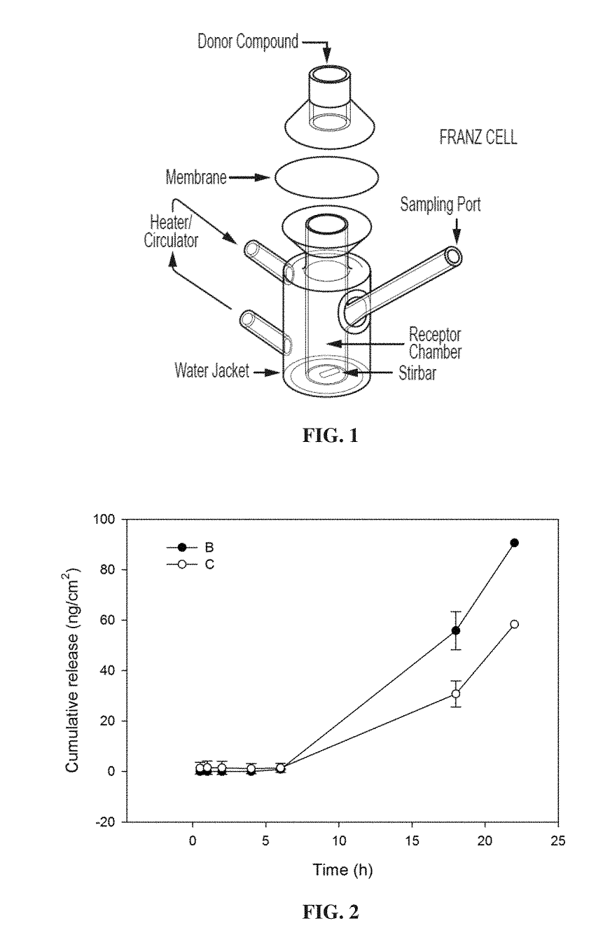

In vitro skin permeation of serlopitant in topical formulations A-D was evaluated using a Franz diffusion cell. FIG. 1 illustrates a Franz diffusion cell. A Franz diffusion cell having a circular permeation area of 4.15 cm² and a receptor chamber volume of 19 mL was set up with a thermo-regulated outer water jacket to maintain the temperature at 37° C. The receptor chamber was filled with 19 mL 1×PBS (pH 7.5) containing 10% ethanol and 1% Tween® 80. Solubility test indicated that serlopitant remained soluble at concentrations of 0.5, 5 and 50 ug/mL in this solution after 1 hour of incubation at 37° C. The solubility of serlopitant decreased significantly if Tween® 80 was not used and decreased slightly if ethanol was not used.

Human skin was pre-treated to remove all subcutaneous fat and was cleaned with 70% ethanol before use. The skin was visually inspected to ensure that it was free of any surface irregularity or small holes and was equally divided into four pieces. The skin was then mounted onto the receptor chamber with the stratum corneum side facing up. About 100 mg of topical formulation A, B, C or D was applied to the skin (actual weight: A, 103.8 mg; B, 101.3 mg; C, 103.2 mg; and D, 103.8 mg), which was then covered with parafilm to avoid evaporation.

About 0.5 mL of solution was withdrawn through the sampling port of the Franz diffusion cell at 0.5, 1, 2, 4, 6, 18 and 22 hours. The receptor chamber was replenished with equal volume of fresh diffusion buffer after each sampling. At the end of the experiment (after 22 hours of incubation), the skin was wiped clean with methanol, and the formulation-treated area was weighed and frozen for cryosectioning.

All samples were processed by solid-phase extraction (SPE) before LC-MS/MS analysis. Briefly, a Strata-X 33 um Polymeric Reverse-Phase column with 30 mg sorbent mass/1 mL volume (Phenomenex) was conditioned with 1 mL of methanol and equilibrated with 1 mL of water. 300 uL of sample was loaded to the column followed by a wash with 1 mL of 30% methanol. Serlopitant was eluted with 2% formic acid in acetonitrile. The sample then was concentrated by blow drying with nitrogen and re-suspended in 50 uL of 50% methanol. A working standard was first generated by spiking the diffusion buffer with known concentrations of serlopitant, which was then processed using the same SPE method. A sensitivity of 0.1 ng/mL was achieved. Serlopitant concentrations in samples resulting from formulations A-D were determined by comparison to the standard. Serlopitant was not detected in samples resulting from topical formulations A and D, as expected. FIG. 2 shows the cumulative release of serlopitant from topical formulations B and C into the receptor chamber at 0.5, 1, 2, 4, 6, 18 and 22 hours. After an initial lag, serlopitant was detected by LC-MS/MS in the receptor chamber at 6 hours. FIG. 2 indicates that topical formulation B resulted in greater penetration of serlopitant through the skin than topical formulation C in this in vitro study.

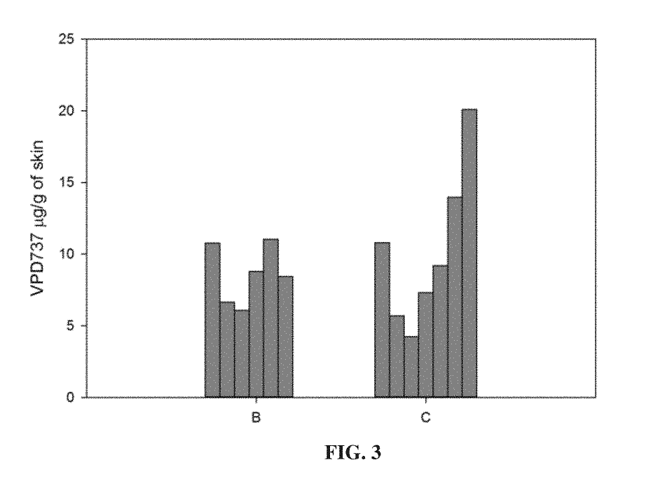

The amount of serlopitant retained in the skin was determined at the end of the experiment. The skin was wiped and washed with methanol. The formulation-treated area was cut into horizontal sections of 25 um using a cryostat. Every 10 sections were pooled, placed in Eppendorf tubes, weighed and digested with twice the volume of 1 mg/mL liberase at 37° C. for 1 hour. Digested skin sections were further homogenized with a probe sonicator. To 25 uL of the skin homogenate were added 25 uL of 50% methanol and 100 uL of acetonitrile/methanol to extract serlopitant. For spiked standards, 25 uL of a solution of serlopitant in 50% methanol (from 5 ng/mL to 5000 ng/mL) was added to 25 uL of blank skin homogenate followed by 100 uL of acetonitrile/methanol. Extracted serlopitant was quantified by LC-MS/MS. FIG. 3 shows the amount of serlopitant (called “VPD737” in FIG. 3) retained in the skin at the end of the experiment. Each bar represents ug of serlopitant/g of skin in 250 um skin layers. For each of topical formulations B and C, the bars from left to right represent the amount of serlopitant retained in skin layers from the stratum corneum to the dermis.

Similar in vitro skin permeation studies can be conducted for other NK-1 antagonists (e.g., MK-0303 and MK-8478).

### Example 5. Representative Topical Formulations Containing an NK-1 Antagonist

Table 5 provides non-limiting examples of topical formulations that can be prepared with an NK-1 antagonist (e.g., serlopitant, MK-0303 or MK-8478) or a salt, solvate, hydrate, clathrate, polymorph, prodrug or metabolite thereof, and optionally an additional therapeutic (e.g., antitussive) agent.

TABLE 5

| Dosage | Ingredients in Addition to NK-1 Antagonist |
|---|---|
| Form | (e.g., Serlopitant, MK-0303 or MK-8478) |
| inhalation | lactose, starch, a starch derivative |
| (DPI) | (e.g., hydroxypropylmethyl cellulose) or |
| | polyvinylpyrrolidine, and optionally |
| | magnesium stearate or/and leucine |
| inhalation | a propellant (e.g., 1,1,1,2-tetrafluoroethane), |
| (MDI) | a surfactant (e.g., lecithin or oleic |
| | acid), and a co-solvent (e.g., ethanol) |
| inhalation | polysorbate 80, edetate disodium, sodium |
| (nebulizer) | chloride, pH buffering agents (e.g., citric |
| | acid/sodium citrate), and water |
| spray | microcrystalline cellulose, sodium |
| (nasal) | carboxymethylcellulose, dextrose, water, and |
| | optionally a pH adjuster (e.g., HCl) |
| spray | microcrystalline cellulose, carboxymethyl |
| (nasal) | cellulose sodium, dextrose, polysorbate |
| | 80, disodium edetate, potassium sorbate, |
| | a pH adjuster (e.g., HCl), water, and |
| | optionally an alcohol (e.g., ethanol) |
| spray | microcrystalline cellulose, carboxymethyl |
| (nasal) | cellulose sodium, dextrose, polysorbate |
| | 80, benzalkonium chloride, phenylethyl alcohol, |
| | water, and optionally an alcohol |
| | (e.g., ethanol) |
| spray | hypromellose, benzalkonium chloride, NaCl, |
| (nasal) | EDTA, citric acid, sodium phosphate |
| | dibasic, water, and optionally an alcohol |
| | (e.g., ethanol) |
| cream | sorbitol, cetyl alcohol, isopropyl myristate, |
| | glyceryl stearate, PEG-100 stearate, |
| | petrolatum, benzyl alcohol, titanium |
| | dioxide and water |
| cream | propylene glycol, cetostearyl alcohol, |
| | Cremophor ® A6, Cremophor ® A25, liquid |
| | paraffin, parabens and water |
| cream | glycerol, sorbitol, isopropyl palmitate, |
| | emulsifying wax, benzyl alcohol, a pH |
| | adjuster (e.g., NaOH or lactic acid), and water |
| cream | glycerol, stearic acid, glyceryl monostearate, |
| | triethanolamine, parabens and water |
| cream | propylene glycol, cetostearyl alcohol, mineral |
| | oil, white petrolatum, ceteareth-30, |
| | chlorocresol, sodium phosphate monobasic, |
| | phosphoric acid, water, and optionally |
| | NaOH |
| cream | glycerol, cetostearyl alcohol, mineral oil, |
| | petrolatum, ceteth-20, diazolidinyl urea, |
| | dichlorobenzyl alcohol, edetic acid (EDTA) |
| | or disodium edetate, dibasic sodium |
| | phosphate and water |
| cream | propylene glycol, stearyl alcohol, white |
| | petrolatum, polysorbate 60, parabens, and |
| | optionally water |
| cream | propylene glycol, stearyl alcohol, cetyl |
| | alcohol, oleyl alcohol, mono-, di- or/and |
| | tri-glycerides, sodium cetostearyl sulphate, |
| | benzyl alcohol, citric acid, a pH adjuster |
| | (e.g., NaOH or lactic acid), and water |
| cream | hexylene glycol, stearyl alcohol, propylene |
| | glycol stearate, white wax, white |
| | petrolatum, aluminum starch octenylsuccinate, |
| | ceteareth-20, titanium dioxide, |
| | phosphoric acid and water |
| cream | propylene glycol, sorbitol, glyceryl |
| | monoisostearate, polyglyceryl-3 oleate, |
| | mineral oil, microcrystalline wax, |
| | colloidal silicon dioxide, parabens, EDTA or |
| | disodium edetate, and water |
| cream | propylene glycol, stearic acid, isopropyl |
| | palmitate, emulsifying wax, beeswax, |
| | polysorbate 60, an antioxidant (e.g., |
| | propyl gallate), a preservative (e.g., sorbic |
| | acid or/and potassium sorbate), a pH adjuster |
| | (e.g., NaOH or/and citric acid), and water |
| cream | cetostearyl alcohol, lanolin alcohols, |
| | isopropyl myristate, aluminum stearate, |
| | magnesium stearate, mineral oil, white |
| | petrolatum, water, and optionally disodium |
| | edetate or/and lactic acid |
| cream | propylene glycol, cetostearyl alcohol, |
| | white soft paraffin, liquid paraffin, lanolin, |
| | simethicone M30, Tween ® 60, |
| | parabens and water |
| cream | cetostearyl alcohol, mineral oil, white |
| | petrolatum, ceteth-20, parabens, citric |
| | acid, sodium citrate, and water |
| cream | propylene glycol, cetostearyl alcohol, |
| | polyoxyl 20 cetostearyl ether, mineral oil |
| | (liquid paraffin), petrolatum (white soft |
| | paraffin), chlorocresol, parabens, sodium |
| | phosphate monobasic, and water |
| cream | propylene glycol, cetostearyl alcohol, |
| | stearic acid, cetyl palmitate, sorbitan |
| | monostearate, mineral oil, polysorbate |
| | 60, benzyl alcohol and water |
| ointment | hexylene glycol, propylene glycol |
| | stearate, white wax, white petrolatum, |
| | phosphoric acid and water |
| ointment | propylene glycol, mineral oil, petrolatum, |
| | steareth-2, tocopherol, EDTA or |
| | disodium edetate, dibasic sodium |
| | phosphate and water |
| ointment | propylene glycol, fatty alcohol citrate, |
| | fatty acid pentaerythritol ester, sorbitan |
| | sesquioleate, white petrolatum, beeswax, |
| | aluminum stearate, butylated |
| | hydroxyanisole (BHA), citric acid, and |
| | optionally water |
| ointment | an alcohol (e.g., ethanol or/and propylene |
| | glycol), polyethylene or white |
| | petrolatum, mineral oil, and optionally water |
| gel | ethanol, carbomer 934P, triethanolamine and water |
| gel | glycerol, carbomer 940, poloxamer, |
| | dimethicone, disodium lauryl sulfosuccinate, |
| | silicon dioxide, a preservative (e.g., |
| | benzoyl peroxide or/and methyl paraben), |
| | EDTA or disodium edetate, a pH adjuster |
| | (e.g., NaOH or lactic acid), and water |
| gel | glycerol, hydroxy-beta-cyclodextrin, |
| | hydroxyethyl cellulose, parabens, EDTA |
| | or disodium edetate, and water |
| gel | propylene glycol, polyacrylic acid, |
| | medium-chain triglycerides, lecithin, |
| | polysorbate 80, a preservative (e.g., |
| | benzoic acid), EDTA or disodium edetate, a |
| | pH adjuster (e.g., NaOH or lactic acid), and water |
| gel | ethanol, isopropyl myristate, carbomer |
| | 940, triethanolamine, docusate sodium, |
| | EDTA or disodium edetate, and water |
| gel | propylene glycol, Carbopol ® 941, PEG |
| | 400, methyl paraben, a pH adjuster (e.g., |
| | NaOH or lactic acid), and water |
| gel | propylene glycol, PEG 400, carbomer 934P, |
| | allantoin, methyl paraben, a pH |
| | adjuster (e.g., NaOH or lactic acid), and water |
| gel | an alcohol (e.g., ethanol or/and propylene |
| | glycol), carbomer, dioctyl sodium |
| | sulfosuccinate, a preservative (e.g., |
| | benzoyl peroxide), a pH adjuster (e.g., NaOH |
| | or lactic acid), and water |
| gel | glycerol, propylene glycol, aloe vera |
| | gel, diazolidinyl urea, |
| | capryl/capramidopropyl betaine, parabens, |
| | citric acid, sodium citrate, and water |
| gel | ethanol, hydroxypropyl cellulose and water |
| lotion | glycerol, stearyl alcohol, glyceryl |
| | stearate, PEG-100 stearate, PEG 400, carbomer |
| | 941, cyclomethicone, light mineral oil, |
| | steareth-21, benzyl alcohol, sorbic acid or |
| | potassium sorbate, a pH adjuster (e.g., |
| | NaOH or lactic acid), and water |
| lotion | isopropanol, propylene glycol, hydroxypropyl |
| | cellulose, sodium phosphate |
| | monobasic, phosphoric acid and water |
| lotion | propylene glycol, cetyl alcohol, stearyl |
| | alcohol, glyceryl stearate, sorbitan |
| | monostearate, light mineral oil, sodium |
| | lauryl sulfate, parabens, EDTA or |
| | disodium edetate, water, and optionally |
| | a pH adjuster (e.g., NaOH or citric acid) |
| lotion | glycerol, cetostearyl alcohol, isostearyl |
| | alcohol, stearic acid, glyceryl stearate, |
| | sodium lauroyl sarcosinate, methyl paraben |
| | and water |
| sup- | an alcohol (e.g., ethanol or/and propylene |
| pository | glycol) and glycerides of saturated fatty |
| | acids |
| sup- | 95% ethanol and Suppocire ® AM (glyceride |
| pository | base containing saturated C₈-C₁₈ |
| | triglyceride fatty acids) |
| pledget | isopropanol, propylene glycol and water |
| foam | ethanol, propylene glycol, cetyl alcohol, |
| | stearyl alcohol, polysorbate 60, KOH and |
| | water, and pressurized with a propane/butane propellant |
| spray | ethanol, undecylenic acid, isopropyl |
| (dermal) | myristate, sodium lauryl sulfate, and water |
| spray | glycerol, lactose, cetostearyl alcohol, |
| (dermal) | mineral oil, ceteth-20 phosphate, dicetyl |
| | phosphate, urea, potassium phosphate monobasic, |
| | parabens, a pH adjuster (e.g., NaOH or |
| | lactic acid), and water |

### Example 6. In Vivo Assay for Cough Sensitization

Hartley guinea pigs are studied in a standard tussive protocol. Briefly, guinea pigs (e.g., n=4-8 per group) are treated with inhalation of nebulized citric acid following prior sensitization with inhaled histamine, and are monitored for the development of coughing nots, as observed by an experienced investigator. Animals receive vehicle (e.g., orally or by nebulized inhalation) or an NK-1 antagonist (e.g., serlopitant, MK-0303 or MK-8478) (e.g., from 0.01 to 3 mg/kg orally or at increasing nebulized concentrations), 30-60 minutes prior to the inhalational challenge of histamine followed by citric acid solution.

### Example 7. Clinical Study of Serlopitant, MK-0303 or MK-8478 for Idiopathic Chronic Cough

A well-controlled human clinical trial testing the efficacy of serlopitant, MK-0303 or MK-8478 in the treatment of idiopathic chronic cough (ICC) is conducted in accordance with the ICH Guidelines for Good Clinical Practices, the U.S. Code of Federal Regulations, the Health Insurance Portability and Accountability Act (HIPAA), and any local regulatory requirements in the U.S. and any other country. The study is a Phase II randomized, double-blind, placebo-controlled, multicenter trial designed to test the efficacy, tolerability and safety of serlopitant, MK-0303 or MK-8478 versus placebo in subjects with ICC. The study patient population includes adult males and females 18-65 years of age. The subjects have ICC of unknown cause and more than 8-week duration despite treatment with standard-of-care antitussive therapies such as oral H₁ antihistamines and corticosteroids.

Subjects are randomized to receive either a 5-mg tablet of serlopitant, MK-0303 or MK-8478 or a matching placebo tablet. Subjects take a tablet of serlopitant, MK-0303 or MK-8478 or placebo once daily by mouth for a total of 8 weeks. The maximum study duration for each subject is approximately 14 weeks and includes a screening period of 2 weeks, a treatment period of 8 weeks, and a follow-up period of 4 weeks. Cough is assessed using an ambulatory sound monitoring system (VitaloJAK™) at baseline and during the 8 weeks of treatment. The study parameters are summarized in Table 6.

TABLE 6

| | A Randomized, Double-Blind, Placebo-Controlled |
|---|---|
| | Study of Serlopitant, MK-0303 or MK-8478 in |
| Study Title: | Subjects with Idiopathic Chronic Cough |
| Development | Phase II |
| Phase: | |
| Study | Primary objective: |
| Objectives: | assess the efficacy of serlopitant, |
| | MK-0303 or MK-8478 in treating |
| | idiopathic chronic cough (ICC) in adults |
| | Secondary objective: |
| | assess the safety and tolerability of repeated |
| | oral doses of serlopitant, MK-0303 or |
| | MK-8478 in the subjects |
| Study Design: | Randomized, double-blind, placebo-controlled study |
| Sample Size: | At least about 100 subjects are randomized. |
| Study | Male and female adults 18-65 years old who have |
| Population: | ICC of unknown cause and more than 8-week duration |
| | despite treatment with standard-of-care antitussive |
| | therapies such as oral H₁ antihistamines |
| | and corticosteroids |
| Investigational | Oral daily tablet of serlopitant, MK-0303 or MK-8478 |
| Product: | |
| Dosage and | Loading dose of three 5-mg tablets of serlopitant, |
| Frequency | MK-0303 or MK-8478 or placebo on Day 1, followed |
| | by one 5-mg tablet of serlopitant, MK-0303 or |
| | MK-8478 or placebo once daily orally in the morning |
| | or at bedtime for 8 weeks |
| Reference | None |
| Product: | |
| Control | Matching placebo once daily for 8 weeks |
| Product: | |
| Efficacy | Primary efficacy endpoint: |
| Evaluation | percent change in daytime or |
| Criteria: | awake objective cough frequency (coughs/hr) |
| | (VitaloJAK ™) from Baseline to Week 8 |
| | Secondary efficacy endpoints: |
| | cough severity and urge to cough - change in |
| | visual analog scale (VAS) score from Baseline |
| | to Week 8 |
| | cough impact - change in Leicester |
| | cough questionnaire (LCQ) and cough quality of |
| | life questionnaire (CQLQ), and global ratings |
| | of change, from Baseline to Week 8 |
| Safety | Safety is assessed by treatment-emergent adverse |
| Evaluation | events, serious adverse events, electrocardiograms, |
| Criteria: | vital signs, and blood and urine laboratory tests. |
| Statistical | The difference in the primary efficacy endpoint |
| Methods: | between treatment groups is tested using a t-test. |
| | The secondary efficacy endpoints are summarized with |
| | descriptive statistics by treatment group, and treatment |
| | differences and 95% confidence intervals are produced. |
| Study Sites: | Multicenter |

Additional or other clinical trials according to a similar study design can be conducted to study, e.g., different dosage levels (e.g., 1 mg or 10 mg daily) or different modes of administration (e.g., oral or nasal inhalation) of serlopitant, MK-0303 or MK-8478, to differentiate between optimal doses or dosing schedules, or to study a different NK-1 antagonist. In addition, the efficacy of the drug in specific patient populations, such as children, adolescents and the elderly, and in treating acute, subacute or chronic cough having an unknown cause or a known cause (e.g., GERD or a respiratory disorder such as asthma or COPD), can be determined in additional or other clinical trials conducted in a similar fashion.

### Example 8. Clinical Study of Serlopitant, MK-0303 or MK-8478 for Refractory Chronic Cough

A well-controlled human clinical trial testing the efficacy of serlopitant, MK-0303 or MK-8478 in the treatment of refractory chronic cough (RCC) is conducted in accordance with the ICH Guidelines for Good Clinical Practices, the U.S. Code of Federal Regulations, HIPAA, and any local regulatory requirements in the U.S. and any other country. The study is a Phase II randomized, double-blind, placebo-controlled, multicenter trial designed to test the efficacy, tolerability and safety of serlopitant, MK-0303 or MK-8478 versus placebo in subjects with RCC. The study patient population includes adult males and females 18-80 years of age. The subjects have a history of RCC, which for purposes of this study is defined as having a diagnosis of treatment-resistant chronic cough or unexplained cough for at least one year.

Subjects are randomized to receive either a 1-mg or 5-mg tablet of serlopitant, MK-0303 or MK-8478 or a matching placebo tablet. Subjects take a tablet of serlopitant, MK-0303 or MK-8478 or placebo once daily by mouth for a total of 12 weeks (84 days). The maximum study duration for each subject is approximately 18 weeks and includes a screening period of 2 weeks, a treatment period of 12 weeks, and a follow-up period of 4 weeks. During the screening period, subjects undergo eligibility evaluation and have baseline cough monitoring conducted. Cough can be assessed using an ambulatory sound monitoring system (VitaloJAK™) at baseline and during the 12 weeks of treatment. The study parameters are summarized in Table 9.

TABLE 9

| | A Randomized, Double-Blind, Placebo-Controlled |
|---|---|
| | Study of Serlopitant, MK-0303 or MK-8478 for |
| Study Title: | the Treatment of Refractory Chronic Cough |
| Development | Phase II |
| Phase: | |
| Study | Primary objective: |
| Objectives: | assess the efficacy of serlopitant, MK- |
| | 0303 or MK-8478 in treating refractory |
| | chronic cough (RCC) in adults |
| | Secondary objective: |
| | assess the safety and tolerability of |
| | repeated oral doses of serlopitant, MK-0303 |
| | or MK-8478 in the subjects |
| Study | Randomized, parallel, double-blind, |
| Design: | placebo-controlled study |
| Sample | About 285 subjects are randomly assigned in an |
| Size: | about 1:1:1 ratio (about 95 per group) to take |
| | orally and daily 1 mg or 5 mg of serlopitant, |
| | MK-0303 or MK-8478, or matching placebo. The |
| | randomization is stratified by country and gender. |
| Study | Male and female adults 18-80 years old who have a |
| Population: | history of RCC, and have a score of ≥40 mm on |
| | the cough severity visual analog scale (VAS) at |
| | screening and Baseline (Day 0) |
| | A subject cannot: |
| | have a chest radiograph or CT thorax within the |
| | last 5 years showing any abnormality considered |
| | to be significantly contributing to chronic cough; |
| | have a history of upper or lower respiratory tract |
| | infection or a significant change in pulmonary |
| | status within 4 weeks of Baseline; |
| | have a known active hepatitis infection; |
| | have a known history of HIV infection; |
| | have a history of cystic fibrosis; |
| | have a history of malignancy within 5 years of |
| | Baseline, except for completely treated and non- |
| | metastatic basal or squamous cell carcinoma of the |
| | skin; |
| | be a current smoker or a person who has |
| | given up smoking within the past 12 months; or |
| | have a history of opioid use within 1 week of Baseline. |
| Investigational | Oral daily tablet of serlopitant, |
| Product: | MK-0303 or MK-8478 |
| Dosage and | Loading dose of three 1-mg or 5-mg tablets of |
| Frequency | serlopitant, MK-0303 or MK-8478 or placebo on |
| | Day 1, followed by one 1-mg or 5-mg tablet of |
| | serlopitant, MK-0303 or MK-8478 or placebo once |
| | daily orally in the morning, and no sooner than |
| | 2 hours before or after a meal, for 12 weeks |
| Reference | None |
| Product: | |
| Control | Matching placebo once daily for 12 weeks |
| Product: | |
| Efficacy | Primary efficacy endpoint: |
| Evaluation | change in 24-hr objective cough frequency |
| Criteria: | (e.g., number of coughs per hour) from Baseline |
| | (Day 0) to Week 12 (Day 84) |
| | Key secondary efficacy endpoints: |
| | change in awake objective cough frequency from |
| | Baseline to Week 12 |
| | proportion of subjects with ≥30% reduction in |
| | 24-hr objective cough frequency per hour from |
| | Baseline to Week 12 |
| | proportion of subjects with ≥30% reduction in |
| | awake objective cough frequency per hour from |
| | Baseline to Week 12 |
| | change in daily cough severity diary (CSD) |
| | score from Baseline to Week 12 |
| | change in cough severity VAS score from Baseline |
| | to Week 12 |
| | Other secondary efficacy endpoints: |
| | change in 24-hr objective cough frequency from |
| | Baseline to Week 4 (Day 28) and Week 8 (Day 56) |
| | change in awake objective cough frequency from |
| | Baseline to Weeks 4 and 8 |
| | change in 24-hr objective cough frequency from |
| | Baseline to Day 112 in the follow-up period |
| | change in sleep objective cough frequency from |
| | Baseline to Weeks 4, 8 and 12 |
| | change in daily CSD score from Baseline to |
| | Weeks 4 and 8 |
| | change in cough severity VAS score from Baseline |
| | to Weeks 4 and 8 |
| | change in Leicester cough questionnaire (LCQ) |
| | individual domain and total scores from Baseline |
| | to Weeks 4, 8 and 12 |
| | change in cough-specific quality of life |
| | questionnaire (CQLQ) individual domain and total |
| | scores from Baseline to Weeks 4, 8 and 12 |
| | patient's global impression of change (PGIC) |
| | clinician's global impression of change (CGIC) |
| Safety | Safety is assessed by adverse events, serious |
| Evaluation | adverse events, electrocardiograms, vital signs, |
| Criteria: | abbreviated physical examinations, and blood |
| | and urine laboratory tests. |
| Statistical | The primary analysis of the primary efficacy |
| Methods: | endpoint is analyzed on the natural log scale. |
| | The difference between each of the two treatment |
| | groups (1 mg and 5 mg of drug) and placebo is |
| | estimated using a mixed model repeated |
| | measures (MMRM) model. The model includes factors |
| | for treatment, visit, country, sex, the interaction |
| | of treatment by visit, and the log-transformed |
| | baseline value as a covariate. The MMRM model uses |
| | all available 24-hr cough frequency data on Days |
| | 28, 56 and 84. The geometric mean of the 24-hr |
| | coughs per hour is presented by treatment and by |
| | visit. The percent difference change between test |
| | compound and placebo is estimated by 100*(e^{diff} − 1), |
| | where diff is the difference provided by the |
| | analysis of the log-transformed variable. To assess |
| | the robustness of efficacy results and the effect of |
| | missing data, additional sensitivity analysis for the |
| | primary efficacy variables is done. |
| Study Sites: | Multicenter |

Additional or other clinical trials according to a similar study design can be conducted to study, e.g., different dosage levels (e.g., 10 mg daily) or different modes of administration (e.g., oral or nasal inhalation) of serlopitant, MK-0303 or MK-8478, to differentiate between optimal doses or dosing schedules, or to study a different NK-1 antagonist. In addition, the efficacy of the drug in specific patient populations, such as children and adolescents, and in treating RCC associated with a particular medical condition (e.g., an interstitial lung disease such as idiopathic pulmonary fibrosis), can be determined in additional or other clinical trials conducted in a similar fashion.

It is understood that, while particular embodiments have been illustrated and described, various modifications can be made thereto and are contemplated herein. It is also understood that the disclosure is not limited by the specific examples provided herein. The description and illustration of embodiments and examples of the disclosure herein are not intended to be construed in a limiting sense. It is further understood that all aspects of the disclosure are not limited to the specific depictions, configurations or relative proportions set forth herein, which may depend upon a variety of conditions and variables. Various modifications and variations in form and detail of the embodiments and examples of the disclosure will be apparent to a person skilled in the art. It is therefore contemplated that the disclosure also covers any and all such modifications, variations and equivalents.

1-46. (canceled)

47. A method of treating cough or urge to cough, comprising orally administering serlopitant or a pharmaceutically acceptable salt, solvate, hydrate, clathrate, polymorph, prodrug, metabolite or stereoisomer thereof, to a subject in need of such treatment according to a schedule, said schedule comprising:
a) first administering at least one loading dose; and
b) second administering at least one therapeutically effective maintenance dose;
wherein said maintenance dose comprises a dosage from about 1 to about 10 mg.

48. The method according to claim 47, wherein the loading dose is 1.5, 2, 3, 4 or 5 times the maintenance dose.

49. The method according to claim 48, wherein the loading dose is 3 times the maintenance dose.

50. The method according to claim 47, wherein the loading dose is administered on day 1 and the maintenance dose is administered on day 2 and thereafter.

51. The method according to claim 47, wherein the maintenance dose is administered in the morning.

52. The method according to claim 47, wherein the maintenance dose is administered without food at least two hours before or after a meal.

53. The method according to claim 47, wherein the maintenance dose is about 5 mg.

54. The method according to claim 47, wherein the maintenance dose is administered once a day, once every two days, once every three days, twice a week, or once a week.

55. The method according to claim 54, wherein the maintenance dose is administered once a day.

56. The method according to claim 55, wherein the maintenance dose comprises a dosage of about 5 mg administered once a day.

57. The method according to claim 47, wherein the loading dose is about 15 mg.

58. The method according to claim 47, wherein the loading dose is about 15 mg, and the maintenance dose is about 5 mg.

59. The method according to claim 47, wherein the serlopitant is administered as a tablet.

60. The method of claim 47, wherein the at least one therapeutically effective maintenance dose of serlopitant is administered over a period of at least 2 weeks, 4 weeks, 6 weeks, 2 months, 3 months, 6 months, 1 year, 2 years or 3 years.

61. The method of claim 60, wherein the at least one therapeutically effective maintenance dose of serlopitant is administered over a period of at least 3 months.

62. The method of claim 47, wherein the cough or urge to cough is chronic cough.

63. The method of claim 62, wherein the chronic cough is idiopathic chronic cough or refractory chronic cough.

64. A method of treating chronic cough, comprising orally administering serlopitant or a pharmaceutically acceptable salt, solvate, hydrate, clathrate, polymorph, prodrug, metabolite or stereoisomer thereof, to a subject in need of such treatment according to a schedule, said schedule comprising:
a) first administering one loading dose of 15 mg serlopitant on day 1; and
b) second administering at least one therapeutically effective maintenance dose of 5 mg serlopitant once a day on day 2 and thereafter;
wherein serlopitant is administered as a tablet and wherein said maintenance dose is administered for at least 3 months.

65. The method of claim 64, wherein the chronic cough is idiopathic chronic cough or refractory chronic cough.
